(19) United States
(12) Patent Application Publication
(10) Pub. No.: US 2005/0089970 A1
(43) Pub. Date: Apr. 28, 2005

(54) Polymer-modified bioactive synthetic chemokines, and methods for their manufacture and use

(76) Inventors: James A Bradburne, Redwood City, CA (US); Gerd G Kochendoerfer, Oakland, CA (US); Jill G Wilken, Walnut Creek, CA (US)

Correspondence Address: LINIAK, BERENATO & WHITE, LLC, 6550 ROCK SPRING DRIVE, SUITE 240, BETHESDA, MD, 20817, US

(21) Appl. No.: 10/332,039

(22) PCT Filed: Jul. 12, 2001
(86) PCT No.: PCT/US01/21933

Related U.S. Application Data
(60) Provisional application No. 60/217,683, filed on Jul. 12, 2000.

(51) Int. Cl. A61K 38/19; C07K 14/52; C07H 21/04; C12P 21/02

(52) U.S. Cl. 435/69.5; 435/320.1; 435/325; 530/351; 424/85.1; 536/23.5

(57) ABSTRACT

The invention relates to polymer-modified bioactive synthetic chemokines and to methods for their production and use. The bioactive synthetic chemokines of the invention comprises a polymer modified polypeptide chemokine backbone. The compounds and methods or the invention are useful for the treatment of disorders involving naturally occurring chemokines, such as for the treatment of HIV and AIDS related disorders and for the treatment of asthma, allergic rhinitis, atopic dermatitis, atheroma/atherosclerosis, organ transplant rejection, and rheumatoid arthritis.

### CROSS-REFERENCE TO RELATED APPLICATIONS

This application is a continuation-in-part of U.S. Patent Application Ser. No. 60/217,683 (filed Jul. 12, 2000), herein incorporated by reference.

### TECHNICAL FIELD

The invention relates to polymer-modified bioactive synthetic chemokines, especially chemokine antagonists and agonists, and methods for their production and use.

### BACKGROUND OF THE INVENTION

Chemokines are small proteins involved in leukocyte trafficking and various other biological processes (Murphy et al., Pharmacological Rev. (2000) 51(1): 145-176, Rollins, BJ., Blood (1997) 90(3):909-928 and Wells et al., Inflammation Res. (1999) 48:353-362). Most chemokines localize and enhance inflammation by inducing chemotaxis and cell activation of different types of inflammatory cells typically present at inflammatory sites. Some chemokines have properties apart from chemotaxis, such as inducing the proliferation and activation of killer cells, modulating growth of haematopoietic progenitor cell types, trafficking of haematopoietic progenitor cells in and out of the bone marrow in inflammatory conditions, angiogenesis and tumor growth. (See, e.g., Baggiolini et al., Ann. Rev. Immunology (1997) 15:675-705; Zlotnik et al., Critical Rev. Immunology (1999) 19(1):1-4; Wang et al., J. Immunological Methods (1998) 220(1-2):1-17; and Moser et al., Intl. Rev. Immunology (1998) 16(3-4):323-344).

The amino acid sequence, structure and function of many chemokines are known. Chemokines have molecular masses of about 8-10 kDa and show approximately 20-50 percent sequence homology among each other at the protein level. The proteins also share common tertiary structures. All chemokines possess a number of conserved cysteine residues involved in intramolecular disulfide bond formation, which are utilized to identify and classify chemokines. For instance, chemokines having the first two cysteine residues separated by a single amino acid are called “C-X-C” chemokines (also called “alpha” chemokines). Chemokines having the first two cysteine residues adjacent are called “CC” chemokines (also called “beta” chemokines). The “C” chemokines differ from the other chemokines by the absence of a cysteine residue (also called “gamma” chemokines). The C chemokines show similarity to some members of the CC chemokines but have lost the first and third cysteine residues that are characteristic of the CC and CXC chemokines. Members of the small group of chemokines with the first two cysteine residues separated by three amino acid are called “CXXXC” chemokines (also called “CX₃C” or “delta” chemokines). There are subgroups of chemokines as well. For instance, CC chemokines containing two additional conserved cysteine residues are known, and sometimes the term “C6-beta” chemokine is used for this subgroup. Most chemokines identified to date are members of the CC and CXC chemokine classes.

Chemokines have been implicated in important disease pathways, such as asthma, allergic rhinitis, atopic dermatitis, cancer, viral diseases, atheroma/atheroschleosis, rheumatoid arthritis and organ transplant rejection. However, a general problem with many chemokines and their potential use as therapeutics relates to their inherent property of promoting or aggravating leukocyte inflammatory responses and infection. To this end, numerous modifications of chemokines have been made in an attempt to generate antagonist of the corresponding wild type chemokine. Proudfoot et al. (J. Biol. Chem. (1996) 271(5):2599-2603); Simmons et al. (Science 276:276-279); Gong et al. (J. Biol. Chem. (1996) 271(18):10521-10527; Baggiolini et al. (J. Exp. Med. (1997) 186(8):1189-1191), Polo et al. (Eur. J. Immunol. (2000) 30:3190-3198), Nibbs et al. (J. Immunol. (2000) 164:1488-1497) and Townson et al. (J. Biol. Chem. (2000) 276(50):39254-39261) report on various N-terminal modified chemokines.

A classic and representative example is the situation for RANTES. Under certain conditions, wild type RANTES can enhance inflammation and HIV infection (Gordon et al., J. Virol. (1999) 73:684-694; Czaplewski et al., J. Biol. Chem. (1999) 274:16077-16084). In contrast, substitutions at positions 26 (E26A) and 66 (E66S) of the RANTES polypeptide chain convert the molecule to its non-inflammatory version and improve its ability to compete with its receptors for HIV (Appay et al., J. Biol. Chem. (1999) 274(39):27505-27512; see also, U.S. Pat. No. 6,214,540, which discloses chemokines that inhibit HIV infection and methods based thereon). Moreover, N-terminal modifications of RANTES have been made that result in antagonists that can block HIV-1 infection, including N-terminal truncation [RANTES 9-68], addition of methionine (“Met-RANTES”), aminooxypentane (“AOP-RANTES”), or nonanoyl (“NNY-RANTES”) (Arenzana-Seisdedos, et al., Nature (1996) 383:400; Mack, et al., J. Exp. Med. (1998) 187:1215-1224; Proudfoot, et al., J. Biol. Chem. (1996) 271:2599-2603; Wells, et al., WO 96/17935; Simmons, et al., Science (1997) 276:276-279; Offord et al., WO 99/11666; and Mosier et al., J Virology (1999) 73(5):3544-3550). U.S. Pat. No. 6,168,784 discloses the chemokine analog NNY-Rantes and suggests modification of the analog with PEG chains at the C-terminus. Wilkin et al. (Curr. Opinion Biotech. (1999) 9:412-426) review chemical synthesis of proteins.

The biological activities of chemokines are mediated by receptors (Murphy et al., Pharmacological Rev. (2000) 51(1):145-176, Rollins, BJ., Blood (1997) 90(3):909-928 and Wells et al., Inflammation Res. (1999) 48:353-362). This includes chemokine-specific receptors as well as receptors with overlapping ligand specificity that bind several different chemokines belonging to either the CC chemokines or the group of CXC chemokines. For instance, the CC chemokine SDF-1α is specific for the CXCR4 receptor, whereas the CXC chemokine RANTES binds to the CCR1, CCR3 and CCR5 receptors. Another example is the chemokine Eotaxin, which is a ligand for the CCR3 (also known as CKR3) receptors. (See, e.g., Cyster, J. G., Science (1999) 286:2098-2102; Ponath et al., J. Experimental Medicine (1996) 183(6):2437-2448; Ponath et al., J. Clinical Investigation (1996) 97(3):604-12; and Yamada et al., Biochem. Biophys. Res. Communications (1997) 231(2): 365-368.

The ability of chemokines to activate their cognate receptors is greatly affected by modifications to the N-terminal region of the chemokines. These changes can result from proteolytic processing, mutagenesis, or chemical modification. (See, e.g., Proudfoot, A. E. et al., J. Biol. Chem. (1996) 271:2599; Grzegorzewski et al., Cytokine (2001) 13(4):209-219; Clark-Lewis et al., J. Biol. Chem. (1991) 266(34):23128-23134; Moser et al., J. Biol. Chem. (1993) 268:7125-7128; Harrison, J. et al., Proc. Nat. Acad. Sci. USA (1998) 95:10896; Mack et al., J. Exp. Med. (1998) 187:1215; Gong et al., J. Biol. Chem. (1996) 271:10521; Struyf et al., Eur. J. Immunol. (1998) 28:1262; Weber et al., J. Exp. Med. (1996) 183:681; Proot et al. (1998) J. Immunol., 160:4034; Yang et al., J. Virol. (1999) 73(6):4582-4589; Rusconi et al., Antivir Ther. (2000) 5(3):199-204; Wyuts et al., J. Immunol. (1999) 163(11):6155-6613; Detheux et al., J. Exp. Med (2000) 192: 1501-1508; Nibbs et al., J Immunology (2000) 164:1488-1497; McCole et al., J Immunol. (1999) 163(5):2829-2835; Nufer et al., Biochemistry (1999) 38(2):636-642; and Wyuts et al., Eur J. Biochem. (1999) 260(2):421-429).

Many of these modified proteins are able to antagonize chemokine receptor-mediated effects in vitro, inhibit viral infection and significantly reduce inflammation in several animal models. In certain cases they retain the ability to activate their receptors, and in primary cells this activity reflects the level of receptor expression. Certain modifications to the N-terminal region also have profound effects on the trafficking of chemokine receptors. Thus while such modified chemokines can antagonize their receptors and corresponding wild type chemokine, classification as antagonist, agonists or variations thereof can differ.

Although chemokines have been proposed as therapeutics, one of the potential major drawbacks is their poor circulating half-life in vivo, typically just a few minutes. To improve circulating half-life, it is known that water-soluble polymers such as PEG (polyethylene glycol) can be attached to proteins, but with mixed results given the difficulty of attaching them in a controlled manner and with user-defined precision (Zalipsky, S., Bioconjugate Chemistry (1995) 6:150-165; Mehvar, R., J. Pharm. Pharm. Pharmaceut. Sci. (2000) 3(1): 125-136; and Monfardini et al., Bioconjugate Chem. (1998) 9:418-450).

In this respect, a technique for the site-specific attachment of PEG chains to the N-terminal residue of proteins was developed and demonstrated on the growth factor granulocyte colony stimulating factor (G-CSF) and the chemokine IL-8 (Gaertner et al., Bioconjug. Chem. (1996) 7(1):38-44). Both were reported to retain most of their activity. However, potency is particularly important for drugs employed as antagonists, such as those used as receptor-based inhibitors of viral infection. Thus, attachment of PEG or other water-soluble polymers to the N-terminal residue of chemokines other than IL-8, particularly chemokine antagonists, may not be suitable given the sensitivity of the N-terminal residue and its contribution to activity and potency. Publication WO 00/53223 is representative of much of the large body of chemokine literature in that it reportedly discloses novel chemokines, and reports that antagonists can be made, and that PEG or other water-soluble polymers can be attached, but with no specificity as to where or what type of chains should be attached, nor any activity associated therewith.

Another potential drawback in using wild type chemokines as therapeutic agents is their tendency to aggregate at high concentrations, and promiscuous binding and differential activation of chemokine receptors (Murphy et al., Pharmacological Rev. (2000) 51(1):145-176), Rollins, B J., Blood (1997) 90(3):909-928; and Wells et al., Inflammation Res. (1999) 48:353-362)). Aggregation can be problematic for formulation and in some instances aggravate pathology (Czaplewski et al., J. Biol. Chem. (1999) 274(23):16077-16084; Czaplewski et al., “Engineering, Biology, and Clinical Development of hMIP-1α,” (1999) In: Chemokines in Disease: Biology and Clinical Research, Ed., C. A. Herbert, Humana Press Inc., Totwa, N. J.; Trkola et al., J. Virol. (1999) 73(8):6370-6379; Appay et al., J. Biol. Chem. (1999) 274(39):27505-27512; Hunter et al., Blood (1995) 86(12):4400-4408; Lord et al., Blood (1995) 85(12):3412-3415; Lord et al., Brit. J Cancer (1996) 74:1017-1022). Promiscuous binding is a hallmark of chemokines, and may be less desirable in some therapeutic settings. Nevertheless, chemokines hold significant promise as therapeutics (Murphy et al., Pharmacological Rev. (2000) 51(1):145-176), Rollins, BJ., Blood (1997) 90(3):909-928; and Wells et al., Inflammation Res. (1999) 48:353-362).

While such approaches have improved antagonist-associated potency in some cases, one of the challenges in making chemokine antagonists is increasing potency while improving other drug properties such as pharmacokinetics. Also, finding a general strategy and method for making potent inhibitors of chemokines and the corresponding chemokine inhibitor compounds and their use in the preparation of medicaments for use in prevention and/or treatment of disease is desired.

Accordingly, there is a need for novel chemokines and modified chemokines that have improved therapeutic properties, including improved circulating half-life and desired activity and potency, and particularly for novel chemokines and modified chemokines that can function to inhibit or antagonize the activity of naturally occurring chemokines. There also is a need to provide chemokines and modified chemokines that have improved circulating half-life and altered receptor activity and potency. The present invention addresses this and other needs.

### SUMMARY OF THE INVENTION

The invention relates to polymer-modified bioactive synthetic chemokines, and especially to N- and/or C-terminally modified chemokines, and to methods for their production and use. The N-terminally modified bioactive synthetic chemokines of the present invention comprise a chemokine polypeptide chain modified at its N-terminus with an aliphatic chain and one or more amino acid derivatives. The C-terminally modified bioactive synthetic chemokines of the present invention comprise a chemokine polypeptide chain modified at its C-terminus with an aliphatic chain or polycyclic. The N- and C-terminally modified bioactive synthetic chemokines of the present invention also may include modifications at both the N- and C-termini in combination. Also provided are methods of production and use of the bioactive synthetic chemokines of the present invention. The present invention is significant in that it provides a general approach for making compounds that are potent antagonists of the corresponding naturally occurring wild type chemokine or their receptors.

### BRIEF DESCRIPTION OF THE DRAWINGS

FIGS. 1A-1E depict schematics of processes for preparing the polymer-modified synthetic bioactive proteins of the invention.

FIGS. 2A-2C depict schematics of processes for preparing synthetic bioactive proteins of the invention.

FIGS. 3A-3B depict schematics of processes for multi-segment ligations that involve the chemical ligation of three or more non-overlapping peptide segments, i.e., at least one segment is a middle segment corresponding to the final full-length ligation product.

FIGS. 4A-4C illustrate native chemical ligation and chemical modification of the resulting side-chain thiol.

FIGS. 5A-5B depict solid phase process for generating the branching core (B) and unique chemoselective functional group (U) of the water-soluble polymer U-B-Polymer-J* of the invention.

FIGS. 6A-6D depict a solid phase process for generating preferred substantially non-antigenic water-soluble polyamide Polymer-J* components of the invention for subsequent attachment to the U-B core.

FIG. 7 depicts process for coupling the U-B component to Polymer-J* component to generate the preferred synthetic polymer constructs of the invention of the formula U-B-Polymer-J*.

FIG. 8 depicts an alternative route for precision attachment of a water-soluble polymer to a peptide segment employable for ligation and production of bioactive synthetic proteins of the invention.

FIG. 9 is a schematic showing a general structure of four classes of naturally occurring chemokines and their corresponding N-terminal, N-loop and C-terminal regions as defined by conserved cysteine patterns, where “C” is one letter code for cysteine and “X” represents any amino acid other than cysteine.

FIGS. 10A-10D depict examples of naturally occurring amino acid sequences of various chemokine polypeptide chains, including the corresponding N-terminal, N-loop and C-terminal regions of these chemokines. The standard one letter amino acid code for the 20 genetically encoded amino acids is used.

FIG. 11 depicts a synthetic chemokine designated Rantes G1755-01, which is a polymer-modified analog of Rantes.

FIG. 12 depicts a synthetic chemokine designated Rantes G1755, which is a polymer-modified analog of Rantes.

FIG. 13 depicts a synthetic chemokine designated Rantes G1805, which is a polymer-modified analog of Rantes.

FIG. 14 depicts a synthetic chemokine designated Rantes G1806, which is a polymer-modified analog of Rantes.

FIG. 15 shows a representative SDS-PAGE gel comparing the relative molecular weights of wild type Rantes to synthetic chemokine analog Rantes G1806 under reducing (R) and non-reducing conditions (N). The relative molecular weights are depicted on the left hand side of each gel, which corresponds to a molecular weight standard run on the same gel.

FIG. 16 shows a representative pharmacokinetic profile comparing plasma concentration in picograms per milliliter (pg/ml) of a given Rantes analog versus time in minutes. The compounds illustrated in this figure are AOP-Rantes and Rantes G1755, G1806, and G1805.

### DESCRIPTION OF THE PREFERRED EMBODIMENTS

The invention is directed to bioactive synthetic chemokines, and especially to N- and/or C-terminally modified chemokine molecules. The novel bioactive synthetic chemokines of the present invention preferably inhibit the activity of a naturally occurring chemokine as determined by a suitable chemokine bioassay. Such molecules may act by antagonizing one or more properties of a chemokine receptor to which they bind (e.g., inhibiting viral infection, causing receptor down-modulation, causing receptor internalization) and thereby “antagonize” the normal cycle of receptor recyling back to the cell surface. In the context of other biological responses, such molecules can act as agonists of a receptor, e.g., inducing calcium flux, initiating chemotaxis, etc. Thus, the bioactive synthetic chemokines of the present invention can act as antagonists (including partial antagonism), but also may act as agonists (including partial agonists), or mixtures of both. Preferred are molecules that exhibit at least one antagonistic property, i.e., an ability to antagonize one or more biological properties of a chemokine receptor to which they bind (e.g., block or partially block (1) viral infection, (2) chemotaxis, (3) receptor cycling etc.). Such molecules may act by binding to (or engaging), but not activating, a chemokine's receptor, or may mediate their action by other means.

A. N- and C-Terminal Modified Bioactive Synthetic Chemokines and of the Present Invention

The invention is particularly directed to bioactive synthetic chemokines that inhibit activity of the corresponding naturally occurring chemokine. Preferably, such molecules possess N- and/or C-terminal modifications. The N-terminally modified bioactive synthetic chemokines of the present invention comprise a chemokine polypeptide chain modified at its N-terminus with an aliphatic chain and one or more amino acid derivatives. The N-terminally modified bioactive synthetic chemokines of the present invention have, as read in the N-terminal to C-terminal direction, the following formula: J1-X1-Z1-CHEMOKINE, where: J1 is an aliphatic chain; X1 is a spacer comprising zero or more amino acids of the N-terminal amino acid sequence of the chemokine polypeptide chain; Z1 is an amino acid derivative; CHEMOKNE is the remaining amino acid sequence of the chemokine polypeptide chain; and the dashes (“-”) represent a covalent bond. The compounds are designed to respect the overall length of the N-terminal region of the polypeptide chain. Accordingly, depending upon the length of the hydrophobic aliphatic chain and the position of the amino acid derivative, the N-terminal antagonist may include one or more substitutions, insertions or deletions at the N-terminus relative to the corresponding naturally occurring chemokine polypeptide chain.

The C-terminally modified bioactive synthetic chemokines of the present invention comprise a chemokine polypeptide chain modified at its C-terminus with an aliphatic chain or polycyclic. These compounds have, as read in the N-terminal to C-terminal direction, the following formula: CHEMOKINE-X2-J2, where: X2 is a spacer comprising zero or more amino acids of the C-terminal amino acid sequence of the chemokine polypeptide chain; J2 is an aliphatic chain or polycyclic; CHEMOKINE is the remaining amino acid sequence of the chemokine polypeptide chain; and the dashes (“-”) represent a covalent bond. The C-terminal region of chemokines is amenable to substantive modification, including insertion, deletion or addition of one or more amino acids or other chemical moieties to extend the C-terminal end of the polypeptide chain compared to the corresponding wild type molecule, as well as addition of fluorescent labels and biocompatible polymers, and conjugation to other compounds such as small organic molecules, peptides, proteins and the like.

The N- and C-terminally modified bioactive synthetic chemokines of the present invention may include modifications at both the N- and C-terminal regions, which when referred to specifically are designated as N-/C-terminally modified bioactive synthetic chemokines. These compounds have the formula J1-X1-Z1-CHEMOKINE-X2-J2, where: J1, X1, Z1, CHEMOKINE, X2, J2 and “-” are as described above. These compounds combine the advantages of the N- and C-terminal modifications in a synergistic manner depending on a given end use.

By “chemokine polypeptide chain” is intended a polypeptide chain that is substantially homologous to the polypeptide chain of a naturally occurring wild type chemokine. By “N-terminal amino acid sequence” is intended the amino acid sequence of the chemokine polypeptide chain that is adjacent and N-terminal to the first disulfide-forming cysteine of the naturally occurring chemokine polypeptide chain. By “C-terminal amino acid sequence” is intended the amino acid sequence of the chemokine polypeptide chain that is adjacent and C-terminal to the last disulfide-forming cysteine of the naturally occurring chemokine polypeptide chain. The chemokine polypeptide chain, the N-terminal amino acid sequence, the C-terminal amino acid sequence, and the first and last disulfide-fortning cysteines forming the basis of a bioactive synthetic chemokine of the present invention can be readily deduced from the corresponding amino acid sequence of the naturally occurring chemokine, as well as by homology modeling with other chemokines of the same class, such as comparison to the amino acid sequences of the known C, CC, CXC and CXXXC chemokines.

For instance, the following are examples of known naturally occurring chemokines, many of which have been described under different names and thus appear several times: 6Ckine, 9E3, ATAC, ABCD-1, ACT-2, ALP, AMAC-1, AMCF-1, AMCF-2, AIF, ANAP, Angie, beta-R1, Beta-Thromboglobulin, BCA-1, BLC, blr-1 ligand, BRAK, C10, CCF18, Ck-beta-6, Ck-beta-8, Ck-beta-8-1, Ck-beta-10, Ck-beta-11, cCAF, CEF4, CINC, C7, CKA-3, CRG-2, CRG-10, CTAP-3, DC-CK1, ELC, Eotaxin, Eotaxin-2, Exodus-1, Exodus-2, ECIP-1, ENA-78, EDNAP, ENAP, FIC, FDNCF, FINAP, Fractalkine, G26, GDCF, GOS-19-1, GOS-19-2, GOS-19-3, GCF, GCP-2, GCP-2-like, GRO1, GRO2, GRO3, GRO-alpha, GRO-beta, GRO-gamma, H400, HC-11, HC-14, HC-21, HCC-1, HCC-2, HCC-3, HCC-4H174, Heparin neutralizing protein, Humig, 1-309, ILINCK, I-TAC, Ifi10, IL8, IP-9, IP-10, IRH, JE, KC, Lymphotactin, L2G25B, LAG-1, LARC, LCC-1, LD78-alpha, LD78-beta, LD78-gamma, LDCF, LEC, Lkn-1, LMC, LAI, LCF, LA-PF4, LDGF, LDNAP, LIF, LIX, LUCT, Lungkine, LYNAP, Manchester inhibitor, MARC, MCAF, MCP-1, MCP-2, MCP-3, MCP4, MCP-5, MDC, MIP-1-alpha, MIP-1-beta, MIP-1-delta, MIP-1-gamma, MIP-3, MIP-3-alpha, MIP-3-beta, MIP-4, MIP-5, Monotactin-1, MPIF-1, MPIF-2, MRP-1, MRP-2, M119, MDNAP, MDNCF, Megakaryocyte-stimulatory-factor, MGSA, Mig, MIP-2, mob-1, MOC, MONAP, NC28, NCC-1, NCC-2, NCC-3, NCC4 N51, NAF, NAP-1, NAP-2, NAP-3, NAP4, NAP S, NCF, NCP, Neurotactin, Oncostatin A, P16, P500, PARC, pAT464, pAT744, PBP, PBP-like, PBSF, PF4, PF4-like, PF4-ALT, PF4V1, PLF, PPBP, RANTES, SCM-1-alpha, SCI, SCY A26, SLC, SMC-CF, ST38, STCP-1, SDF-1-alpha, SDF-1-beta, TARC, TCA-3, TCA-4, TDCF, TECK, TSG-8, TY5, TCF, TLSF-alpha, TLSF-beta, TPAR-1, TSG-1.

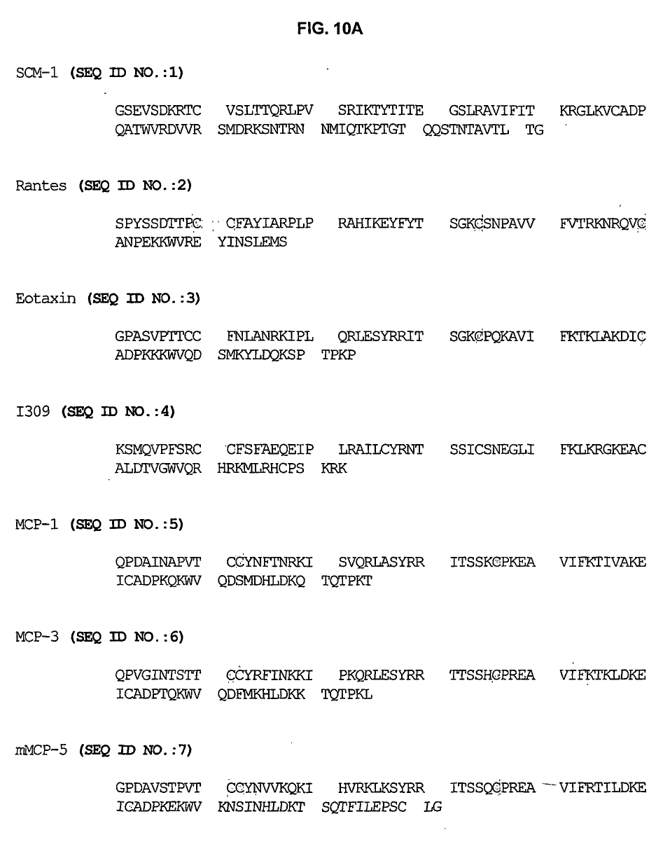

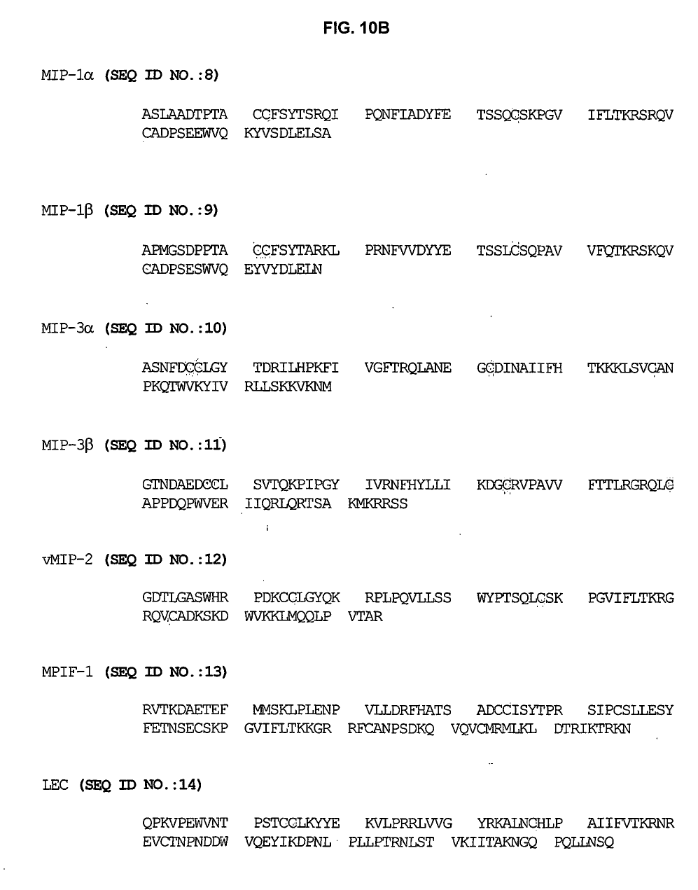

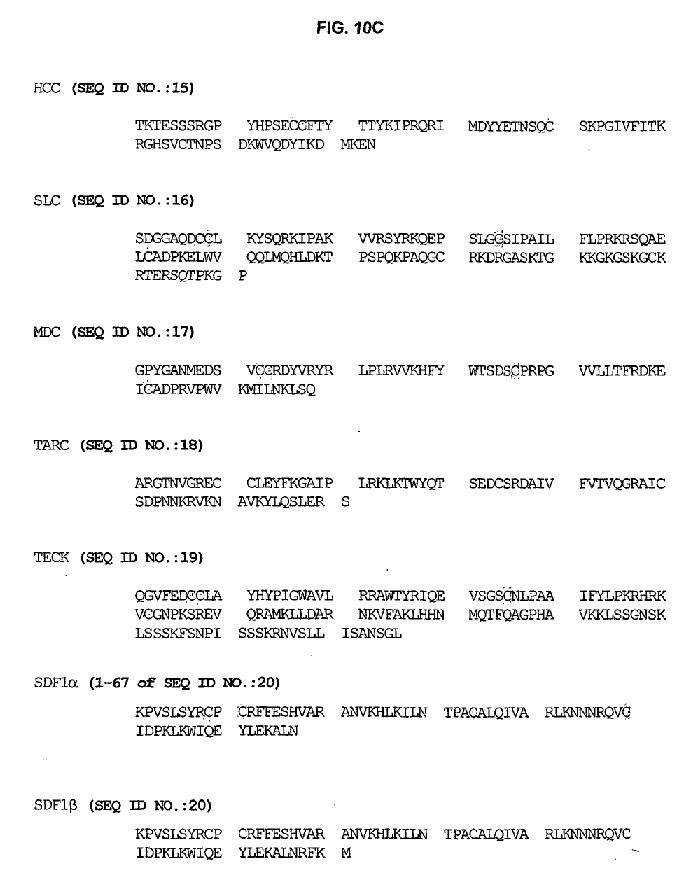

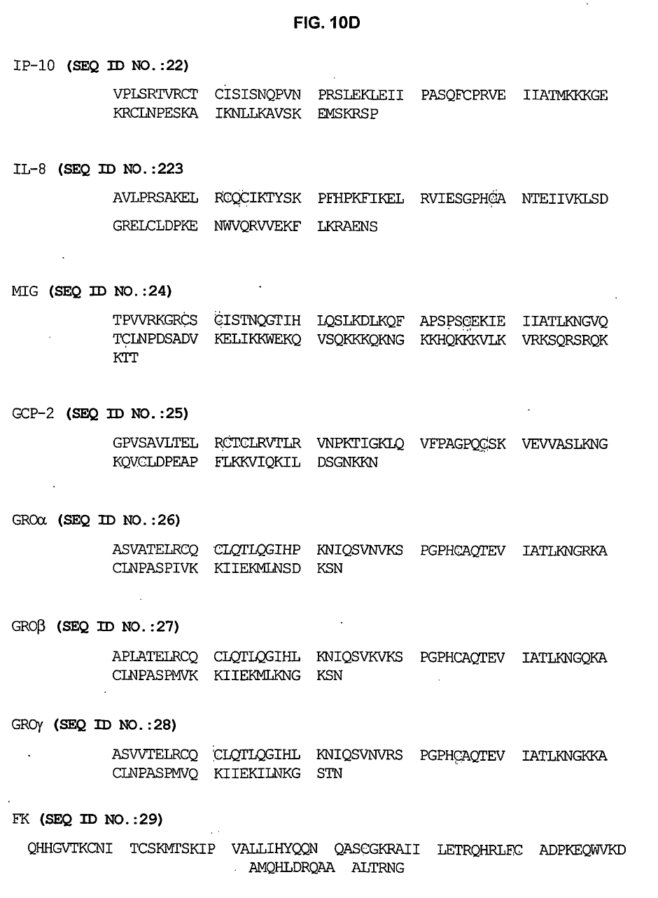

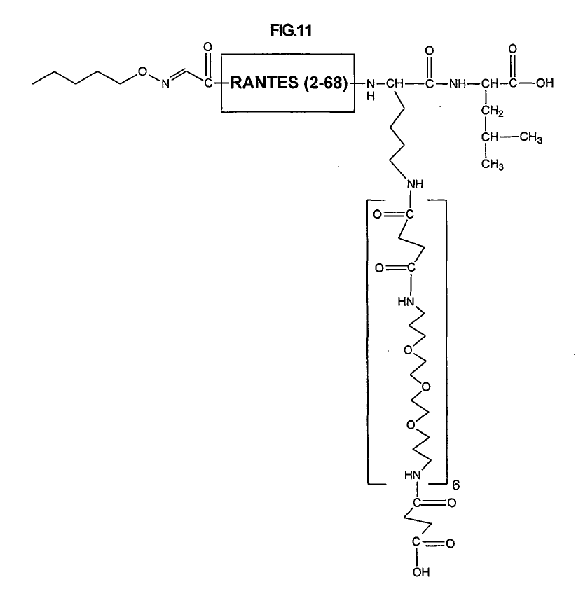

By way of illustration, and not by way of limitation, examples of some of the above-listed wild type chemokine polypeptide chains and their corresponding N-terminal, N-loop and C-terminal amino acid sequences are depicted in FIGS. 10A-10D. As can be appreciated, additional chemokine polypeptide chains are known and obtainable from many different sources including publicly accessible databases such as the Genome Database (Johns Hopkins University, Maryland USA), Protein Data Bank (Brookhaven National Laboratory & Rutgers University, New Jersey USA), Entrez (National Institutes of Health, Maryland USA), NRL 3D (Pittsburgh Supercomputing Center, Carnegie Mellon University, Pennsylvania USA), CATH (University College London, London, UK), NIH Gopher Server (NIH, Maryland USA), ProLink (Boston University, Massachusetts USA), The Nucleic Acid Database (Rutgers University, New Jersey USA), Genebank (National Library of Medicine, Maryland USA), Expasy (Swiss Institute of Bioinformatics, Geneva Switzerland), and the like. Also, new chemokines, such as those derived from various gene and protein sequencing programs can be identified by homology and pattern matching following standard techniques known in the art, including databases and associated tools for achieving this purpose.

In one embodiment of the present invention, directed evolution techniques, such as phage display or modular shuffling, may be used to generate chemokines with increased receptor specificity. The testing of chemokine derivatives or analogues for their ability to bind chemokine receptors using phage display has been described in the treatment and prevention of HIV (U.S. Pat. No. 6,214,540; DeVico et al.). Phage display techniques have also been used to detect or identify ligands, inhibitors or promoters of receptor proteins for CXC Chemokine Receptor 3 (CXCR3) (U.S. Pat. No. 6,140,064, Loetscher et al.), which are characterized by selective binding of one or more chemokines with the ability to induce a cellular response (U.S. Pat. No. 6,184,358, Loetscher et al.). The use of phage display has been described in the labeling and selection of molecules (U.S. Pat. No. 6,180,336, Osbourn et al.), the labeling and subsequent purification of binding molecules for specific antigens (see e.g., WO92/01047), and in the determination of peptide composition for prevention and treatment of HIV infection and immune disorders (U.S. Pat. No. 6,090,388, Wang).

Phage display procedures involving G protein-coupled receptors have also been described (see e.g., Doorbar, J. et al., “Isolation of a peptide antagonist to the thrombin receptor using phage display,” J. Mol. Biol., 244: 361-9 (1994)), with preferred regions for directed evolution at the N-loop region (Konigs, C, “2 Monoclonal antibody screening of a phage-displayed random peptide library reveals mimotopes of chemokine receptor CCR5: implications for the tertiary structure of the receptor and for an N-terminal binding site for HIV-1 gp120,” Eur. J. Immunol. 2000 April; 30(4): 1162-71; Sidhu, S. S. et al., “High copy display of large proteins on phage for functional selections,” J Mol Biol 2000 Feb. 18;296(2):487-95; Fielding, A. K. et al., “A hyperfusogenic gibbon ape leukemia envelope glycoprotein: targeting of a cytotoxic gene by ligand display,” Hum Gene Ther 2000 Apr. 10;11(6):817-26), the region between N-loop and C-terminus, and the C-terminus (Cain, S. A. et al. “Selection of novel ligands from a whole-molecule randomly mutated C5a library,” Protein Eng 2001 March;14(3):189-93; Heller, T. et al., “Selection of a C5a receptor antagonist from phage libraries attenuating the inflammatory response in immune complex disease and ischemia/reperfusion injury,” J. Immunol. 1999 Jul. 15;163(2):985-94; Chang, C. et al., “Dissection of the LXXLL nuclearreceptor-coactivator interaction motif using combinatorial peptide libraries: discovery of peptide antagonists of estrogen receptors alpha and beta,” Mol Cell Biol 1999 December;19(12):8226-39).

Suitable hydrophobic aliphatic chains of J1 and J2 include, but are not limited to, hydrophobic aliphatic chains that are five (C5) to twenty-two (C22) carbons in length. The chain may be unsaturated and/or unbranched, or may have variable degrees of saturation and/or branching. The hydrophobic aliphatic chains have the general formula Cn(Rm)-, where Cn is the number of carbons and Rm is the number of substituent groups selected from hydrogen, alkyl, acyl, aromatic or combination(s) thereof, and n and m may be the same or different. The J1 and J2 groups are joined to X1, X2 or to the chemokine polypeptide chain via any suitable covalent linkage. Examples of suitable covalent linkages include, but are not limited to: amide, ketone, aldehyde, ester, ether, thioether, thioester, thiozolidine, oxime, oxizolidine, Schiff-base and Schiff-base type linkages (for example, hydrazide). Without limitation, such linkages can comprise:
—C(O)—NH—(CH₂)—C(O)—; —C(O)—NH—(CH₂)ₓ—C(O)—; —C(O)—NH—(CH₂)—NH—C(O)—; —C(O)—NH—(CH₂)ₓ—NH—C(O)—; —C(O)—NH—(CH₂)—[(CH₂)—NH]_{y}—C(O)—; —C(O)—NH—(CH₂)—[(CH₂)ₓ—NH]_{y}—C(O)—; —C(O)—NH—(CH₂)—NH—CH₂—C(O)—; —C(O)—NH—(CH₂)—NH—(CH₂)ₓ—C(O)—; —C(O)—NH—(CH₂)—[NH—(CH₂)ₓ]_{y}—; —C(O)—; —C(O)—NH—(CH₂)—[NH—(CH₂)]_{y}—C(O)—;
—NH—(CH₂)—C(O)—; —NH—(CH₂)ₓ—C(O)—; —NH—(CH₂)—NH—C(O)—; —NH—(CH₂)ₓ—NH—C(O)—; —NH—(CH₂)—[(CH₂)—NH]_{y}—C(O)—; —NH—(CH₂)—[(CH₂)ₓ—NH]_{y}—C(O)—; —NH—(CH₂)—NH—CH₂—C(O)—; —NH—(CH₂)—NH—(CH₂)ₓ—C(O)—; —NH—(CH₂)—[NH—(CH₂)ₓ]_{y}—C(O)—; —NH—(CH₂)—[NH—(CH₂)]_{y}—C(O)—;
—ONH—C(O)—; —ONH—(CH₂)—C(O)—; —ONH—(CH₂)ₓ—C(O)—; —ONH—(CH₂)—NH—C(O)—; —ONH—(CH₂)—(CH₂)—NH—C(O)—; —ONH—(CH₂)ₓ—NH—C(O)—; —ONH—(CH₂)—[(CH₂)—NH]_{y}—C(O)—; —ONH—(CH₂)—[(CH₂)ₙ—NH]_{y}—C(O)—; —ONH—(CH₂)—NH—CH₂—C(O)—; —ONH—(CH₂)—NH—(CH₂)ₓ—C(O)—; —ONH—(CH₂)—[NH—(CH₂)ₓ]_{y}—C(O)—; —ONH—(CH₂)—[NH—(CH₂)]_{y}—C(O)—;
—OCH₂—C(O)—; —OCH₂—(CH₂)—C(O)—; —OCH₂—(CH₂)ₓ—C(O)—; —OCH₂—(CH₂)—NH—C(O)—; —OCH₂—(CH₂)—(CH₂)—NH—C(O)—; —OCH₂—(CH₂)ₓ—NH—C(O)—; —OCH₂—(CH₂)—[(CH₂)—NH]_{y}—C(O)—; —OCH₂—(CH₂)—[(CH₂)ₓ—NH]_{y}—C(O)—; —OCH₂—(CH₂)—NH—CH₂—C(O)—; —OCH₂—(CH₂)—NH—(CH₂)ₓ—C(O)—; —OCH₂—(CH₂)—[NH—(CH₂)ₓ]_{y}—C(O)—; —OCH₂—(CH₂)—[NH—(CH₂)]_{y}—C(O)—; —OCH₂—NH—C(O)—; —OCH₂—NH—(CH₂)—C(O)—; —OCH₂—NH—(CH₂)ₓ—C(O)—; —OCH₂—NH—(CH₂)—NH—C(O)—; —OCH₂—NH—(CH₂)—(CH₂)—NH—C(O)—; —OCH₂—NH—(CH₂)ₓ—NH—C(O)—; —OCH₂—NH—(CH₂)—[(CH₂)—NH]_{y}—C(O)—; —OCH₂—NH—[(CH₂)ₓ—NH]_{y}—C(O)—; —OCH₂—(CH₂)—NH—CH₂—C(O)—; —OCH₂—(CH₂)—NH—(CH₂)ₓ—C(O)—; —OCH₂—(CH₂)—[NH—(CH₂)ₓ]_{y}—C(O)—; —OCH₂—(CH₂)—[NH—(CH₂)]_{y}—C(O)—; —OCH₂—N(CH₃)—C(O)—; —OCH₂—N(CH₃)—(CH₂)—C(O)—; —OCH₂—N(CH₃)—(CH₂)ₓ—C(O)—; —OCH₂—N(CH₃)—(CH₂)—NH—C(O)—; —OCH₂—N(CH₃)—(CH₂)ₓ—NH—C(O)—; —OCH₂—N(CH₃)—(CH₂)ₓ—NH—C(O)—; —OCH₂—N(CH₃)—(CH₂)—[(CH₂)—NH]_{y}—C(O)—; —OCH₂—N(CH₃)—(CH₂)—[(CH₂)ₓ—NH]_{y}—C(O)—; —OCH₂—N(CH₃)—(CH₂)—NH—CH₂—C(O)—; —OCH₂—N(CH₃)—(CH₂)—NH—(CH₂)ₓ—C(O)—; —OCH₂—N(CH₃)—(CH₂)—[NH—(CH₂)ₓ]_{y}—C(O)—; —OCH₂—N(CH₃)—(CH₂)—[NH—(CH₂)]_{y}—C(O)—;
—O—C(O)—C(O)—; —O—C(O)—(CH₂)—C(O)—; —O—C(O)—(CH₂)ₓ—C(O)—; —O—C(O)—(CH₂)—NH—C(O)—; —O—C(O)—(CH₂)—(CH₂)—NH—C(O)—; —O—C(O)—(CH₂)ₓ—NH—C(O)—; —O—C(O)—(CH₂)—[(CH₂)—NH]_{y}—C(O)—; —O—C(O)—(CH₂)—[(CH₂)ₓ—NH]_{y}—C(O)—; —O—C(O)—(CH₂)—NH—CH₂—C(O)—; —O—C(O)—(CH₂)—NH—(CH₂)ₓ—C(O)—; —O—C(O)—(CH₂)—[NH—(CH₂)ₓ]_{y}—C(O)—; —O—C(O)—(CH₂)—[NH—(CH₂)]_{y}—C(O)—; —O—C(O)—NH—C(O)—; —O—C(O)—NH—(CH₂)—C(O)—; —O—C(O)—NH—(CH₂)ₓ—C(O)—; —O—C(O)—NH—(CH₂)—NH—C(O)—; —O—C(O)—NH—(CH₂)—(CH₂)—NH—C(O)—; —O—C(O)—NH—(CH₂)ₙ—NH—C(O)—; —O—C(O)—NH—(CH₂)—[(CH₂)—NH]_{y}—C(O)—; —O—C(O)—NH—[(CH₂)ₓ—NH]_{y}—C(O)—; —O—C(O)—(CH₂)—NH—CH₂—C(O)—; —O—C(O)—(CH₂)—NH—(CH₂)ₓ—C(O)—; —O—C(O)—(CH₂)—[NH—(CH₂)ₓ]_{y}—C(O)—; —O—C(O)—(CH₂)—[NH—(CH₂)]_{y}—C(O); —O—C(O)—N(CH₃)—C(O)—; —O—C(O)—N(CH₃)—(CH₂)—C(O)—; —O—C(O)—N(CH₃)— (CH₂)ₓ—C(O)—; —O—C(O)—N(CH₃)—(CH₂)—NH—C(O)—; —O—C(O)—N(CH₃)—(CH₂)ₓ—NH—C(O)—; —O—C(O)—N(CH₃)—(CH₂)ₓ—NH—C(O)—; —O—C(O)—N(CH₃)— (CH₂)—[(CH₂)—NH]_{y}—C(O)—; —O—C(O)—N(CH₃)— (CH₂)—[(CH₂)ₓ—NH]_{y}—C(O)—; —O—C(O)—N(CH₃)— (CH₂)—NH—CH₂—C(O)—; —O—C(O)—N(CH₃)—(CH₂)—NH—(CH₂)ₓ—C(O)—; —O—C(O)—N(CH₃)—(CH₂)—[NH—(CH₂)ₓ]_{y}—C(O)—; —O—C(O)—N(CH₃)— (CH₂)—[NH—(CH₂)]_{y}—C(O)—;
—CH═CH—C(O)—; —CH═CH—(CH₂)—C(O)—; —CH═CH—(CH₂)ₓ—C(O)—; —CH═CH—(CH₂)—NH—C(O)—; —CH═CH—(CH₂)ₓ—NH—C(O)—; —CH═CH—(CH₂)—[(CH₂)—NH)_{y}—C(O)—; —CH═CH—(CH₂)—[(CH₂)ₓ—NH]_{y}—C(O)—; —CH═CH—(CH₂)—NH—CH₂—C(O)—; —CH═CH—(CH₂)—NH—(CH₂)ₓ—C(O)—; —CH═CH—(CH₂)—[NH—(CH₂)]_{y}—C(O)—; —CH═CH—(CH₂)—[NH—(CH₂)ₓ]_{y}—C(O)—; —SCH₂—N(CH₃)—C(O)—; —SCH₂—N(CH₃)—(CH₂)—C(O)—; —SCH₂—N(CH₃)— (CH₂)ₓ—C(O)—; —SCH₂—N(CH₃)—(CH₂)—NH—C(O)—; —SCH₂—N(CH₃)—(CH₂)ₓ—NH—C(O)—; —SCH₂—N(CH₃)—(CH₂)ₓ—NH—C(O)—; —SCH₂—N(CH₃)—(CH₂)—[(CH₂)—NH]_{y}—C(O)—; —SCH₂—N(CH₃)—(CH₂)—[(CH₂)ₓ—NH]_{y}—C(O)—; —SCH₂—N(CH₃)—(CH₂)—NH—CH₂—C(O)—; —SCH₂—N(CH₃)—(CH₂)—NH—(CH₂)ₓ—C(O)—; —SCH₂—N(CH₃)—(CH₂)—[NH—(CH₂)ₓ]_{y}—C(O)—; —SCH₂—N(CH₃)—(CH₂)—[NH—(CH₂)]_{y}—C(O)—;
—S—C(O)—C(O)—; —S—C(O)—(CH₂)—C(O)—; —S—C(O)—(CH₂)ₓ—C(O)—; —S—C(O)—(CH₂)—NH—C(O)—; —S—C(O)—(CH₂)—(CH₂)—NH—C(O)—; —S—C(O)—(CH₂)ₓ—NH—C(O)—; —S—C(O)—(CH₂)—[(CH₂)—NH]_{y}—C(O)—; —S—C(O)—(CH₂)—[(CH₂)ₓ—NH]_{y}—C(O)—; —S—C(O)—(CH₂)—NH—CH₂—C(O)—; —S—C(O)—(CH₂)—NH—(CH₂)ₓ—C(O)—; —S—C(O)—(CH₂)—[NH—(CH₂)ₓ]_{y}—C(O)—; —S—C(O)—(CH₂)—[NH—(CH₂)]_{y}—C(O)—; —S—C(O)—NH—C(O)—; —S—C(O)—NH—(CH₂)—C(O)—; —S—C(O)—NH—(CH₂)ₓ—C(O)—; —S—C(O)—NH—(CH₂)—NH—C(O)—; —S—C(O)—NH—(CH₂)—(CH₂)—NH—C(O)—; —S—C(O)—NH—(CH₂)ₓ—NH—C(O)—; —S—C(O)—NH—(CH₂)—[(CH₂)—NH]_{y}—C(O)—; —S—C(O)—NH—[(CH₂)ₓ—NH]_{y}—C(O)—; —S—C(O)—(CH₂)—NH—CH₂—C(O)—; —S—C(O)—(CH₂)—NH—(CH₂)ₓ—C(O)—; —S—C(O)—(CH₂)—[NH—(CH₂)ₓ]_{y}—C(O)—; —S—C(O)—(CH₂)—[NH—(CH₂)]_{y}—C(O)—; —S—C(O)—N(CH₃)—C(O)—; —S—C(O)—N(CH₃)—(CH₂)—C(O)—; —S—C(O)—N(CH₃)—(CH₂)ₓ—C(O)—; —S—C(O)—N(CH₃)—(CH₂)—NH—C(O)—; —S—C(O)—N(CH₃)—(CH₂)ₓ—NH—C(O)—; —S—C(O)—N(CH₃)—(CH₂)ₓ—NH—C(O)—; —S—C(O)—N(CH₃)—(CH₂)—[(CH₂)—NH]_{y}—C(O)—; —S—C(O)—N(CH₃)— (CH₂)—[(CH₂)ₓ—NH]_{y}—C(O)—; —S—C(O)—N(CH₃)—(CH₂)—NH—CH₂—C(O)—; —S—C(O)—N(CH₃)—(CH₂)—NH—(CH₂)ₓ—C(O)—; —S—C(O)—N(CH₃)— (CH₂)—[NH—(CH₂)ₓ]_{y}—C(O)—; —S—C(O)—N(CH₃)—(CH₂)—[NH—(CH₂)]_{y}—C(O)—; —C₃H₆SN—C(O)—; —C₃H₆SN—(CH₂)—C(O)—; —C₃H₆SN—(CH₂)ₓ—C(O)—; —C₃H₆SN—(CH₂)—NH—C(O)—; —C₃H₆SN—(CH₂)—(CH₂)—NH—C(O)—; —C₃H₆SN—(CH₂)ₓ—NH—C(O)—; —C₃H₆SN—(CH₂)—[(CH₂)—NH]_{y}—C(O)—; —C₃H₆SN—(CH₂)—[(CH₂)ₓ—NH]_{y}—C(O)—; —C₃H₆SN—(CH₂)—NH—CH₂—C(O)—; —C₃H₅SN—(CH₂)—NH—(CH₂)ₓ—C(O)—; —C₃H₆SN—(CH₂)—[NH—(CH₂)ₓ]_{y}—C(O)—; —C₃H₆SN—(CH₂)—[NH—(CH₂)]_{y}—C(O)—; —C₃H₆SN—NH—C(O)—; —C₃H₆SN—NH—(CH₂)—C(O)—; —C₃H₆SN—NH—(CH₂)ₓ—C(O)—; —C₃H₆SN—NH—(CH₂)—NH—C(O)—; —C₃H₆SN—NH—(CH₂)—(CH₂)—NH—C(O)—; —C₃H₆SN—NH—(CH₂)ₓ—NH—C(O)—; —C₃H₆SN—NH—(CH₂)—[(CH₂)—NH]_{y}—C(O)—; —C₃H₆SN—NH—[(CH₂)ₓ—NH]_{y}—C(O)—; —C₃H₆SN—(CH₂)—NH—CH₂—C(O)—; —S—C(O)—(CH₂)—NH—(CH₂)ₓ—C(O)—; —C₃H₆SN—(CH₂)—[NH—(CH₂)ₓ]_{y}—C(O)—; —C₃H₆SN—(CH₂)—[NH—(CH₂)]_{y}—C(O)—; —C₃H₆SN—N(CH₃)—C(O)—; —C₃H₆SN—N(CH₃)—(CH₂)—C(O)—; —C₃H₆SN—N(CH₃)—(CH₂)ₓ—C(O)—; —C₃H₆SN—N(CH₃)—(CH₂)—NH—C(O)—; —C₃H₆SN—N(CH₃)—(CH₂)ₓ—NH—C(O)—; —C₃H₆SN—N(CH₃)—(CH₂)ₓ—NH—C(O)—; —C₃H₆SN—N(CH₃)—(CH₂)—[(CH₂)—NH]_{y}—C(O)—; —C₃H₆SN—N(CH₃)— (CH₂)—[(CH₂)_{y}—NH]_{y}—C(O)—; —C₃H₆SN—N(CH₃)—(CH₂)—NH—CH₂—C(O)—; —C₃H₆SN—N(CH₃)— (CH₂)—NH—(CH₂)ₓ—C(O)—; —C₃H₆SN—N(CH₃)—(CH₂)—[NH—(CH₂)ₓ]_{y}—C(O)—; —C₃H₆SN—N(CH₃)—(CH₂)—[NM—(CH₂)]_{y}—C(O)—;
—C₃H₆ON—C(O)—; —C₃H₆ON—(CH₂)—C(O)—; —C₃H₆ON—(CH₂)ₙ—C(O)—; —C₃H₆ON—(CH₂)—NH—C(O)—; —C₃H₆ON—(CH₂)—(CH₂)—NH—C(O)—; —C₃H₆ON—(CH₂)ₓ—NH—C(O)—; —C₃H₆ON—(CH₂)—[(CH₂)—NH]_{y}—C(O)—; —C₃H₆ON—(CH₂)—[(CH₂)ₓ—NH]_{y}—C(O)—; —C₃H₆ON—(CH₂)—NH—CH₂—C(O)—; —C₃H₆ON—(CH₂)—NH—(CH₂)ₙ—C(O)—; —C₃H₆ON—(CH₂)—NH—(CH₂)ₓ]_{y}—C(O)—; —C₃H₆ON—(CH₂)—[H—(CH₂)]_{y}—C(O)—; —C₃H₆ON—NH—C(O)—; —C₃H₆ON—NH—(CH₂)—C(O)—; —C₃H₆ON—NH—(CH₂)ₓ—C(O)—; —C₃H₆ON—NH—(CH₂)—NH—C(O)—; —C₃H₆ON—NH—(CH₂)—(CH₂)—NH—C(O)—; —C₃H₆ON—NH—(CH₂)ₓ—NH—C(O)—; —C₃H₆ON—NH—(CH₂)—[(CH₂)—NH]_{y}—C(O)—; —C₃H₆ON—NH—[(CH₂)ₓ—NH]_{y}—C(O)—; —C₃H₆ON—(CH₂)—NH—CH₂—C(O)—; —S—C(O)—(CH₂)—NH—(CH₂)ₙ—C(O)—; —C₃H₆ON—(CH₂)—[NH—(CH₂)ₓ]_{y}—C(O)—; —C₃H₆ON—(CH₂)—[NH—(CH₂)]_{y}—C(O)—; —C₃H₆ON—N(CH₃)—C(O)—; —C₃H₆ON—N(CH₃)—(CH₂)—C(O)—; —C₃H₆ON—N(CH₃)—(CH₂)ₓ—C(O)—; —C₃H₆ON—N(CH₃)—(CH₂)—NH—C(O)—; —C₃H₆ON—N(CH₃)—(CH₂)ₓ—NH—C(O)—; —C₃H₆ON—N(CH₃)—(CH₂)ₙ—NH—C(O)—; —C₃H₆ON—N(CH₃)— (CH₂)—[(CH₂)—NH]_{y}—C(O)—; —C₃H₆ON—N(CH₃)— (CH₂)—[(CH₂)ₓ—NH]_{y}—C(O)—; —C₃H₆ON—N(CH₃)—(CH₂)—NH—CH₂—C(O)—; —C₃H₆ON—N(CH₃)— (CH₂)—NH—(CH₂)ₙ—C(O)—; —C₃H₆ON—N(CH₃)— (CH₂)—[NH—(CH₂)ₓ]_{y}—C(O)—; —C₃H₆ON—N(CH₃)— (CH₂)—[NH—(CH₂)]_{y}—C(O)—; —O—C(O)—; —C(O)—, or a covalent bond, where x and y are 2, 3, 4 or more, and may be the same or different.

Chemistries suitable for linkage systems are well known and can be utilized for this purpose (see, for example, “Chemistry of Protein Conjugation and Cross-Linking”, S. S. Wong, Ed., CRC Press, Inc. (1993); Perspectives in Bioconjugate Chemistry, Claude F. Modres, Ed., ACS (1993)).

In addition to joining J1 and J2 to X1, X2 or the chemokine polypeptide chain, the linkage system employed can be selected to tune the physical-chemical and/or biological properties of the target molecule, provided that the resulting molecule retains its antagonist properties. This can be accomplished, for example, by incorporating a linkage system that is more (or less) stable under one type of condition compared to another for modulating half-life and the like, or for tuning potency, specificity and the like by utilizing linkage systems of variable length, rigidity, charge and/or chirality. The linkage unit joining the hydrocarbon chains to the chemokine polypeptide chain can vary substantially, with the proviso that the overall length and space filling of J1 and/or J2 will most preferably approximate that of the naturally occurring chemokine.

In a preferred embodiment, the hydrophobic aliphatic chain J1 is a hydrocarbon chain five (C5) to ten (C10 carbons in length, and the hydrophobic aliphatic chain J2 is a lipid 12 (C12) to twenty (C20) carbons in length. Examples of the J1 C5-C10 hydrocarbon chains include, but are not limited to: —C₅H₁₁, —C₅H₉, —C₅H₇, —C₅H₅, —C₅H₃, —C₆H₁₃, —C₆H₁₁, —C₆H₉, —C₆H₇, —C₆C₅, —C₆H₃, —C₇H₁₅, —C₇H₁₃, —C₇H₁₁, —C₇H₉, —C₇H₇, —C₇H₅, —C₇H₃, —C₈H₁₇, —C₈H₁₅, —C₈H₁₃, —C₈H₁₁, —C₈H₉, —C₈H₇, —C₈H₅, —C₈H₃, —C₉H₁₉, CH₇, —C₉H₁₅, —C₉H₁₃, —C₉H₁₁, —C₉H₉, —C₉H₇, —C₉H₅, —C₉H₃, —C₁₀H₂₁, —C₁₀H₁₉, C₁₀H₁₇, —C₁₀H₁₅, —C₁₀H₁₃, —C₁₀H₁₁, —C₁₀H₉, —C₁₀H₇, —C₁₀H₅, and —C₁₀H₃.

Suitable J2 lipids include, but are not limited to the fatty acid derived lipids and polycyclic steroid derived lipids. The fatty acids include, but are not limited to, saturated and unsaturated fatty acids. Examples of saturated fatty acids are lauric acid (C12), myristic acid (C 14), palmitic acid (C16), steric acid (C18), and arachidic acid (C20). Examples of unsaturated fatty acids include oleic acid (C18), linoleic acid (C 18), linolenic acid (C 18), eleosteric acid (C18), and arachidonic acid (C20). The polycyclics include, but are not limited to: aldosterone, cholestanol, cholesterol, cholic acid, coprostanol, corticosterone, cortisone, dehydrocholesterol, desmosterol, digitogenin, ergosterol, estradiol, hydoxycorticosterone, lathosterol, prednisone, pregnenolone, progesterone, testosterone, zymosterol, etc. The fatty acids are usually joined to the chemokine polypeptide chain through the acid component, thereby yielding an acyl-linked moiety, although other linkages may be employed. The linkage unit joining the hydrocarbon chains to the chemokine polypeptide chain can vary substantially, with the proviso that the overall length and space filling of the N-terminal region approximates that of the naturally occurring chemokine. The C-terminal region has been found to be more flexible in this regard, so the overall length and space filling can be varied to a greater extent than with the N-terminal region.

In another preferred embodiment, the J1 and J2 components when comprised in a bioactive synthetic chemokine of the invention comprise a C5 to C₂₋₀ saturated or unsaturated acyl chain, such as nonanoyl, nonenoyl, aminooxypentane, dodecanoyl, myristoyl, palmitate, lauryl, palmitoyl, eicosanoyl, oleoyl, or cholyl. For example, the J1 substituent can be nonaoyl or aminooxypentane and the J2 substituent can be a saturated or unsaturated fatty acid, preferably a C12-C20 fatty acid, or a polycyclic steroid lipid such as cholesterol.

Depending upon the nature and length of the hydrophobic aliphatic chain, the bioactive synthetic chemokines of the present invention may include additional amino acids or other moieties that are added to the polypeptide chain, particularly at the C-terminal end to provide a spacer group and/or separate attachment site for the hydrophobic aliphatic moiety.

By “amino acid derivative” is intended an amino acid or amino acid-like chemical entity other than one of the 20 genetically encoded naturally occurring amino acids. In particular, the amino acid derivative Z1 is other than one of the 20 genetically encoded naturally occurring amino acids, and has the formula —(N—CnR—CO)—, where Cn is 1-22 carbons, R is hydrogen, alkyl or aromatic, and where N and Cn, N and R, or Cn and R can form a cyclic structure. Also, N, Cn and R can each have one or more hydrogens in its reduced form depending on the amino acid derivative. The alkyl moiety can be substituted or non-substituted, its can be linear, branched, or cyclic, and may include one or more heteroatoms. The aromatic can be substituted or non-substituted, and include one or more heteroatoms. The amino acid derivatives can be made de novo or obtained from commercial sources (See, e.g., Calbiochem-Novabiochem AG, Switzerland; Advanced Chemtech, Louisville, Ky., USA; Lancaster Synthesis, Inc., Windham, N.H., USA; Bachem California, Inc., Torrance, Calif., USA; Genzyme Corp., Cambridge, Mass., USA). Examples of amino acid derivatives include, but are not liited to, aminoisobutyric acid (Aib), hydroxyproline (Hyp), 1,2,3,4-tetrahydroisoquinoline-3-COOH (Tic), indoline-2-carboxylic acid (indol), 4-difluoro-proline (P(4,4DiF)), L-thiazolidine-4-carboxylic acid (Thz), L-homoproline (HoP), 3,4-dehydro-proline (ΔPro), 3, 4dihydroxyphenylalanine (F(3,4-DiOH)), pBzl,-3, 4dihydroxyphenylalanine (F(3,4-DiOH, pBzl)), benzophenone (p-Bz), cyclohexyl-alanine (Cha), 3-(2-naphtyl)-alanine (βNal), cyclohexyl-glycine (Chg), and phenylglycine (Phg).

With respect to X1, CHEMOKINE and X2, the amino acid sequence of these components is substantially homologous to the corresponding naturally occurring wild type molecule. The term “substantially homologous” when used herein includes amino acid sequences having at least 40%, 50%, 60%, 70%, 80%, 90%, 95% or 99% sequence homology with the given sequence (95-99% preference). This term can include, but is not limited to, amino acid sequences having from 1 to 20, from 1 to 10 or from 1 to 5 single amino acid deletions, insertions or substitutions relative to a given sequence provided that the resultant polypeptide acts as an antagonist of the corresponding naturally occurring chemokine.

For instance, it is well known in the art that certain amino acids can be replaced with others resulting in no substantial change in the properties of a polypeptide, including but not limited to conservative substitutions of amino acids. Such possibilities are within the scope of the present invention. It should also be noted that deletions or insertions of amino acids can often be made which do not substantially change the properties of a polypeptide. The present invention includes such deletions or insertions (which may be, for example up to 10, 20 or 50% of the length of the specific antagonist's sequence of the corresponding naturally occurring chemokine). Moreover, chemokines may be subjected to substantial modifications, including mixing and matching different chemokine polypeptide segments to create additional diversity, such as the modular ‘cross-over’ synthesis approach described in WO 99/11655, which reference is incorporated herein in its entirety by reference.

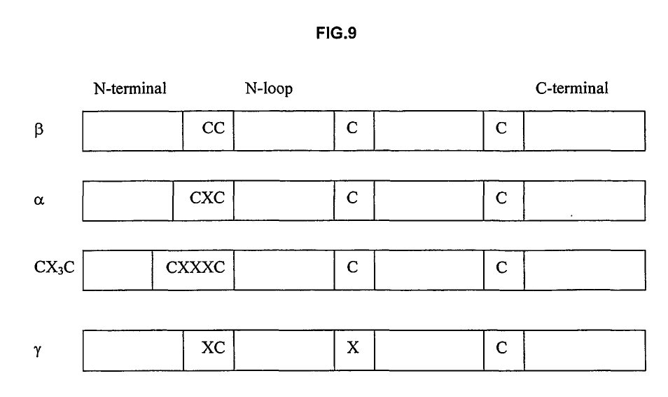

In addition to changes at the N- and/or C-terminus, the bioactive synthetic chemokines of the present invention also may include one or more amino acid substitutions, insertions or deletions elsewhere in the polypeptide chain, i.e., in the polypeptide chain represented in the above formulae by CHEMOKINE. In a preferred embodiment, changes are made in the N-loop of the chemokine to increase its specificity/selectivity for a target receptor. In this way, the N-loop of the bioactive synthetic chemokine of the present invention may block a specific receptor while minimizing the antagonist effect on other of its possible co-receptors. By “4N-loop” is intended the 20 to 26 amino acid sequence region adjacent/C-terminal to the first conserved cysteine pattern defining the N-terminal region of a given chemokine polypeptide chain (see, FIGS. 9 and 10A-10D). For example, as read in the N— to C-terminal direction of the chemokine polypeptide chain, the N-loop of a CC chemokine is the region of amino acids located between and adjacent/C-terminal to the first and second conserved cysteine amino acids and adjacent/N-terminal to the third conserved cysteine amino acid.

In another embodiment, the substitutions and insertions may include natural amino acids as well as amino acid derivatives or amino acids modified with a polymer. The preferred location for polymer attachment is at the C-terminal portion of the chemokine. For chemokines that have natural glycosylation sites, the polymer may be attached to one or more of the amino acids that is coded for glycosylation, e.g., an arginine of an N-linked glycosylation site. The polymer attachment may be to the side chain of the naturally occurring amino acid, an amino acid derivative that replaces the naturally occurring amino acid, or to a carbohydrate or other moiety that is attached to the side chain of the amino acid at the target glycosylation position. Polymers suitable for these purposes are biocompatible, namely, they are non-toxic to biological systems, and many such polymers are known. Such polymers may be hydrophobic or hydrophilic in nature, biodegradable, non-biodegradable, or a combination thereof. These polymers include, but are not limited to, natural polymers such as collagen, gelatin, cellulose, hyaluronic acid, polysaccharides, and polyamino acids, as well as synthetic polymers such as polyesters, polyorthoesters, polyanhydrides, and the like. Examples of hydrophobic non-degradable polymers include polydimethyl siloxanes, polyurethanes, polytetrafluoroethylenes, polyethylenes, polyvinyl chlorides, and polymethyl methacrylates. Examples of hydrophilic non-degradable polymers include poly(2-hydroxyethyl methacrylate), polyvinyl alcohol, poly(N-vinyl pyrrolidone), polyalkylenes, polyacrylamide, and co-polymers thereof. Preferred polymers comprise as a sequential repeat unit ethylene oxide of the formula: —(CH₂—CH₂—O—)ₙ—, where n=the number of ethylene oxide units. Examples of preferred ethylene oxide containing polymers are polyethylene glycol (“PEG”), and polyamide ethylene oxides, such as described in below and WO 00/12587, respectively.

For instance, PEG-based chains are amphiphilic, non-immunogenic and not susceptible to cleavage by proteolytic enzymes. Preparations of materials that have been modified by PEG or PEG-based chains, have reduced immunogenicity and antigenicity. See for example, Abuchowski, et al, J: Biol. Chem. (1977) 252(11):3578-3581; Tsutsumi, et al, Jpn. J Cancer Res. (1994) 85:9-12; Poly(ethylene glycol) Chemistry and Biological Applications, ACS Symposium Series 680, J. M. Harris and S. Zalipsky, Eds., American Chemical Society, 1997; and Poly(ethylene glycol) Chemistry, Biotechnical and Biomedical Applications, Topics in Applied Chemistry, J. M. Harris, Ed., Plenum Press, New York, N.Y., 1992). PEG also serves to increase the molecular size of the material, to which it is attached, thereby increasing its biological half-life. These beneficial properties of the PEG-modified materials make them very useful in a variety of therapeutic applications. Accordingly, this invention also contemplates improving the pharmacokinetics of the polypeptides of the invention, by the modification or “PEGylation” of the polypeptides at sites that are likely to permit the proteins to retain their intrinsic biological activity. Such sites include, but are not limited to, the C-terminus of the polypeptide. The grafting of PEG chains or PEG-based chains onto proteins is known. See for example, Zalipsky, U.S. Pat. No. 5,122,614, which describes PEG that is converted into its N-succinimide carbonate derivative. Also known are PEG chains modified with reactive groups to facilitate grafting onto proteins. See for example, Harris, U.S. Pat. No. 5,739,208, which describes a PEG derivative that is activated with a sulfone moiety for selective attachment to thiol moieties on molecules and surfaces and Harris, et al., U.S. Pat. No. 5,672,662, which discloses active esters of PEG acids that have a single propionic or butanoic acid moiety. This area is extensively reviewed in Zalipsky, Bioconjugate Chemistry (1995) 6:150-165. Besides use of PEG, Wright, EP 0 605 963 A2 describes linking reagents that contain water soluble polymers that form a hydrazone linkage with an aldehyde group on a protein. All of the aforementioned references are incorporated herein by reference.

B. The Polymer-Modification of the Bioactive Synthetic Chemokines of the Present Invention

The present invention additionally relates to bioactive synthetic chemokines that have been modified by a polymer adduct, and to methods for their production and use. The invention particularly relates to such synthetic chemokines that posess changes at one or more residues of their N-terminal region. Modified chemokines that possess such changes are typically potent antagonists of their corresponding receptors. In general, the most potent changes found to date have been hydrophobic in nature, e.g., Methionine- (Met-), aminooxypentane- (AOP-) and nonanoyl-(NNY- ) modifications to the N-terminus of each major category of chemokines; further increases in potency can be made through replacement of wild type amino acids within the N-terminal region with amino acids or hydrophobic derivatives (e.g., CC chemokines such as Rantes, MCP, and MIP and CXC chemokines such as SDF1 and IL-8) (See, U.S. Patent Application Ser. No. 60/217,683 (which is incorporated by reference in its entirety). Moreover, introduction of hydrophobic modifications to the C-terminal region of chemokines increases potency even further, which supports the concept that hydrophobicity of both the N- and C-terminal regions influence potency (See, U.S. Patent Application Ser. No. 60/217,683). Thus attachment of a water-soluble polymer would ordinarily be expected to significantly reduce or even destroy the desired antagonistic activity of such chemokines, particularly downmodulation of the corresponding receptor to which the polymer-modified chemokine binds, given that downmodulation can be enhanced by increasing the hydrophobic nature of the N- and C-termini of a given chemokine. Zalipsky, S. (Bioconjugate Chemistry (1995) 6:150-165), Mehvar, R. (J Pharm. Pharmaceut. Sci. (2000) 3(1):125-136) and Monfardini et al. (Bioconjugate Chem. (1998) 9:418450) review various water-soluble polymers, their attachment to peptide and proteins, and biological effects.

One aspect of the present invention derives from the finding that attachment of water-soluble polymers to bioactive synthetic chemokines does affect potency as the above general theory prescribes, but that potency and receptor-specificity can be controlled depending on the site of attachment, the nature of the polymer that is attached, and the precursor chemokine targeted for polymer-modification. The present invention also derives from the finding that the efficacy of a bioactive synthetic chemokine can be increased by systematically improving the balance between a desired antagonistic property and circulating half-life. This optimization involves a three-component process, yielding synthetic chemokines with ever increasing potency and circulating half-life when the components are successively combined. This process is generally applicable to all chemokines, with each component also having general application in the generation of bioactive synthetic chemokines having a water-soluble polymer attached thereto, and having the in vitro bioactivity of downmodulating a corresponding chemokine receptor to which the synthetic chemokine binds. The term “downmodulating a chemokine receptor,” as used herein, is intended to denote causing a reduction in normal basal activity of a chemokine receptor characterized by one or more of calcium signaling, leukocyte chemotaxis and viral infection, following its binding to a chemokine or chemokine analog; may include prolonged or enhanced removal of the receptor from a cell surface.

The first component relates to the precision modification of a precursor bioactive synthetic chemokine with a water-soluble polymer of interest at one or more sites that retains the in vitro bioactivity of the precursor bioactive synthetic chemokine. In vitro bioactivity is a good criteria for assessing function and standard assays for individual chemokines are well known for this purpose (See e.g., Cytokine Reference, Vol. 1, Ligands, A compendium of cytokines and other mediators of host defense, Eds. J. J. Oppendheim and M. Feldmann, Acedemic Press, 2001; and Cytokine Reference, Vol. 2, Receptors, A compendium of cytokines and other mediators of host defense, Eds. J. J. Oppendheim and M. Feldmann, Acedemic Press, 2001). Preferred attachment sites are selected from a residue of the precursor chemokine corresponding to a C-terminal site, an aggregation site, a glycosylation site, and a glycosaminoglycan (“GAG”) binding site. Kuschert et al. (Biochem. (1999) 38:12959-12968), Koppman et al. (J. Immunol. (1999) 163:2120-2127) and Proudfoot et al. (J. Biol. Chem. (2001) 276(14):10620-10626) report on GAG binding and chemokines.

The second component relates to the nature of the polymer being attached, which preferably has a formula U-B-Polymer-J, where U is a residue of a functional group attached to the protein, B is a branching core having three or more arms and may be present or absent, Polymer is a substantially non-antigenic water-soluble polymer having a molecular weight equivalent to or greater than 1000 Daltons (“Da”), and J is a pedant group that has a desired net charge under physiological conditions selected from negative, neutral or positive.

An unexpected finding with respect to the polymer-modified bioactive synthetic chemokines of the invention is that a water-soluble polymer construct as small as about 1000-1500 Da can be sufficient to increase the serum circulating half-life of the precursor chemokine by about 10-fold. Another unexpected finding is that a small linear polymer construct and a much larger branched polymer construct, when attached at the same site can have similar effects on potency and receptor downmodulation. Another finding is that the nature and number of pendant groups J on the polymer construct can influence downmodulation and receptor specificity of the bioactive synthetic polymer-modified chemokine.

For instance, use of branched polymer constructs having a plurality of ionizable pendant groups J can alter GAG and receptor binding by design. By way of example, synthetic chemokines that are analogs of RANTES, where a branched polymer having negatively charged groups (under physiological conditions) bias binding towards CCR5, which has a net electrostatic surface that appears neutral, as opposed to CCR1 which has a net electrostatic surface that is negative. Thus a polymer-modified bioactive synthetic Rantes analog having a water-soluble polymer comprising a plurality of negatively charged pendant J groups can preferentially interact with a receptor having a net neutral or net positive surface. Similarly, as GAGs typically exhibit a net negative charge, interaction with them can be manipulated. Of course as noted above, the attachment site of the polymer can be exploited to fine tune elements of receptor and GAG binding depending on a given end use of a bioactive synthetic polymer-modified chemokine of interest.

The third component relates to the generation of polymer-modified bioactive synthetic chemokines having optimal in vitro bioactivity characterized by downmodulation of a chemokine receptor to which it binds. This involves selection of a precursor chemokine (i.e., a natural or modified chemokine chosen for attachment of a water-soluble polymer thereto) having a potency that is about 1- to 5-fold or more, and preferably about 5- to 10-fold greater or more (as measured by EC50 in a relevant in vitro cell-based assay) than the desired potency range of the polymer-modified bioactive synthetic chemokine. Selection of such precursor chemokines has been found to yield polymer-modified bioactive synthetic chemokines that exhibit a desired balance of potency and in vivo serum circulating half-life. The term “EC50” is intended to denote the concentration of a compound that provokes a response half way between the baseline and maximum response on a dose-response (or concentration-response) curve. For example, EC50 can be a measure of potency, where a smaller EC50 for a given response to be measured represents a more potent compound compared to a compound having a higher EC50. EC50 also is commonly referred to as ED50 or IC50, and in the context of viral infection is the effective concentration that inhibits 50% of viral production, 50% of viral infectivity, or 50% of the virus-induced cytopathic effect.

It will be appreciated that synthetic chemokines are provided that can be derived from each of these components (i.e., polymer attachment site, the nature of the polymer, and selected of precursor chemoldne for polymer modification) alone or in combination. The polymer attachment sites also may overlap or be one in the same, e.g., aggregation site and GAG site are adjacent or are localized at the same site. Moreover, synthetic chemokines are provided that have two or more polymers attached thereto. For example, the invention also includes bioactive synthetic chemokines that comprise a chemokine polypeptide chain and a water-soluble polymer attached thereto at a first site selected from a GAG site and at a second site selected from an aggregation site and a C-terminal site. This aspect of the invention permits, inter alia, one to increase the molecular weight and water-solubility (thus improving circulating half-life and other desirable properties afforded by the water-soluble polymer) while eliminating less desirable properties such as aggregation and particular types of GAG binding at the sites of polymer attachment.

1. Polymer Attachment Sites

Preferred bioactive synthetic chemokines of the invention can be made by precision modification of the bioactive synthetic chemokine proteins of the present invention with a water-soluble polymer of interest at a residue of one or more sites selected from a C-terminal site, an aggregation site, a glycosylation site, and a GAG binding site. Residues at these sites can be used for attachment, provided they have a side-chain amenable for polymer attachment (i.e., the side chain of an amino acid bearing a functional group, e.g., lysine, aspartic acid, glutamic acid, cysteine, histidine, etc.). Alternatively, a residue at these sites can be replaced with a different amino acid having a side chain amenable for polymer attachment. Also, the side chains of the genetically encoded amino acids can be chemically modified for polymer attachment, or unnatural amino acids with appropriate side chain functional groups can be employed. The preferred method of attachment employs a combination of peptide synthesis and chemical ligation.

Such bioactive synthetic chemokines of the present invention are preferably synthesized by the condensation of amino acid residues. Such amino acid residues may be the nucleic acid encoded, ribosomally installed amino acids: alanine, arginine, asparagine, aspartate, cysteine, glutamate, glutamine, glycine, histidine, isoleucine, leucine, lysine, methionine, phenylalanine, proline, serine, threonine, tryptophan, tyrosine and valine. In one embodiment, the amino acid sequence selected for a particular position of a bioactive synthetic chemokine of the present invention would be the native, or naturally present amino acid residue found at that position in the sequence of that protein. In an alternative embodiment, the selected amino acid sequence will be composed of, or constructed from, polypeptide fragments that may contain an N-terminal cysteine residue in place of the amino acid residues present in the native or natural sequence of that protein. The inclusion of such a residue permits the polypeptide to be ligated to another polypeptide (modified to contain a carboxy thioester group) using the principles and methods of native chemical ligation, peptide synthesis and/or convergent synthesis (see, U.S. Patent Application Ser. Nos. 60/231,339, 60/236,377 and 09/097,094, all herein incorporated by reference), which principles are preferably employed to synthesize the bioactive synthetic chemokines of the present invention.

In a further embodiment, the synthetic bioactive proteins of the present invention may contain “irregular” amino acid residues. As used herein, the term “irregular amino acid residues is intended to refer to amino acids that are not encoded by RNA and are not ribosomally installed. In this regard, the present invention permits wide selectability and flexibility in the design and/or construction of synthetic bioactive proteins. Examples of non-ribosomally installed amino acids that may be used in accordance with a present invention include: D-amino acids, β-amino acids, pseudo-glutamate, γ-aminobutyrate, ornithine, homocysteine, N-substituted amino acids (R. Simon et al., Proc. Natl. Acad. Sci. U.S.A. (1992) 89: 9367-71; WO 91/19735 (Bartlett et al.), U.S. Pat. No. 5,646,285 (Baindur), α-aminomethyleneoxy acetic acids (an amino acid-Gly dipeptide isostere), and α-aminooxy acids, etc. Peptide analogs containing thioamide, vinylogous amide, hydrazino, methyleneoxy, thiomethylene, phosphonamides, oxyamide, hydroxyethylene, reduced amide and substituted reduced amide isosteres and α-sulfonamide(s) may be employed.

In particular, the use of pseudo-native chemical ligation is advantageous since the R chain modification permits attachment of polymer adducts to the synthesized protein. Likewise, N-terminal Na-substituted 2 or 3 carbon chain alkyl or aryl thiol amino acids may be employed. Such residues (where present at the end terminus or polypeptide) can be advantageously used to ligate that polypeptide to a polypeptide having a carboxy thioester moiety, in accordance with the methods of extended native chemical ligation described herein.

Peptide synthesis is preferably based on the “Merrifield”-chemistry stepwise solid phase peptide synthesis protocol developed in the early 1960's, using standard automated peptide synthesizers. The peptide ligation step may employ solid or solution phase ligation strategies. Chemical ligation involves the formation of a selective covalent linkage between a first chemical component and a second chemical component. Unique, mutually reactive, functional groups present on the first and second components can be used to render the ligation reaction chemoselective. For example, the chemical ligation of peptides and polypeptides involves the chemoselective reaction of peptide or polypeptide segments bearing compatible unique, mutually-reactive, C-terminal and N-terminal amino acid residues.

In one embodiment, all of the amino acid residues of the synthetic bioactive protein may be joined together by a peptide bond (i.e., an amide bond). Alternatively, two amino acid residues (or the C-terminal and N-terminal residues of two polypeptides) may be linked to one another by a non-amide bond (such as a thioester bond, an oxime bond, a thioether bond, a directed disulfide bond, a thiozolidine bond, hydrazone forming ligation, oxazolidine forming ligation, etc.) (Schnölzer, M. and Kent, S. B. H., Science (1992) 256:221-225; Rose, K., J. Amer. Chem Soc. (1994) 116:30-33; Englebretsen, D. R. et al., Tetrahedron Lett. 36:8871-8874; Baca, M. et al., J. Amer. Chem Soc. (1995) 117:1881-1887; Liu, C. F. et al., J. Amer. Chem Soc. (1994) 116:4149-4153; Liu, C. F. et al., J. Amer. Chem Soc. (1996) 118:307-312; Dawson, P. E. et al. (1994) Science 266:776-779; Gaertner, et al., Bioconj. Chem. (1994) 5(4):333-338; Zhang, et al., Proc. Natl. Acad. Sci. (1998) 95(16):9184-9189; Tam, et al., WO 95/00846; U.S. Pat. No. 5,589,356) orby other methods (Yan, L. Z. and Dawson, P. E., “Synthesis of Peptides and Proteins without Cysteine Residues by Native Chemical Ligation Combined with Desulfurization,” J. Am. Chem. Soc. 2001, 123, 526-533, herein incorporated by reference; Gieselnan et al., Org. Lett. 2001 3(9):1331-1334; Saxon, E. et al., “Traceless” Staudinger Ligation for the Chemoselective Synthesis of Amide Bonds. Org. Lett. 2000, 2, 2141-2143. The invention thus permits a variety of peptide bond modifications, surrogates and isosteric replacements to be exploited in the preparation of bioactive proteins.

Where the ligation involves the joining of a polypeptide that possesses an N-terminal cysteine residue, the procedure of native chemical ligation is preferably employed (Dawson, et al., Science (1994) 266:776-779; Kent, et al., WO 96/34878; Kent, et al., WO 98/28434)). This methodology has proven a robust methodology for generating a native amide bond at the ligation site. Native chemical ligation involves a chemoselective reaction between a first peptide or polypeptide segment having a C-terminal α-carboxythioester moiety and a second peptide or polypeptide having an N-terminal cysteine residue. A thiol exchange reaction yields an initial thioester-linked intermediate, which spontaneously rearranges to give a native amide bond at the ligation site while regenerating the cysteine side chain thiol. In many instances, the sequence of the natural protein will comprise suitably placed cysteine residues such that polypeptide fragments having an N-terminal cysteine residue may be synthesized and used in a native chemical ligation reaction. In other instances, the peptide synthesis can be conducted so as to introduce cysteine residues into a polypeptide for this purpose.

However, where it is either inconvenient or undesirable to modify a natural protein sequence so as to introduce a cysteine residue at the N-terminus of a polypeptide, the method of native chemical ligation may be extended using polypeptides whose N-terminus has been modified to contain an N-substituted, and preferably, Na-substituted, 2 or 3 carbon chain amino allyl or aryl thiol. Such “extended native chemical ligation” is described in U.S. Patent Application Ser. Nos. 60/231,339, and 60/236,377, herein incorporated by reference.

In brief, the method involves ligating a first component comprising a carboxylthioester, and more preferably, an α-carboxylthioester with a second component comprising an acid stable N-substituted, and preferably, Nα-substituted, 2 or 3 carbon chain amino alkyl or aryl thiol. Chemoselective reaction between the carboxythioester of the first component and the thiol of the N-substituted 2 or 3 carbon chain alkyl or aryl thiol of the second component proceeds through a thioester-linked intermediate, and resolves into an initial ligation product. More specifically, the thiol exchange occurring between the COSR thioester component and the amino alkyl thiol component generates a thioester-linked intermediate ligation product that after spontaneous rearrangement generates an amide-linked first ligation product through a 5-membered or 6-membered ring intermediate depending upon whether the amino alkyl thiol component has formula I or II, respectively:
J1-C(O)—N(C1(R1)—C2-SH)—J2 I
J1-C(O)—N(C1(R1)-C2(R2)-C3(R3)-SH)—J2 II
where J1 is a peptide or polypeptide having one or more optionally protected amino acid side chains, or a moiety of such peptide or polypeptide, a polymer, a dye, a suitably functionalized surface, a linker or detectable marker, or any other chemical moiety compatible with chemical peptide synthesis or extended native chemical ligation; R1, R2 and R3 are independently H or an electron donating group conjugated to C1; with the proviso that at least one of R1, R2 and R3 comprises an electron donating group conjugated to C1; and J2 is a peptide or polypeptide having one or more optionally protected amino acid side chains, or a moiety of such peptide or polypeptide, a polymer, a dye, a suitably functionalized surface, a linker or detectable marker; or any other chemical moiety compatible with chemical peptide synthesis or extended native chemical ligation.

The N-substituted 2 or 3 carbon chain alkyl or aryl thiol [HS—C2-C1(R1)-] or [HS—(C3(R3)-C2(R2)-C1(R1)-] at the ligation site is amenable to being removed, under peptide-compatible conditions, without damage to the product, to generate a final ligation product of formula III, having a native amide bond at the ligation site:
J1-C(O)—HN-J2 III
where J1, J2, R1, R2, and R3 are as defined above.

The R1, R2 and R3 groups are selected to facilitate cleavage of the N—C1 bond under peptide compatible cleavage conditions. For example, electron donating groups, particularly if conjugated to C1, can be used to form a resonance stabilized cation at C1 that facilitates cleavage. The chemical ligation reaction preferably includes as an excipient a thiol catalyst, and is carried out around neutral pH conditions in aqueous or mixed organic-aqueous conditions. Chemical ligation of the first and second components may proceed through a five or six member ring that undergoes spontaneous rearrangement to yield an N-substituted amide linked ligation product. Where the first and second components are peptides or polypeptides, the N-substituted amide linked ligation product has formula IV or V:
J1-C(O)—Nα(C1(R1)-C2-HS)—CH(Z2)-C(O)-J2 IV
J1-C(O)—Nα(C1 (R1)-C2(R2)-C3(R3)-HS)—CH(Z2)-C(O)-J2 V
where J1, J2 and R1, R2, R3 are as defined above and Z2 is an amino acid side chain or a derivative of such side chain.

The conjugated electron donating groups R1, R2 or R3 of the N-substituted amide bonded ligation product facilitate cleavage of the N—C1 bond and removal of the 2 or 3 carbon chain alkyl or aryl thiol from the N-substituted amide-linked ligation product. Removal of the alkyl or aryl thiol chain of the N under peptide-compatible cleavage conditions generates a ligation product having a native amide bond at the ligation site. Where the first and second components are peptides or polypeptides, the ligation product will have the formula:
J1-CONαH—CH(Z2)—C(O)-J2 VI

An advantage of attachment to a C-terminal site is that it can minimize potential loss in activity due to maximal separation from known functional regions of most chemokines. An advantage of attachment to chemokines possessing one or more aggregation sites is that the water-soluble polymer can disrupt aggregation while minimizing potential loss of activity, and improve handling/formulation and in vivo efficacy. Attachment to glycosylation sites has the advantage of mimicking the positive effects of a sugar chain normally found at that site, while minimizing potential loss in activity by attachment of synthetic polymer at a natural polymer attachment site. Another advantage of polymer-modification at a glycosylation site is that a polymer can be employed that comprises a cleavable linker so as to provide the synthetic chemokine in prodrug form, particularly where glycosylation sites occur at or near the N-terminal pharmacophore region. An advantage of attachment to a GAG binding site is that the polymer can be exploited to disrupt GAG binding at that site and in some instances aggregation where such sites overlap while minimizing potential loss of activity, improve circulating half-life by reducing binding to undesirable surfaces bearing GAG moieties, and improve handling/formulation and in vivo efficacy. Alternatively, depending on the water-soluble employed for attachment, GAG binding can be enhanced, or binding of particular types of GAG can be enhanced. Of course depending on the target chemokine for modification, a site for polymer attachment may be preferred over another, for instance, while all chemokines have a C-terminal region and one or more GAG binding sites, not all chemokines appear to possess aggregation and glycosylation sites.

Advantageously, the attachment of a water-soluble polymer will be through a biodegradable linker, especially at the N-terminal region of a protein. Such modification acts to provide the protein in a precursor (or “pro-drug”) form, that, upon degradation of the linker releases the protein without polymer modiication. Attachment through, and use of biodegradable linkages, such as ester linkages are well known, and are described in more detail below.

a. C-terminal Sites

Preferred bioactive synthetic chemokines of the invention have a water-soluble polymer attached at a C-terminal site. By “C-terminal site” is intended a residue of a chemokine polypeptide chain that is C-terminal to the C-terminal α-helix of a chemokine. This includes the pendant C-terminal residue bearing a free α-carboxylate and residues adjacent thereto. A prominent secondary structural feature of all chemokines is the triple-stranded anti-parallel β sheet that forms a sheet floor for the hydrophobic C-terminal α-helix to lay across. Thus the C-terminal α-helix is a consistent feature of chemokines that is readily identifiable, for instance by homology modeling and comparison, for example, by comparing primary sequences and/or three-dimensional structures of known chemokines, or predicted structures through molecular replacement and energy minimization algorithms (See e.g., Cytokine Reference, Vol. 1, Ligands, A compendium of cytokines and other mediators of host defense, Eds. J. J. Oppendheim and M. Feldmann, Acedemic Press, 2001)).

A preferred C-terminal site is one that is adjacent to the pendant C-terminal residue of a precursor chemokine targeted for polymer modification. An example is for analogs of Rantes. The C-terminal residue of wild type Rantes (1-68) is the serine at position 68 (i.e., Ser68 or S68). Attachment of a water-soluble polymer to a residue adjacent to the pendant C-terminal residue of a precursor chemokine such as NNY-Rantes (2-68) would include the methionine at position 67 (M67) as well as linker moieties attached to residues corresponding to M67 and/or S68. For instance, addition of a linker or spacer component having a functional side chain for coupling a water-soluble polymer to the C-terminus of a chemokine of interest minimizes the potential impact on function of the original C-terminal group at or near that position. For example, addition of linker to position S68 of Rantes, such as the dipeptide linker Lys69-Leu70 gives Rantes ((1-68)-(K69-L70)) and provides a functional coupling group for polymer attachment to the epsilon nitrogen on the side chain of linker moiety Lys69, and an amino acid with a free α-carboxylate at the newly generated C-terminus. Addition of a spacer or linker to a residue corresponding to position 67 (e.g., replacement of Met67 with Lys67, and attachment of linker to the epsilon side-chain amino group) also is possible. Both have been found to retain bioactivity, and this design appears to be of general application to other chemokines. For instance, analogous modifications of SDF-1α or SDF-1β can generate polymer-modified SDF-1 analogs that retain desired bioactivity. In yet another example, when the synthetic chemokine is an analog of MCP-1 or Eotaxin, and where a water-soluble polymer is attached at a residue that is adjacent to C-terminus, the polymer attachment site comprises an amino acid corresponding to D68 of MCP-1 or D66 of Eotaxin, respectively. Here again residues at a C-terminal site that are adjacent to these positions can be exploited for polymer attachment, such as K69 of MCP-1 or K68 of Eotaxin. Moreover, addition of a linker or spacer moiety permits more flexibility in the addition of other moieties at or near the C-terminus, such as a hydrophobic moiety like a lipid or polycyclic. Accordingly, additional preferred polymer-modified bioactive synthetic chemokines of the invention comprise a water-soluble polymer attached at a C-terminal site through the side chain of a linker or spacer moiety.

b. Aggregation Sites

Of particular interest are synthetic chemokines having a water-soluble polymer attached to one or more aggregation sites. By “aggregation site” is intended residue(s) causing self-association of protein monomers. Most chemokines have the potential to form homodimers, with many capable of forming tetramers, and some even larger multimers (Cytokine Reference, Vol. 1, Ligands, A compendium of cytokines and other mediators of host defense, Eds. J. J. Oppendheim and M. Feldmann, Acedemic Press, 2001). Aggregation sites of chemokines typically are found in chemokines capable of forming dimer and multimer complexes at high concentrations (Cytokine Reference, Vol. 1, Ligands, A compendium of cytokines and other mediators of host defense, Eds. J. J. Oppendheim and M. Feldmann, Acedemic Press, 2001). For instance, chemokines such as Rantes and MIP1β form aggregates through self-association of monomers at high concentration, whereas chemokines such as IL-8, SDF1α and vMIP do not have this tendency to any significant degree. Aggregation sites are identifiable via numerous well known methods, such homology modeling, alanine scanning and comparison of active compounds at increasing concentrations in solution and monitoring for aggregation, self-association using various techniques known in the art. (See e.g., Czaplewski et al., J. Biol. Chem. (1999) 274(23):16077-16084; Czaplewski et al., “Engineering, Biology, and Clinical Development of hMIP-1α” (1999) In: Chemokines in Disease: Biology and Clinical Research, Ed., C. A. Herbert, Humana Press Inc., Totwa, N.J.; Trkola et al., J. Virol. (1999) 73(8):6370-6379; Appay et al., J. Biol. Chem. (1999) 274(39):27505-27512; Hunter et al., Blood (1995) 86(12):4400-4408; Lord et al., Blood (1995) 85(12):3412-3415; Lord et al., Brit. J. Cancer (1996) 74:1017-1022). And as noted above, the aggregation sites of many chemokines are known, and the techniques for identifying putative aggregation sites are well known (See also, Cytokine Reference, Vol. 1, Ligands, A compendium of cytokines and other mediators of host defense, Eds. J. J. Oppendheim and M. Feldmann, Acedemic Press, 2001). Thus aggregation sites of chemokines can be identified from published information, homology modeling, through screening, or a combinations of each. Thus the examples presented herein are illustrative of the invention, and thus are not intended to limit the invention.

By way of example, aggregation sites have been identified in numerous chemokines through various techniques, such as those described above. For instance, wild type Rantes posses at least two residues involved in aggregation: the glutamic acids at residue positions 26 and 66 (i.e., Glu26 and Glu66; or E26 and E66). Thus when the synthetic chemokine is an analog of Rantes, and where a water-soluble polymer is attached at an aggregation site, the aggregation site can comprise an amino acid corresponding to a residue of Rantes selected from E26 and E66. For instance, we have attached a water-soluble polymer that is adjacent to E66 of Rantes, namely, at a position corresponding to methionine 67 (i.e., Met67 or M67). This polymer modification disrupts aggregation and improves the overall handling properties, presumably due to the large hydrophobic shell contributed by the polymer that disrupts aggregation properties of the adjacent glutamic acid residue. Thus, in the context of the present invention, the aggregation site in Rantes corresponds not only to E26 and E66, but amino acids that are adjacent thereto. Thus the aggregation site in Rantes comprises an amino acid corresponding to a residue of Rantes selected from E26 and E66, including M67.

As another example, when the synthetic chemokine is an analog of MIP1α, and where a water-soluble polymer is attached at an aggregation site thereof, the aggregation site comprises an amino acid corresponding to a residue of MIP1α selected from D26 and E66. Similarly, when the synthetic chemokine is an analog of MIP1β, and where a water-soluble polymer is attached at an aggregation site thereof, such an aggregation site can comprise an amino acid corresponding to a residue of MIP 1β selected from positions D27 and E67. In yet another example, when the synthetic chemokine is an analog of MCP-1 or Eotaxin, and where a water-soluble polymer is attached at an aggregation site, the aggregation site comprises an amino acid corresponding to residue P8 and D68 of MCP-1 or D66 of Eotaxin, respectively. Here again residues adjacent to these positions can be exploited for polymer attachment in order to disrupt aggregation, such as K69 of MCP-1 or K68 of Eotaxin, and thus is embodied in bioactive synthetic chemokines of the invention having a water-soluble polymer attached at an aggregation site thereof. As can be appreciated, aggregation sites are readily identifiable and are preferred sites for polymer-modification, and can be selected for preferential attachment through routine screening for the desired bioactivity.

In a more preferred embodiment, the water-soluble polymer is attached at an aggregation site located at the C-terminal region of the chemokine of interest. An even more preferred aggregation site for polymer attachment is one that is C-terminal to the C-terminal α-helix of chemokines, such as E66 and M67 of Rantes, E66 of MIP1α, E67 of MIP1β, D68 of MCP-1, or D66 of Eotaxin. Accordingly, preferred bioactive synthetic chemokines of the invention include those having a water-soluble polymer attached at a C-terminal aggregation site that is C-terminal to the C-terminal α-helix thereof. The most preferred bioactive synthetic chemokines of this aspect of the invention are those having a water-soluble polymer attached at a C-terminal aggregation site that is C-terminal to the C-terminal α-helix thereof and that is adjacent to the pendant C-terminal residue of a precursor chemokine.

C. Glycosylation Sites

In another preferred embodiment, bioactive synthetic chemokines of the present invention having a water-soluble polymer attached at a glycosylation site are provided. By “glycosylation site” is intended residues coding for enzymatic attachment of carbohydrate (oligosaccharide) chain, such as N-linked and O-linked glycosylation sites. More preferred glycosylation sites are those that occur at the C-terminal region of a chemokine. The N-linked glycosylation sites are the most preferred. The glycosylation sites can be natural site or engineered into target protein. They can be identified in wild type molecule by analytical tests for the presence of saccharide and their attachment site, homology comparison, or by scanning consensus sequences and/or structures against gene and protein databases. An advantage of engineering a glycosylation site into a target protein is that additional preferred sites for polymer attachment can be identified, provided that the engineered sites do not disrupt the desired bioactivity of interest.

In particular, most proteins synthesized by the ribosomes of the rough endoplasmic reticulum contain short chains of carbohydrates (oligosaccharides) and are called glycoproteins (See, e.g., Van den Steen et al., Critical Rev. Biochem. Mol. Biol. (1998) 33(3):151-208). And many chemokines are known to be glycosylated, or contain consensus sites for N-linked and/or O-linked glycosylation. Indeed, many naturally produced chemokines are heavily glycosylated. This makes glycosylation sites of chemokines of particular interest for polymer-modification. For instance, glycoproteins are responsible for many important properties of the protein (e.g., p1 molecular size, solubility, stability, structure, and electrostatic surface charge) and eukaryotic cell surface (e.g. cell-cell recognition and adhesion, cell-surface charge protection against “wear and tear,” blood group specific antigens, virus, bacteria and protozoan receptors, transplantation (histocompatibility) antigens, ion channels and many others). Thus attachment of water-soluble polymers at such sites can be exploited to mimic advantageous properties (e.g., pI, size, solubility, stability, reduced antigenicity, prolonged circulating half-life), while eliminating others (e.g., heterogeneity, saccharide-mediated clearance and unwanted adhesion) to generate a drug compound having improved properties.

Moreover, attachment of a water-soluble polymer at one or more glycosylation sites should in general reduce the impact on bioactivity, and possibly improve it since nature has selected such sites for attachment of large water-soluble polymers. The carbohydrate content of glycoproteins (frequently 50% or more of the total weight) is covalently attached to the polypeptide as oligosaccharide side chains typically containing 4 to 15 sugars. Several side chains may occur on the same polypeptide and the chains may be branched. And we have found that glycosylation sites are relatively good indicators of sites amenable to polymer attachment, particularly branched water-soluble polymers of relatively high molecular weight having pendant charge groups that mimic the net charge contribution of oligosaccharide chains attached through glycosylation.

In identifying glycosylation sites, the oligosaccharides of glycoproteins are of two main types: O-linked and N-linked. O-linked oligosaccahrides are commonly attached to the protein via O-glycosidic bonds to the OH groups of serine and threonine side chains. N-linked oligosaccharides are linked to the protein via N-glycosidic bonds to the NH₂ groups of asparagine side chains where the asparagine occurs in the sequence where X is any amino acid except proline and aspartate.

N-Glycosylated carbohydrate chains are bound to the Asn residue in Asn-X-Ser/Thr (X being any amino acid other than Pro) in polypeptides, as mentioned above. However, many proteins contain an unglycosylated Asn-X-Ser/Thr sequence or sequences and the presence of this sequence does not always result in addition of a carbohydrate chain thereto. However, the presence or absence of N-glycosylation can be readily tested (Biller, M. et al., J Virol Methods 1998 December;76(1-2):87-100; Taverna, M. et al., J Biotechnol 1999 Feb. 5;68(1):37-48; Friedman, Y. et al., Anal Biochem 1995 Jul. 1;228(2):221-5). On the other hand, O-glycosylated carbohydrate chains are bound to the Ser or Thr residue in polypeptides, but unlike the case of the N-glycosylation there is no rule on the amino acid sequence required for glycosylation. It is known, however, that the tendency toward glycosylation increases when Pro occurs in the vicinity, for example in the sequences Pro-Thr/Ser, Thr/Ser-Pro and Thr/Ser-X1-3-Pro (X being any amino acid) (Takahashi et al.: Proc. Natl. Acad. Sci. USA (1984) 81:2021). Here again O-linked glycosylation can be readily tested.

Many chemokines are known to be glycosylated, or to contain putative glycosylation sites. (See, e.g., Cytokine Reference, Vol. 1, Ligands, A compendium of cytokines and other mediators of host defense, Eds. J. J. Oppendheim and M. Feldmann, Acedemic Press, 2001). Thus glycosylation sites of chemokines can be identified from published information. For predicted sites, a preferred way of confirming N— and/or O-linked glycosylation sites is through homology comparisons (e.g., using database and pattern matching systems suitable for this purpose, see, e.g., Xiang, Y. et al., Virology 1999 May 10;257(2):297-302; Hoops, T. C. et al., J Biol Chem 1991 Mar. 5;266(7):4257-63); Apweiler, R. et al., Biochim Biophys Acta 1999 Dec. 6; 1473(1):4-8), followed by testing a precursor chemokine in a yeast or mammalian cell expression system, and conducting analytical tests for characterizing saccharide content and attachment sites. Alternatively, putative sites identified through homology comparisons can be modified with a water-soluble polymer of interest, and then simply screened in a relevant in vitro bioassay for the bioactivity of interest (e.g., calcium flux, chemotaxis, and/or inhibition of viral entry) following standard protocols suitable for this purpose.

By way of example, homology modeling identifies a putative O-linked glycosylation site at the serine of position 5 (i.e., Ser5 or S5) in Rantes, which is supported by other studies (Kameyoshi et al., J Exp Med (1992) 176(2):587-592) which show the glycosylated form to have chemotactic activity in the 2-10 nM range. For Rantes analogs that are antagonists, such as NNY-Rantes analogs, changing the serine at position 5 of Rantes to a charged, soluble group moiety such as Glu or Lys reduces potency, whereas introduction of an amino acid moiety such as a t-butyl alanine (tBuA) retains potency, when measured in a cell-based assay for viral infection/fusion. So for Rantes antagonists, attachment of a water-soluble polymer at a site corresponding to the S5 position in the wild type Rantes is preferably through a biodegradable liker, for example, an ester linkage to the side chain hydroxyl of serine or similar group, which would provide the Rantes antagonist compound substantially in prodrug form. Degradation and release of the Rantes analog from the water-soluble polymer in an in vivo setting then generates the active form. Attachment through, and use of biodegradable linkages, such as ester linkages are well known, and are described in more detail below.

A similar situation has been found for other chemokines such as MIP-1α and MIP-1β, both of which have one or two potential O-linked glycosylation sites. For example, MIP1α has a predicted glycosylation site that comprises an amino acid corresponding to residue T7, and for MIP1β where a predicted glycosylation site that comprises an amino acid corresponding to residue S5. These two examples are analogous to Rantes in that O-linked glycosylation sites are predicted, and thus for antagonist analogs of MIP the preferred attachment would be through a biodegradable linkage so as to permit formation of the active form following in vivo release of the polymer.

Many other chemokines have glycosylation sites, and can be synthesized and modified with one or more water-soluble polymers in accordance with the present invention. Thus the examples presented herein are illustrative of the invention, and thus are not intended to limit the invention. For example, lyphotactin is a 93-residue chemokine containing eight sites of O-linked glycosylation. This compound has been synthesized using the native chemical ligation, with a single GalNAc residue incorporated at each glycosylation site using standard Fmoc-chemistry (Marcaurelle et al., Chemistry (2001) 7(5):1129-1132). In other chemokines, such as MCP-1 the glycosylation site comprises an amino acid corresponding to residue T71 of MCP-1. Although a canonical N-glycosylation sequence is present in MCP-1 at position N14, there is no detectable N-linked sugar. Rather, a small amount of sialylated O-linked carbohydrate is added to the C-terminus of the protein (Zhang et al., J. Biol. Chem. (1994) 269:15918-15924), with the predicted site being T71. The glycosylated form has been reported to be only 2- to 3-fold less potent than non-glycosylated MCP-1 in in virto monocyte chemotaxis assays (Proost et al., J. Immunology (1998) 160:4034-4041). Another glycosylated chemokine is HCC-1, which is a the only CC-chemokine known so far which circulates in nanomolar concentrations in human plasma (Richter et al., Biochemistiy (2000) 39(35):10799-10805). HCC-1 exists in various processed forms, with the full length 74 amino acid form having O-glycosylation at position 7 (Ser7) with two N-acetylneuraminic acids and the disaccharide N-acetylgalactosamine galactose.

In a more preferred embodiment, the water-soluble polymer is attached at a glycosylation site located at the C-terminal region of the chemokine of interest. The most preferred glycosylation site for polymer attachment is one that is C-terminal to the C-terminal α-helix of chemokines. For example, where the synthetic chemokine is MCP-1, a water-soluble polymer would preferably be attached at glycosylation site corresponding to residue position T71 of wild MCP-1. Accordingly, preferred bioactive synthetic chemokines of the invention include those having a water-soluble polymer attached at a C-terminal glycosylation site that is C-terminal to the C-terminal α-helix thereof.

d. GAG Binding Sites

In another preferred embodiment of the invention, provided are bioactive synthetic chemokines having a water-soluble polymer attached at a GAG binding site thereof. By “GAG binding site” is intended residues coding for GAG binding; typically residues with primary or secondary amines such as lysine and arginine, and sometimes histidine that forms a positive charge cluster on the surface of a protein. Chemokines are known to bind GAGs, including heparin, heparan sulfate, chondroitin sulfate and dermatan sulfate, which naturally occur on endothelial cell surfaces and extracellular matrix (See, e.g., Wells et al., Inflamm. Res. (1999) 48:353-3362; Lalani et al., J. Virol. (1997) 71:4356-4363; Rot, A., Eur J Immunol (1993) 23:303-306; Witt et al., Curr Biol (1994) 4:394-400; Hoogewerf et al., Biochem (1997) 36:13570-13578; Marquezini et al., Cardiology (1995) 86:143-146; Wasty et al., Diabetologia (1993) 36:316-322). Kuschert et al. (Biochem. (1999) 38:12959-12968), Koppman et al. (J. Immunol. (1999) 163:2120-2127) and Proudfoot et al. (J. Biol. Chem. (2001) 276(14):10620-10626) report on GAG binding and chemokines.

Although not essential for function, the GAG-binding capabilities of chemokines are reported to modulate receptor interactions and the haptotactic migration of receptor-bearing cells over matrix proteins and cellular faces. (See, e.g., Rot, A., Eur J Immunol (1993) 23:303-306; Witt et al., Curr Biol (1994) 4:394-400; Hoogewerfet al., Biochem (1997) 36:13570-13578; Marquezini et al., Cardiology (1995) 86:143-146; Wasty et al., Diabetologia (1993) 36:316-322; Kuschert et al., Methods Enzymol. (1997) 287: 369-378; Kuschert et al., Biochem. (1998) 37:11193-11201; Kuschert et al., Biochem. (1999) 38:12959-12968). Binding of chemokines with soluble GAGs prevents binding of the chemokines to their receptors in most cases (Kuschert et al., Biochem. (1999) 38:12959-12968). However, the interaction of chemokines with GAGs in a few instances has been reported to potentiate activity (Wbb et al., Proc. Natl. Acad. Sci. USA (1993) 90:7158-7162; and Wagner et al., Arteriosclerosis (1989) 9:21-32)). Therefore, synthetic chemokines having a water-soluble polymer attached to a GAG binding site thereof can be exploited to modulate biological function, either by reducing GAG binding or biasing the binding of certain GAGs depending on the site chosen for attachment and its intended end use. Accordingly, a preferred embodiment of the invention is directed to bioactive synthetic chemokines having a water-soluble polymer attached thereto at a GAG binding site, and having the in vitro bioactivity of downmodulating a chemokine receptor to which it binds.

As noted above, all known chemokines are able to bind heparin, although with varying affinities, and thus all have GAG binding sites. In selecting a GAG site for polymer modification, many different techniques are suitable for this purpose. For instance, the BBXB and BBBXXB motifs, where B represents a basic residue, have been shown to be a common heparin-binding motif for several proteins, including several chemokines. (See. e.g., Cardin et al., Arteriosclerosis (1989) 9:21-32; Hileman et al., Bioessays (1998) 20(2):156-167; and Proudfoot et al., J. Biol. Chem. (2001) 276(14):10620-10626). However, GAG binding sites are not restricted to the BBAB or BBBXXB motifs. For example, the GAG binding sites in Rantes (⁴⁴RKNR⁴⁷ and ⁵⁵KKWVR⁵⁹), SDF-1 (²⁴ KHL²⁷(27), MIP-1α (⁴⁵KRSR⁴⁸) and MIP-1β (⁴⁵KRSK⁴⁸) have a BBXB motif, whereas the main GAG-binding residues in IL-8 (Lys20, Lys64, and Arg68), and MCP-1 (Lys59 and Arg66) are spatially separate, but form a basic charge cluster on the protein surface. Thus the basic charge of these ligands accounts for their heparin-binding properties.

GAG sites are also identifiable by alanine scanning of basic residues (e.g., Lys, His and Arg), NMR and comparison of active compounds in GAG/heparin binding assays, as well as NMR studies adapted for this purpose (See, e.g., Proudfoot et al., J. Biol. Chem. (2001) 276(14):10620-10626; Trkola et al., J. Virol. (1999) 73(8):6370-6379; Appay et al., J. Biol. Chem. (1999) 274(39):27505-27512; Hunter et al., Blood (1995) 86(12):4400-4408; Lord et al., Blood (1995) 85(12):3412-3415; Lord et al., Brit. J. Cancer (1996) 74:1017-1022). And as noted above, the GAG binding sites of many chemokines are known, and the techniques for identifying putative GAG sites are well known (See also, Cytokine Reference, Vol. 1, Ligands, A compendium of cytokines and other mediators of host defense, Eds. J. J. Oppendheim and M. Feldmann, Acedemic Press, 2001). Thus GAG binding sites of chemokines can be identified from published information, homology modeling, through screening, or a combinations of each. Moreover, as at least one of the side chain of a residue involved in GAG binding will be located on the surface of the molecule and away from the N-terminal pharmacophore region, these sites should be generally amenable to attachment of a water-soluble polymer.

By way of example, wild type Rantes posses at least two major GAG binding sites, which comprises an amino acid corresponding to a residue of Rantes selected from Lys44, Lys45, Arg47, Lys55, Lys56, and Arg59 (i.e., K44, K45, R47, K55, K56, and R59). Thus when the synthetic chemokine is an analog of Rantes, and where a water-soluble polymer is attached at GAG binding site thereof, the GAG binding site comprises an amino acid corresponding to a residue of Rantes selected from K44, K45, R47, K55, K56, and R59. In a preferred embodiment, a water-soluble polymer is attached at a position corresponding to a residue of Rantes selected from K44, K45, R47, with position K45 being more preferred. In this embodiment, polymer attachment at position 45 has the benefit of reducing binding of the synthetic Rantes chemokine analog to the CCR1 receptor, while substantially retaining binding to CCR5.

As another example, when the synthetic chemokine is an analog of MIP1α, and where a water-soluble polymer is attached at an GAG binding site thereof, the GAG binding site comprises an amino acid corresponding to a residue of MIP1α selected from R17, R45, and R47. Similarly, when the synthetic chemokine is an analog of MIP1β, and where a water-soluble polymer is attached at an GAG binding site thereof, such an aggregation site can comprise an amino acid corresponding to a residue of MIP1β selected from positions R18, R45, and R46. In these two examples, the preferred site for polymer attachment is at R45 of MIP1-α and R46 of MIP1β. As with Rantes, these modifications are designed to bias binding of the chemokine analog to CCR5.

In a further example, when the synthetic chemokine is an analog of SDF 1-α, and where a water-soluble polymer is attached at an GAG binding site, the GAG binding site comprises an amino acid corresponding to residue K24, H25 and K27 of SDF1-α. Here again residues adjacent to these positions can be exploited for polymer attachment in order to achieve substantially the same result, such as N22 or N30 or N33, particularly N33, and thus is embodied in bioactive synthetic chemokines of the invention having a water-soluble polymer attached at an GAG binding site thereof.

In yet another example, when the synthetic chemokine is an analog of IL-8, and where the water-soluble polymer is attached at a GAG binding site, the GAG site comprises an amino acid corresponding to a residue of IL-8 selected from K20, R60, K64, K67 and R68. The preferred site of polymer attachment for a synthetic analog of L-8 corresponds to position K64 thereof. Another example is MCP-1, so that where the synthetic chemokine is an analog of MCP-1, and where a water-soluble polymer is attached at a GAG site thereof, the GAG site comprises an amino acid corresponding to a residue of MCP-1 selected from K58 and H66, with K58 being preferred.

As can be appreciated, GAG binding sites are readily identifiable and are preferred sites for polymer-modification, and can be selected for preferential attachment through routine screening for the desired bioactivity. Bioassays suitable for this purpose are well known and replete in the literature (Cytokine Reference, Vol. 1, Ligands, A compendium of cytokines and other mediators of host defense, Eds. J. J. Oppendheim and M. Feldmann, Acedemic Press, 2001).

2. Water-Soluble Polymers

Preferably, the polymer modification of the invention that is to be attached to a bioactive synthetic chemokine of the invention will be a water-soluble polymer. The term “water-soluble polymer” as used herein is intended to denote a substantially non-antigenic polymer construct that is soluble in water. Examples of biological water-soluble polymers include, but are not limited to (a) dextran, dextran sulfate, carboxymethyl dextrin; (b) glycosaminoglycans such as heparin, heparan sulfate, chondroitin sulfate and dermatan sulfate; (c) hyaloronin and hyaluronic acid; (d); polylactide and oligolactyl-acrylate; (e) cellulose, methylcellulose and carboxymethyl cellulose; (f) collagen; (g) gelatin; (h) alginate; and (i) starches.

The water-soluble polymers of the present invention may be linear, branched, or star-shaped, or a mixture of such conformations, and can have a wide range of molecular weight, and polymer subunits. These subunits may include a biological polymer, a synthetic polymer, or a combination thereof.

Many derivatives of such biological water-soluble polymers are known, and are embodied herein. Examples of synthetic water-soluble polymers include, but are not limited to (a) polymers comprising polyalkylene oxide, polyethylene oxide, ethylene/maleic anhydride copolymer and derivatives thereof, including homopolymers and copolymers thereof such as polyethylene glycol, monomethoxy polyethylene glycol, polypropylene glycol homopolymers, copolymers of ethylene glycol with propylene glycol, and derivatives thereof, wherein such homopolymers and copolymers are unsubstituted or substituted at one end with an alkyl group; (b) polyvinyl alcohol and polyvinyl ethyl ethers; (c) polyvinylpyrrolidone; (d) polyoxyethylated polyols, including pluronic polyols; (e) polyesters such as poly(glycolic acid); poly(L-lactic acid); poly(D-lactic acid); poly(DL-lactic acid); lactide/glycolide copolymers; poly-1,3,6-trioxane; poly-1,3-dioxolane; poly(p-dioxanone) and copolymers; polycaprolactone; PEG-polyester copolymers; poly(phosphate esters); (f) poly(orthoesters); (g) polyanhydrides; (h) pseudo-poly(amino acids) (either homopolymers or random copolymers); (i) polyglycerol; (j) dextran, carboxymethylcellulose; and the like.

Water-soluble polymers such as those described above are well known and have been employed with various attachment chemistries, linkage systems and structures as biostable, biodegradable as well as pro-drug constructs for linkage to peptides, polypeptides and other compounds. (See, e.g., International Patent Applications: WO 90/13540, WO 92/00748, WO 92/16555, WO 94/04193, WO 94/14758, WO 94/17039, WO 94/18247, WO 94/28937, WO 95/11924, WO 96/00080, WO 96/23794, WO 98/07713, WO 98/41562, WO 98/48837, WO 99/30727, WO 99/32134, WO 99/33483, WO 99/53951, WO 01/26692, WO 95/13312, WO 96/21469, WO 97/03106, WO 99/45964, and U.S. Pat. Nos. 4,179,337; 5;075,046; 5,089,261; 5,100,992; 5,134,192; 5,166,309; 5,171,264; 5,213,891; 5,219,564; 5,275,838; 5,281,698; 5,298,643; 5,312,808; 5,321,095; 5,324,844; 5,349,001, 5,352,756; 5,405,877; 5,455027; 5,446,090; 5,470,829; 5,478,805; 5,567,422; 5,605,976; 5,612,460; 5,614549; 5,618,528; 5,672,662; 5,637,749; 5,643,575; 5,650,388; 5,681,567; 5,686,110; 5,730,990; 5,739,208; 5,756,593; 5,808,096; 5,824,778; 5,824,784; 5,840,900; 5,874,500; 5,880,131; 5,900,461; 5,902,588; 5,919,442; 5,919,455; 5,932,462; 5,965,119; 5,965,566; 5,985,263; 5,990,237; 6,011,042; 6,013,283; 6,077, 939; 6,113,906; 6,127,355; 6,177,087; 6,180,095; 6,194,580; 6,214,966). Additional information concerning suitable polymers is provided by Turnbull, W. B. et al. (Calbiochem (2000), Toward the Synthesis of Large Oligosaccharide-Based Dendrimers,” 1:70-74), van Duijvenbode, R. C. et al. (Macromolecules (2000) “Synthesis and Protonation Behavior of Carboxylate-Functionalized Poly(propyleneimine) Dendrimers,” 33:46-52), Marcaurelle, L. A. (“Recent Advances in the Synthesis of Mucin-Type Glycoproteins,” http://www.chem.unt.edu/acs/pdf/marcaur.pdf), Domenek, S. (“The Human Genome Project: Challenge for Society and Chemistry,” http://vww.orgc.tugraz.at/orgc/hoegroup/chem ges/domenek.PDF), Imperiali, B. et al. (“Effect of N-linked glycosylation on glycopeptide and glycoprotein structure,” Current Opinion in Chemical Biology 1999, 3:643-649), Rudd, P. M. et al. (“Glycosylation and the Immune System,” Science (2000) 291:2370-2376), Van den Steen, P. et al. (“Concepts and Principles of O-Linked Glycosylation,” Critical Reviews in Biochemistry and Molecular Biology, 33(3):151-208 (1998)), Knischka, R. et al. (“Star-Shaped Polymers Based On Polyglycerol Cores: Synthesis, Characterization And Crosslinking,” http://www-ics.u-strasbg.fr/˜equipe3/Poster4.pdf), Rudd, P. M. et al. (“Roles for Glycosylation of Cell Surface Receptors Involved in Cellular Immune Recognition, J. Mol. Biol. (1999) 293, 351-366), and Davis, B. G. (“Recent developments in glycoconjugates,” J. Chem. Soc., Perkin Trans. 1 1999, (22), 3215-3237).

The water-soluble polymers of the present invention will preferably have an effective hydrodynamic molecular weight of greater than 10,000 daltons (“Da”), and more preferably about 20,000 to 500,000 Da, and most preferably about 40,000 to 300,000 Da. By “effective hydrodynamic molecular weight” is intended the effective water-solvated size of a polymer chain as determined by aqueous-based size exclusion chromatography (SEC). When the water-soluble polymer contains polymer chains having ethylene oxide repeat units, it is preferred that each chain have an atomic molecular weight of between about 200 and about 80,000 Da and preferably between about 1,500 and about 42,000 Da, with 2,000 to about 20,000 Da being most preferred. Unless referred to specifically, molecular weight is intended to refer to atomic molecular weight.

Such water-soluble polymers may include polymer chains that are biostable or biodegradable. For example, polymers with repeat linkages have varying degrees of stability under physiological conditions depending on bond lability. Polymers with such bonds can be categorized by their relative rates of hydrolysis under physiological conditions based on known hydrolysis rates of low molecular weight analogs, e.g., from less stable to more stable polycarbonates (—O—C(O)—O—)>polyesters (—C(O)—O—)>polyurethanes (—NH—C(O)—O—)>polyorthoesters (—O—C((OR)(R′))—O—)>polyamides (—C(O)—NH—). Similarly, the linkage systems attaching a water-soluble polymer to a target molecule may be biostable or biodegradable, e.g., from less stable to more stable carbonate (—O—C(O)—O—)>ester (—C(O)—O—)>urethane (—NH—C(O)—O—)>orthoester (—O—C((OR)(R′))—O—)>amide (—C(O)—NH—). These bonds are provided by way of example, and are not intended to limit the types of bonds employable in the polymer chains or linkage systems of the water-soluble polymers of the invention. As noted above, bioactivity of a synthetic protein of the invention can be modified by altering the nature of the water-soluble polymer that is attached thereto.

A preferred water-soluble polymer for this purpose has a formula:
U-B-Polymer-J
where U is a residue of a functional group that is capable of being attached or is attached to a target molecule, B is a branching core having three or more arms and may be present or absent, Polymer is a substantially non-antigenic water-soluble polymer having a molecular weight equivalent to or greater than 1000 Da, and J is a pendant group having a net charge under physiological conditions selected from negative, neutral or positive. Such polymers include those described in U.S. Patent Application Ser. No. 60/236,377, which is incorporated herein in its entirety.

Components U, B, Polymer and J may be separated from other groups of the water-soluble polymer by one or more spacer or linker moieties. Thus water-soluble polymer U-B-Polymer-J may be represented by the formula:
U-s₁-B-s₂-Polymer-s₃-J
where s₁, S₂, and S₃ are spacer or linker moieties that may be the same or different, and may be individually present or absent. Preferred spacers or linkers include linear or branched moieties comprising one or more repeat units employed in a water-soluble polymer, diamino and or diacid units, natural or unnatural amino acids or derivatives thereof, as well as aliphatic moieties, including alkyl, aryl, heteroalkyl, heteroaryl, alkoxy, and the like, which preferably contain up to 18 carbon atoms or even an additional polymer chain.

As noted above, component U is a residue of a functional group that is capable of being attached or is attached to a target molecule, such as a peptide or polypeptide. When U is a residue of a functional group for conjugation to a target molecule, U comprises a nucleophilic group or electrophilic group, and the target molecule comprises a mutually reactive electrophilic group or nucleophilic group, respectively.

The above-descrived polymer may be used to produce a water-soluble polymer-modified protein comprising the formula: Protein-W, wherein W is the water soluble polymer group of formula: -s0-U-s1-B-s2-Polymer-s3-J; wherein U is a residue of a functional group that is attached to the protein at the protein directly or through s0, and wherein B is a branching core having three or more arms and may be present or absent, Polymer is a substantially non-antigenic, water-soluble polymer having a molecular weight equivalent to or greater than 1000 Da, J is a pendant group having a net charge under physiological conditions selected from negative, neutral or positive, and s0, s1, s2 and s3 are spacer or linker moieties that may be the same or different, and may be individually present or absent.

Many such mutually reactive functional groups are known and are capable of attachment to side chain functional groups common to peptides and polypeptides, or derivatized side chain functional groups (Zalipsky et al., Bioconjugate Chemistry (1995) 6:150-165; “Perspectives in Bioconjugate Chemistry”, C. F. Meares, Ed., ACS, 1993; “Chemistry of Protein Conjugation and Cross-Linking”, S. S. Wong, Ed., CRC Press, Inc. (1993)). Examples of functional groups include groups capable of reacting with an amino group such as (a) carbonates such as the p-nitrophenyl, or succinimidyl; (b) carbonyl imidazole; (c) azlactones; (d) cyclic imide thiones; and (e) isocyanates or isothiocyanates. Examples of functional groups capable of reacting with carboxylic acid groups and reactive carbonyl groups include (a) primary amines; or (b) hydrazine and hydrazide functional groups such as the acyl hydrazides, carbazates, semicarbamates, thiocarbazates, aminooxy etc. Functional groups capable of reacting with mercapto or sulfhydryl groups include phenyl glyoxals, maleimides, and halogens. Examples of functional groups capable of reacting with hydroxyl groups such as (carboxylic) acids, or other nucleophiles capable of reacting with an electrophilic center, include hydroxyl, amino, carboxyl, thiol groups, active methylene and the like.

For instance, water-soluble polymers can be prepared that carry component U as a functional group for attachment to a target molecule, where the functional group is acrylate, aldehyde, ketone, aminooxy, amine, carboxylic acid, ester, thioester, halogen, thiol, cyanoacetate, dipalmitoyl phosphatidylethanolamine, distearoyl phosphatidylethanolamine, epoxide, hydrazide, azide, isocyanate, maleimide, methacrylate, nitrophenyl carbonate, orthopyridyl disulfide, silane, sulfhydryl, vinyl sulfones, succinimidyl glutarate, succimidyl succinate, succinic acid, tresylate and the like. U also may be provided in an activatable form, e.g., a carboxylic acid that can be converted to an active ester thereof that is capable of reacting with a nucleophile such as an amine.

In a preferred embodiment, U is a residue of a unique functional group that is selectively reactive with a unique functional group on the target molecule. This aspect of the invention embodies the principles of peptide synthesis (protecting group strategies) and chemical ligation (partial or no protecting group strategies). For the protecting group strategy, all potentially reactive functional groups except for U and its mutually reactive functional group present on the target molecule are blocked with suitable protecting groups. Many protecting groups are known and suitable for this purpose (See, e.g., “Protecting Groups in Organic Synthesis”, 3rd Edition, T. W. Greene and P. G. M. Wuts, Eds., John Wiley & Sons, Inc., 1999; NovaBiochem Catalog 2000; “Synthetic Peptides, A User's Guide,” G. A. Grant, Ed., W. H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; and “Protecting Groups,” P. J. Kocienski, Ed., Georg Thieme Verlag, Stuttgart, Germany, 1994).

Thus, U can represent a residue of a wide range of functional groups, such as those described above. For the partial or no protecting group strategy, U and its mutually reactive functional group present on the target molecule employ a chemoselective reaction pair in which other functional groups may be present in the reaction system but are unreactive. This includes U groups amenable to amine capture strategies (e.g., ligation by hemiaminal formation, by imine formation, and by Michael addition), thiol capture strategies (e.g., ligation by mercaptide formation, by disulfide exchange), native chemical ligation strategies (e.g., ligation by thioester exchange involving cysteine or thiol contain side-chain amino acid derivative), and orthogonal ligation coupling strategies (e.g., ligation by thiazolidine formation, by thioester exchange, by thioester formation, by disulfide exchange, and by amide formation)(See, e.g., Coltart, D M., Tetrahedron (2000) 56:3449-3491).

A preferred U comprises a residue of a unique functional group employed in an aqueous compatible ligation chemistry such as native chemical ligation (Dawson, et al., Science (1994) 266:776-779; Kent, et al., WO 96/34878), extended general chemical ligation (Kent, et al., WO 98/28434), oxime-forming chemical ligation (Rose, et al., J. Amer. Chem. Soc. (1994) 116:30-33), thioester forming ligation (Schnolzer, et al., Science (1992) 256:221-225), thioether forming ligation (Englebretsen, et al., Tet. Letts. (1995) 36(48):8871-8874), hydrazone forming ligation (Gaertner, et al., Bioconj. Chem. (1994) 5(4):333-338), and thiazolidine forming ligation and oxazolidine forming ligation (Zhang, et al., Proc. Natl. Acad. Sci. (1998) 95(16):9184-9189; Tam, et al., WO 95/00846) or by other methods (Yan, L. Z. and Dawson, P. E., “Synthesis of Peptides and Proteins without Cysteine Residues by Native Chemical Ligation Combined with Desulfurization,” J. Am. Chem. Soc. 2001, 123, 526-533, herein incorporated by reference; Gieselnan et al., Org. Lett. 2001 3(9):1331-1334; Saxon, E. et al., “Traceless” Staudinger Ligation for the Chemoselective Synthesis of Amide Bonds. Org. Lett. 2000, 2, 2141-2143).

Given the various attachment chemistries described above, it will be apparent that when U is a residue of a functional group conjugated to a target molecule, U can comprise a residue of a bond selected from carbonate, ester, urethane, orthoester, amide, amine, oxime, imide, urea, thiourea, thioether, thiourethane, thioester, ether, thaizolidine, hydrazone, oxazolidine and the like. Preferred bonds are oxime and amide bonds.

As noted above, B is a branching core moiety having three or more arms, and may be present or absent. When B is present, one arm is joined to U or a spacer or linker attached to U, and each other arm is joined to a Polymer or a spacer or linker attached to a Polymer. Examples of branching cores B include, but are not limited to, amino, amide, carboxylic, and combinations thereof. These include oligoamides of lysine and/or arginine or a combination thereof, or oligomers prepared from alkanediamines and acrylic acid (“polyamidoamines”), the later providing a net positive charge in the branching core. (See, e.g., Zeng et al., J. Pept. Sci. (1996) 2:66-72; Rose et al., Bioconjugate Chem., (1996) 7(5):552-556; NovoBiochem Catalog and Peptide Synthesis Handbook, Laufelfingen, 2001; Wells et al., Biopolymers (1998) 47:381-396; Mahajan et al., (1999) 40:4909-49-12; Judson et al. (1998) 39:1299-1302; Swali et al., (1997) 62:4902-4903; U.S. Pat. Nos. 5,932,462, 5,919,455, 5,643,575, 5,681,567). Many other different branching cores can be used and are suitable for this purpose, including substituted diamines, substituted diacids, alkyl acids such as glycerol and other moieties having three or more functional or activatable groups including multivalent alkyl, aryl, heteroalkyl, heteroaryl, and alkoxy moities and the like, and oligosaccharides (e.g., Nierengarten et al., Tetrahedron Lett. (1999) 40:5681-5684; Matthews et al., Prog. Polym. Sci. (1998) 1-56; Suner et al., Macromolecules (2000) 33:253; Fischer et al., Angew. Chem. Int. Ed. (1999) 38:884; Sunder et al., Adv. Mater. (2000) 12:235; Mulders et al., Tetrahedron Lett. (1997) 38:631-634; Mulders et al., Tetrahedron Lett. (1997) 38:30-3088; and Turnbull et al., Chembiocehm (2000) 1(1):70-74).

Preferred branching cores are amino, carboxylic and mixed amino and carboxylic. Moreover, preferred branched polymer constructs are those in which branching emanates from a single branching core, such as an oligoamide or polyamidoamine core. However, other classes of branched constructs can be employed, such as worm-like structures in which the branching emanates from a sequence of multiple branching cores distributed along a polymer backbone (Schliiter et al., Chem. Int. Ed. (2000) 39:864). Depending on the chemical composition and/or the bulkiness of polymer backbone and repeat units, the resulting worm-like structures can be designed to be water soluble or prone to induce liquid crystalline organization (Ouali et al., Macromolecules (2000) 33:6185), which can be advantageous for delivery applications, stability and duration of action. As with the other branched polymer constructs of the invention, the worm-like constructs are made to contain a pendant functional group comprising U.

The component Polymer of the water-soluble polymer of the formula U-B-Polymer-J includes those water-soluble polymers described above. A preferred Polymer component is selected from (a) polymers comprising polyalkylene oxide, polyethylene oxide, and derivatives thereof, including homopolymers and copolymers thereof such as polyethylene glycol, monomethoxy polyethylene glycol, polypropylene glycol homopolymers, copolymers of ethylene glycol with propylene glycol, and derivatives thereof, wherein such homopolymers and copolymers are unsubstituted or substituted at one end with an alkyl group; (b) polyvinyl alcohol and polyvinyl ethyl ethers; (c) polyvinylpyrrolidone; (d) polyoxyethylated polyols, including pluronic polyols; (e) polyesters such as poly(glycolic acid); poly(L-lactic acid); poly(D-lactic acid); poly(DL-lactic acid); lactide/glycolide copolymers; poly(p-dioxanone) and copolymers; polycaprolactone; PEG-polyester copolymers; poly(phosphate esters); (f) poly(orthoesters); (g) polyanhydrides; (h) pseudo-poly(amino acids); and (i) polyglycerol; and the like. Among these the preferred Polymer comprise polyalkylene oxide, particularly those comprising polyethylene oxide of the formula (—CH₂—CH₂—O—)ₙ or (O—CH₂—CH₂—)ₙ where n is 2 or more, and derivatives thereof, including homopolymers and copolymers thereof. (See.e.g., “Poly(ethylene glycol) Chemistry: Biotechnical and Biomedical Applications”, J. M. Harris, Ed., Plenum Press, New York, N.Y. (1992); and “Poly(ethylene glycol) Chemistry and Biological Applications”, J. M. Harris and S. Zalipsky, Eds., ACS (1997).

The more preferred Polymer component is where the water-soluble polymer U-B-Polymer-J is produced in total by stepwise synthesis. This means that these polymers will have a precise molecular weight and defined structure. In contrast, normal polymer synthesis, which is a polymerization process, results in a mixture in which chains are of differing lengths, and so there is a distribution of molecular weights and sizes that are difficult if not impossible to separate. The ability to control molecular purity is advantageous in that a synthetic protein can be constructed that has a water-soluble polymer attached thereto and that is monodisperse. This represents a significant advantage in that variable properties resulting from heterogeneous compounds can be avoided, and only those compounds with the most preferred properties can be prepared and isolated with relative ease.

The most preferred Polymer component comprise polyamides of the formula —[C(O)—X—C(O)—NH—Y—NH]n— or —[NH—Y—NH—C(O)—X—C(O)]n— as described in WO 00/12587, where n is a positive integer from 1-100 and more preferably from 2-100, and where X and Y are biocompatible repeat elements of precise structure linked, for example, by an amide bond. X and Y may be divalent radicals, etc. X and Y may be the same or different and may be branched or linear. In highly preferred examples, at least one of X and Y comprises a water-soluble polymer repeat unit.

A preferred water-soluble repeat unit for X, Y comprises a polyalkylene oxide, a polyethylene oxide, and derivatives thereof, including homopolymers and copolymers thereof, wherein such homopolymers and copolymers can be unsubstituted or substituted, linear or branched, and may include flanking groups comprising alkyl, aryl, arylalkyl groups and the like, for example polyoxyethylated polyols, including pluronic polyols, and C1 to C18 aliphatic flanking groups. It will be appreciated that minor variations include polyamides of the formula —[C(O)—X—NHC(O)—Y—NH]n— or —[NH—Y—C(O)NH—X—C(O)]n. X′ and Y′ are sub-embodiments of X and Y where X═X′—NH or NH—X′ and Y═Y′—NH or NH—Y′. A more preferred water-soluble repeat unit for X′, Y′ comprises a polyethylene oxide formula (—CH₂—CH₂—O—)n or (O—CH₂—CH₂—)n where n is 2 to 50, 3 to 25, 3 to 10, and more preferably 3 to 5.

As noted above, component J can be any group having a net charge under physiological conditions selected from neutral, positive or negative. This includes alkyl, aryl, arylalkyl, acyl, and carbonyl groups, that are substituted or unsubstituted, and as well as salts thereof. Such groups may be a component of amino acids, nucleic acids, fatty acids, carbohydrates, and derivatives thereof, and moieties such as chitin, chitosan, heparin, heparan sulfate, chondroitin, chondroitin sulfate, dermatan and dermatan sulfate, cyclodextrin, dextran, hyaluronic acid, phospholipid, sialic acid and the like. J preferably comprises an ionizable moiety selected from carboxyl, amino, thiol, hydroxyl, phosphoryl, guanidinium, imidazole and salts thereof. The most preferred is where J comprises an ionizable carboxylate moiety and has a net negative charge under physiological conditions.

In accordance with the present invention, a preferred solution to the problems of polymer heterogeneity, diversity, and unsuitability involves the production of a new class of biocompatible polymers which combine the advantages of both polypeptides (precise length, convenient synthesis) and glycosylation-mimicking groups (“glyco-mimetic groups”), a flexible, amphiphilic, non-immunogenic, polymer not susceptible to proteases) Rose, K. et al. (U.S. patent application Ser. No. 09/379,297, herein incorporated by reference).

In a preferred embodiment of such preferred Polymers, —[C(O)—X—NHC(O)—Y—NH]n— or —[NH—Y—C(O)NH—X—C(O)]n, X and Y will be divalent organic radicals lacking reactive functional groups or will be absent and may be the same or different, and can vary independently with each repeating unit (n). Preferably, when n=2, at least one of X or Y will be selected from the group consisting of a substituted, unsubstituted, branched and linear aliphatic and aromatic group. More preferably, the divalent organic radicals will be selected from the group consisting of phenyl, a C₁-C₁₀ alkylene moiety, a C₁-C₁₀ alkyl group, a heteroatom-containing phenyl, a heteroatom-containing C₁-C₁₀ akylene moiety, a heteroatom-containing C₁-C₁₀ alkyl group, and a combination thereof. Particularly preferred glyco-mimetic moieties can be represented by the formula:
—{CO—(CH₂)₂—CO—NH—(CH₂)₃—OCH₂CH₂)₃—CH₂—NH}ₙ—
where n preferably varies from 1-100 and more preferably from 2-100; or
—{CO—(CH₂)₂—CO—NH—(CH₂)₆—NH—CO—(CH₂)₂—CO—NH—(CH₂)₃—(OCH₂CH₂)₃—CH₂—NH—}ₙ—,
where n preferably varies from 1-50 and more preferably from 2-50. A particularly preferred glyco-mimetic group has n=12, X is —(CH₂)₂—, and Y is:
—(CH₂)₃O(CH₂)₂O(CH₂)₂O(CH₂)₃—.

The glyco-mimetic moieties of the present invention can be synthesized in any of a variety of ways. Such moieties are, however, preferably produced using a solid phase stepwise chain assembly of units, rather than a polymerization process. The use of such an assembly process permits the moieties of a preparation to have a defined and homogeneous structure, as to their length, the nature of their X and Y substituents, the position(s) (if any) of branch points, and the length, X and Y substituents, and position(s) of any branches. Preferably, such moieties will be synthesized by steps such as:
(a) acylating the amino or hydroxyl group of a compound of the formula Z-Q-support with a molar excess of a derivative of a diacid having the formula, HOOC—X—COOH, where Z is H₂N— or HO—; Q is a linker or a target molecule; and the support is a solid phase, matrix or surface;
(b) activating the free carboxyl group of the product of step (a);
(c) aminolysing the product of step (b) with a molar excess of a diamine having the formula, NH₂—Y—NH₂; and
(d) optionally repeating steps (a)-(c) using HOOC—X—COOH and NH₂—Y—NH₂, where said X and Y are divalent organic radicals or are absent and are the same or different, and can vary independently with each of said optionally repeated units, and are the same or different from the X and Y substituents used in any of the previous acylating and aminolysing steps.

In preferred embodiments, 6-mers, 12-mers, 18-mers and 32-mers of above repeat unit are employed. Where desired, the repeat unit can be used (for example, in conjunction with the amino group of lysine to form branched glyco-mimetic structures. The glyco-mimetic groups may be attached to the synthetic proteins of the present invention by a varietiy of chemistries, including thioether, oxime and amide linkage formation.

In one embodiment, the solid phase stepwise chain assembly of units comprises:
Step 1: Couple protected or unprotected diacid to amino on resin to generate amide bond at linkage site (where PG is a protecting group that is present or absent depending on diacid employed):
   PG—OOC—X—COOH+NH₂—Y—NH-Resin
Step 2: Remove protecting group (PG) on resin, if present
   HOOC—X—CO—NH—Y—NH-Resin
Step 3: Couple protected or unprotected diamino to carboxy on resin to generate amide bond at linkage site (where PG is present or absent depending on diamino employed)
   PG—NH—Y—NH+HOOC—X—CO—NH—Y—NH-Resin
Step 4: Remove protecting group (PG) on resin, if present
   —NH—Y—NH—OC—X—CO—NH—Y—NH-Resin
Step 5: Repeat steps 1-4 ‘n’ times to add ‘n’ units then cleave from resin
   —[CO—X—CO—NH—Y—NH]—[CO—X—CO—NH—Y—NH]—[CO—X—CO—NH—Y—NH]—[CO—X—CO—NH—Y—NH]

3. The Synthesis of the Water-Soluble Polymers Of The Present Invention

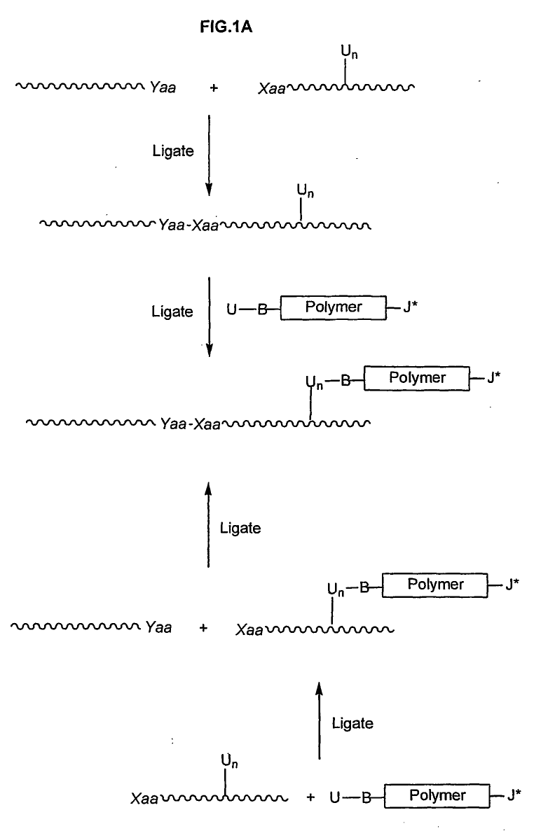

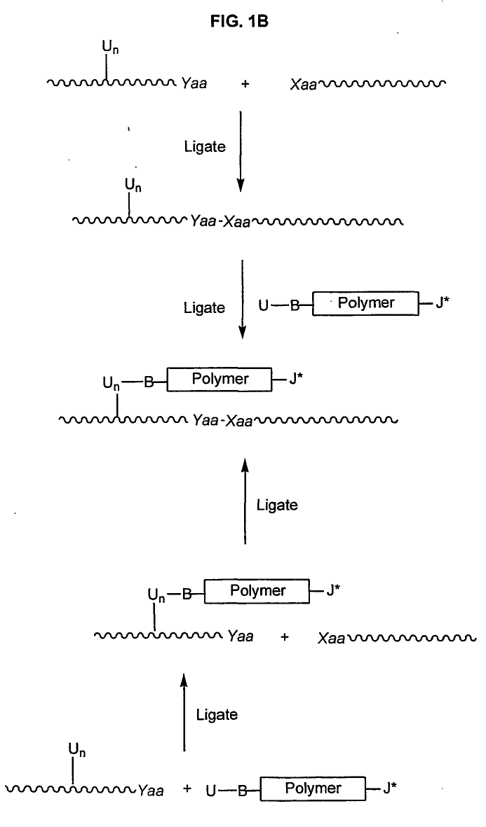

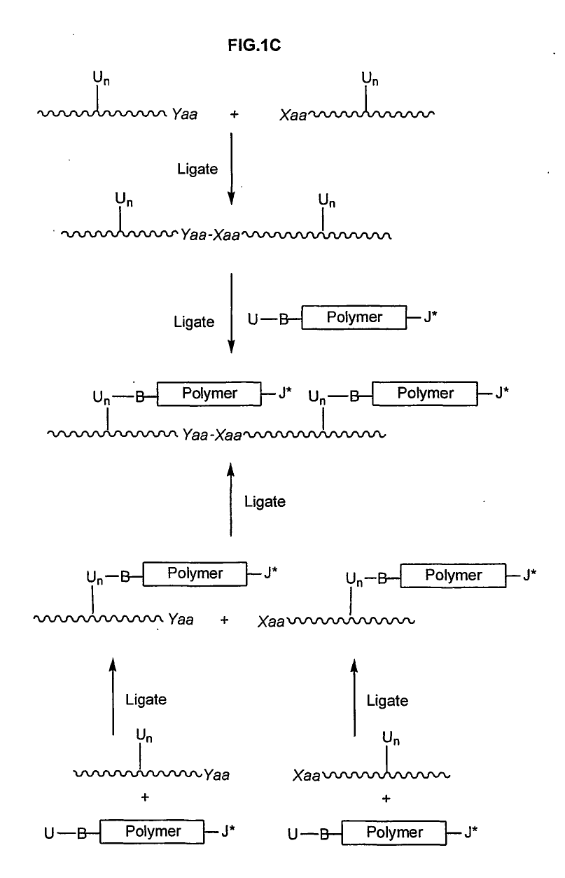

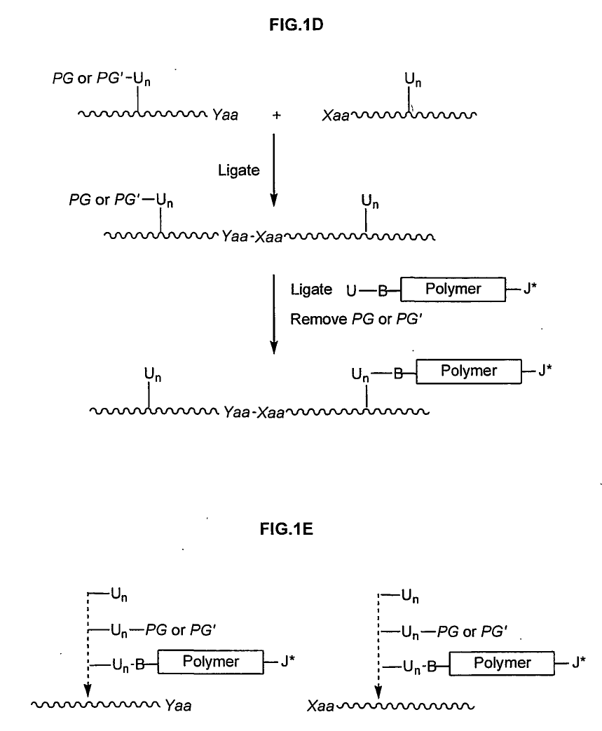

The water-soluble polymers of the present invention can be synthesized by any of variety of methods. FIGS. 1A-1E depict ligation schemes involving the attachment of a water-soluble polymer (U-B-Polymer-J* as defined herein) to partially or fully unprotected peptide segments [symbol] before or after ligation, or combinations thereof. In FIGS. 1A-1D, Yₐₐ represents the C-terminal amino acid on a first peptide segment that bears a unique chemoselective moiety (e.g., amino acid bearing alpha-carboxyl thioester) for chemical ligation to a second peptide segment bearing a unique and mutually reactive N-terminal amino acid Xₐₐ (e.g., amino terminal cysteine) moiety that is capable of chemoselective chemical ligation with Yₐₐ. Chemoselective reaction between Yₐₐ and Xₐₐ generate a covalent linkage therein between (e.g., amide bond). Thus Yₐₐ and Xₐₐ form a chemoselective ligation pairing. Uₙ-, as shown in the Figures, represents a second unique chemoselective moiety that has been incorporated at a precise user-defined site on the side chain of an amino acid and is chemoselective for, and mutually reactive with group U- of the water-soluble polymer U-B-Polymer-J*. For example, when Un is a side chain that has been modified to bear a ketone group, U- of the water-soluble polymer U-Polymer-J* is a group chemoselective for reacting with the ketone, e.g., an aminooxy group which yields an oxime bond therein between. The subscript “n” of Uₙ- represent the number of amino acids and their side chains designed to bear chemoselective group U, for example, where two specific and user-defined sites are to be polymer modified n=2, which also can be represented as U₂ or U₌₂. In all cases, n is a positive integer that is precisely controlled by design. Thus Un and U represent a second chemoselective ligation pairing that is compatible and unreactive with chemoselective groups of Xaa and Yaa. FIGS. 1A and 1B illustrate two different potential reactions. In the first depicted reaction, a polypeptide chain beairing a Un functionality is ligated to a second polypeptide, and is then reacted with a U-B-Polymer-J* moiety in order to obtain a polymer-modified polypeptide. In the second depicted reaction, the polypeptide chain beairing the Uₙ functionality is reacted with a U-B-Polymer-J* moiety in order to obtain a polymer-modified polypeptide, and then ligated to a second polypeptide, to obtain a larger polymer-modified polypeptide. The figures differ in that in FIG. 1A, the polypeptide bearing the Xaa residue is to receive the polymer modification, whereas in FIG. 1B, the polypeptide bearing the Yaa residue receives the polymer modification. FIG. 1C illustrates the ability of the present invention to modify multiple poly eptide chains, either before or after their ligation to form a larger polypeptide. In FIG. 1D, PG and PG′ represent protecting groups, where PG′ depicts an orthogonal protecting group, i.e., PG and PG′ are removable under different conditions, and are useful where different water-soluble polymers are attached via same chemistry to Un groups, or where Un groups represent side-chain functional groups that one does not wish to modify with a polymer (e.g., side chains bearing reactive —NH₂ or —SH where U group of water-soluble polymer is designed to react exclusively with primary amino or side chain thiols). FIG. 1D shows that protecting groups can be employed in order to protect desired side chains of the polypeptides being ligated in accordance with the methods of the present invention.

FIG. 1E illustrates the diversity of groups that may be present or absent in the peptide segments employed in ligation and polymer modification according to FIGS. 1A-1D.

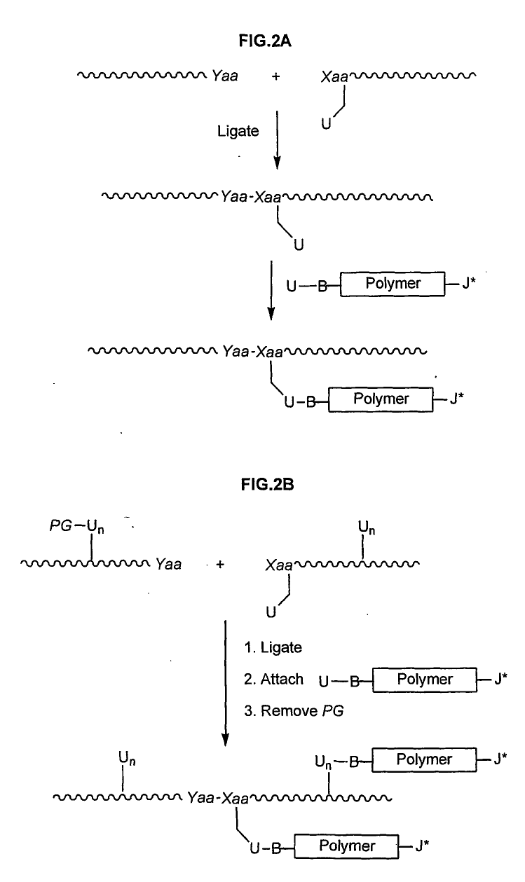

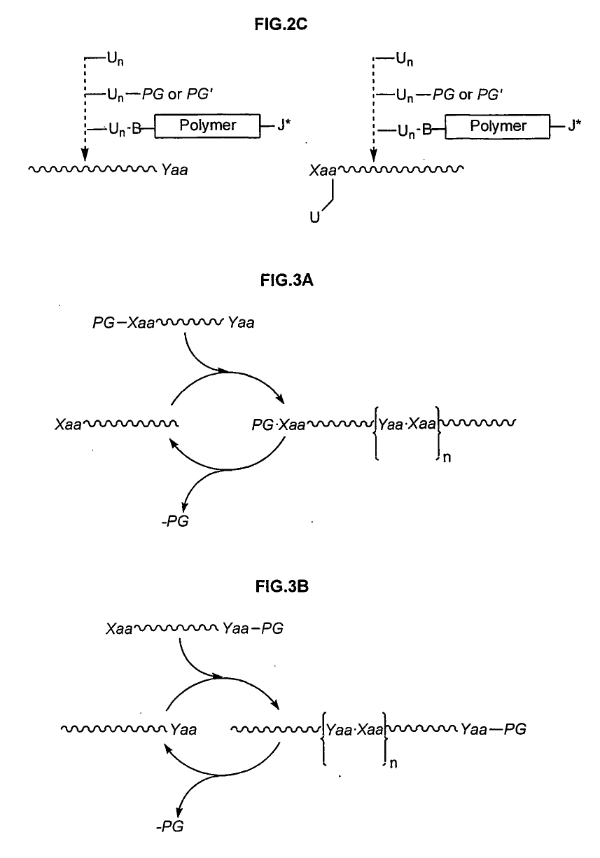

FIGS. 2A-2C depict additional schematics of processes for preparing synthetic bioactive proteins of the invention. In particular, FIGS. 2A-2B depict ligation scheme involving the attachment of a water-soluble polymer U-B-Polymer-J* to the side chain of amino terminal group Xaa at a ligation site (e.g., side chain thiol of cysteine). FIG. 2C illustrates the diversity of groups that may be present or absent in the peptide segments employed in ligation and polymer modification according to FIGS. 2A-2B.

FIGS. 3A-3B depictadditional schematics of processes for accomplishing multi-segment ligations that involve the chemical ligation of three or more non-overlapping peptide segments, i.e., at least one segment is a middle segment corresponding to the final full-length ligation product. Peptides prepared in this manner can be used for preparing peptide segments involved in another ligation reaction, for example, as shown in FIGS. 1A-1E, FIGS. 2A-2C, and FIGS. 4A-4C. In general, for multi-segment ligations, the middle segment(s) has either a protected Xaa group or a protected Yaa group to avoid cyclization or concatomer formation of that peptide depending on the ligation chemistry employed. For sequential or serial ligations, the Xaa group of a middle segment is protected (e.g., Cys(Acm)) while the Yaa group is unprotected (e.g., Yaa-COSR, where —COSR is an alpha-carboxylthioester). Here the Yaa group is free to react with a second peptide bearing an unprotected Xaa group, where the second peptide is devoid of a free Yaa group. Following ligation, the protecting group is removed to regenerate the Xaa group for the next ligation reaction. This process can be continued, as needed thereby generating an elongated polypeptide chain. Protection of the Yaa group is particularly useful for convergent chemical ligation involving the production of a final ligation product composed of four or more segments. For example, for a protein target generated from a four-segment ligation (i.e., three ligation reactions), two segments corresponding to one end of the protein and two segments corresponding to the other end of the protein can be ligated in parallel, as opposed to sequentially, and the two ends joined in a final ligation reaction. Such a convergent chemical ligation scheme is described for thioester-mediated chemical ligation reactions (e.g., native and extended native chemical ligation) in U.S. Patent Application Ser. No. 60/231,339, which is incorporated by reference. Here again the diversity of groups that may be present or absent in such peptide segments are illustrated in FIGS. 1E, 2C and 4C.

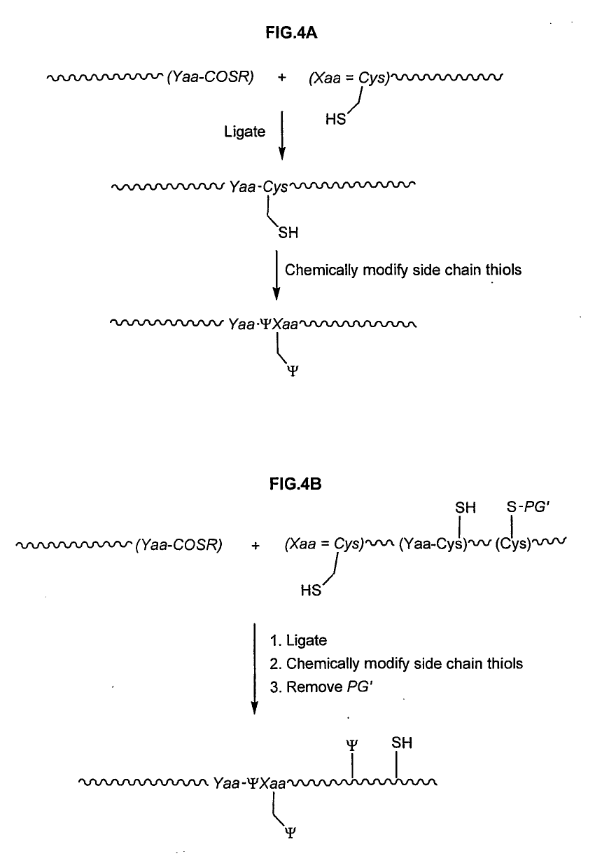

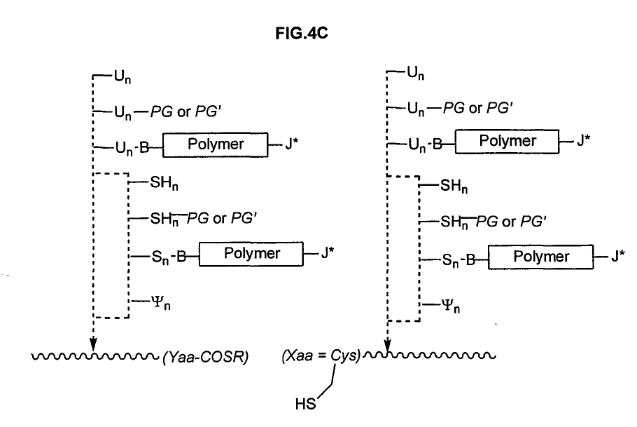

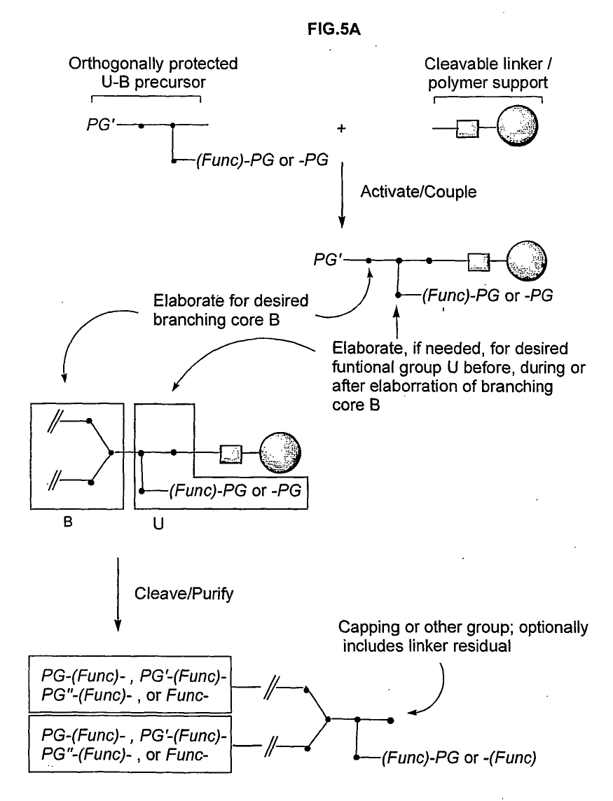

FIGS. 4A-4C illustrate how native chemical ligation and chemical modification of the resulting side-chain thiol may be accomplished in accordance with the principles of the present invention. In particular, FIGS. 4A-4B depict the use of native chemical ligation and chemical modification of the resulting cysteine side-chain thiol at the ligation site(s) to form a “pseudo amino acid” (depicted by ψXaa) via thioalkylation and generation of chemically modified side chain (depicted by ψ) comprising a thioether bond. In an alternative embodiment not depicted, the side chain thiol can be converted to an alanine in a desulfurization reaction (Liang et al, J. Amer. Chem. Soc. (2001) 123(4):526-533). An significant aspect for both reactions is that any other side-chain thiols that one does not wish to convert or modify be protected with a suitable protecting group (PG′) or for multi-segment ligations and orthogonal protecting group (PG′) where the segment bearing the carboxy terminal Yaa group comprises a protected amino terminal Xaa group, i.e., PG-Xaa-peptide, which is provided by way of example in FIG. 4B. FIG. 4C illustrates the diversity of groups that may be present or absent in the peptide segments employed in ligation and polymer modification according to FIGS. 4A-4B, as well as FIGS. 1A-1E, FIGS. 2A-2C, and FIGS. 3A-3B.

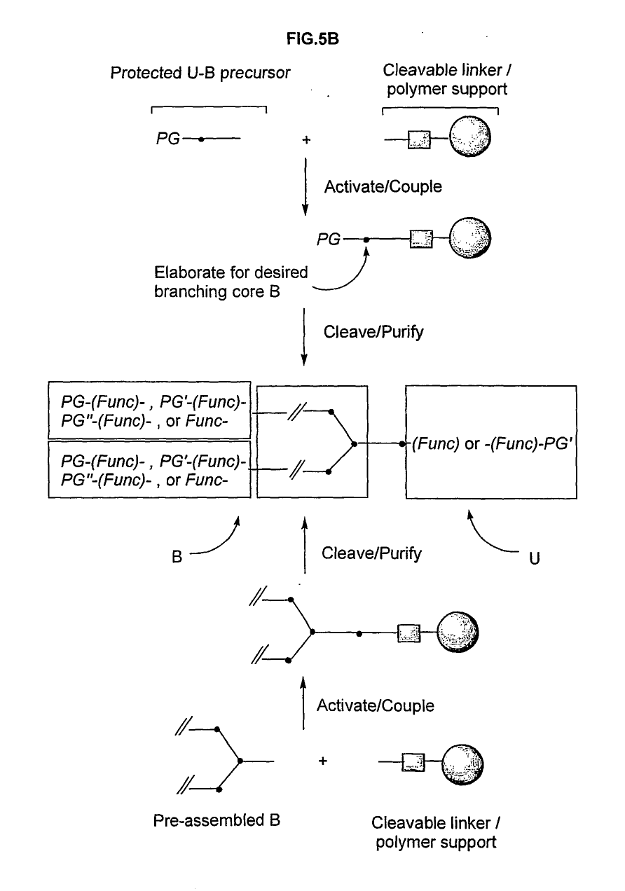

FIGS. 5A-5B depict solid phase process for generating the branching core (B) and unique chemoselective functional group (U) of the water-soluble polymer U-B-Polymer-J* of the invention. The process may be carried out in solution, although the solid phase approach as shown is preferred. In particular, FIG. 5A shows orthogonally protected U-B precursor moiety with reactive group 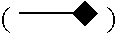
and the basic geometric structure of such construct is depicted by dots linked with bonds 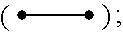
this basic geometric structure is not intended to limit that types of chemical linkages or groups employed, but merely illustrative of the relative points of geometry for building structures and chemical elaboration points suitable for generation of U-B moieties of the invention. The orthogonally protected U-B precursor is coupled to a polymer support/resin comprising a suitable cleavable linker and co-reactive group 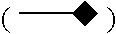
following activation that is capable of covalent linkage to the U-B precursor:

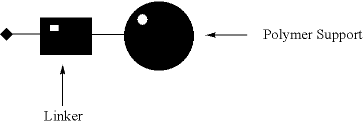

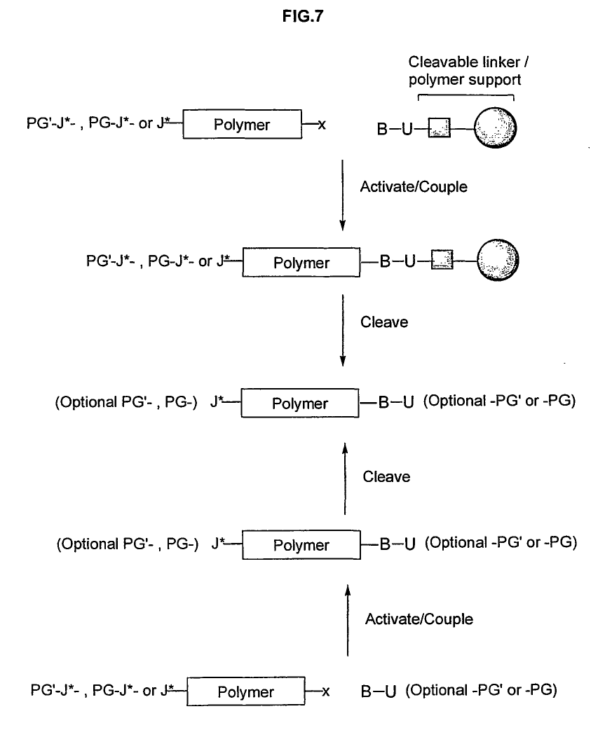

This system employs the principles of polymer-supported organic chemistry. Following coupling the branching core is elaborated (only a first branch point shown, and additional branch points may be present or absent) as illustrated to generate a branching core that is suitable for subsequent attachment of a desired Polymer component, such as a substantially non-antigenic, water-soluble linear polymer. Also shown is the U group, which can be provided at the outset of synthesis as part of the orthogonally protected U-B precursor, or elaborated during or after elaboration of the branching core B. While attachment of the Polymer component can be achieved on-resin, i.e., prior to cleavage, a preferred route is cleavage of the U-B moiety from the polymer support/resin so as to generate a U-B core that can be purified for subsequent attachment the Polymer component. As illustrated, the pendant branch points of core group B are built to comprise a functional group (Func), which can be the same or different, and may be reversibly protected (PG, PG′ or PG′) or unprotected. In each case, the final step for attachment of the Polymer component involves the generation of a functional group (Func) at the pendant branch points, and generation of group U (See FIG. 7). FIG. 5B depicts an alternative process in which a protected U-B precursor is employed in combination with a polymer support bearing a linker, which upon cleavage generates the desired protected or unprotected U-group. FIG. 5B also depicts attachment of a pre-assembled branching core B to a polymer support, and use of a U-group generating resin to make the U-B moiety for subsequent attachment of the Polymer component.

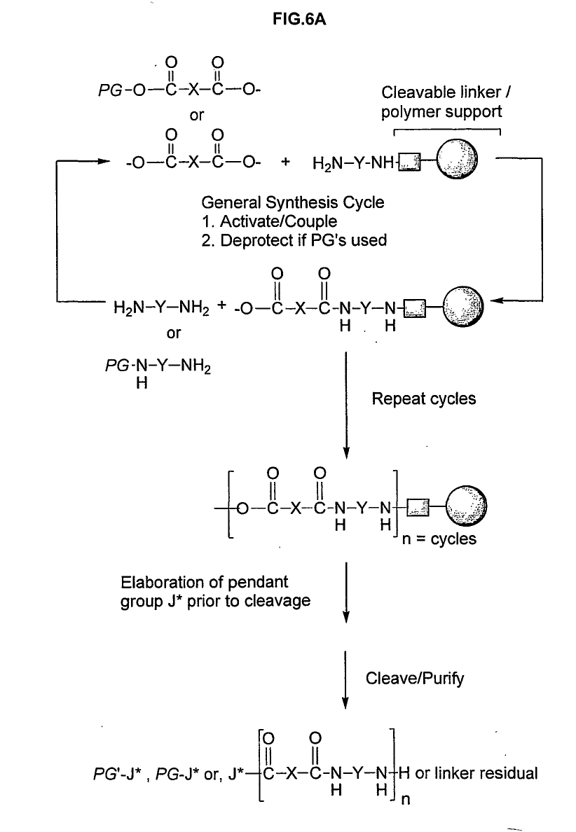

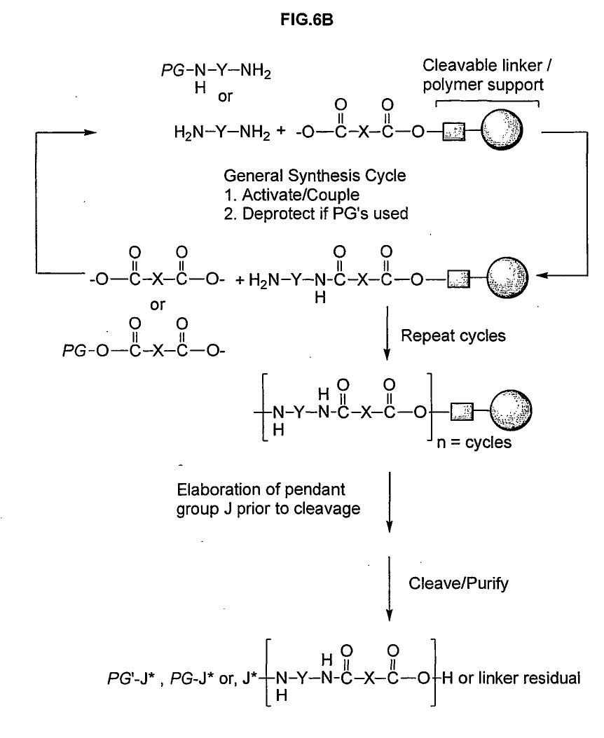

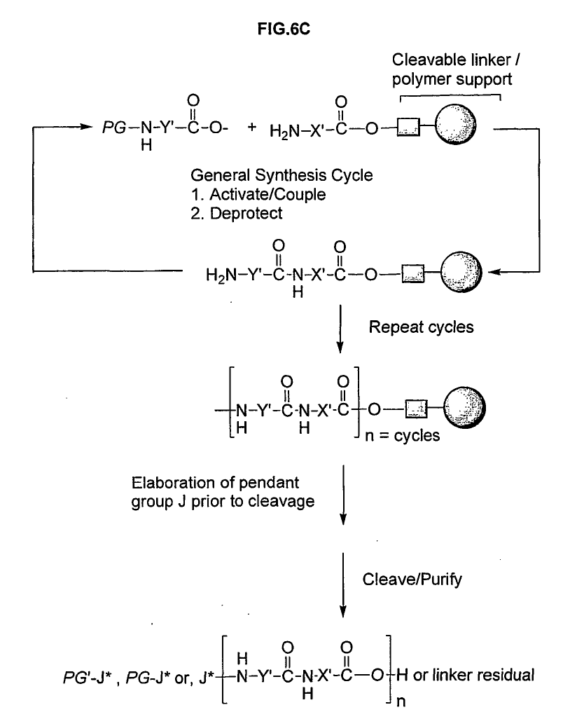

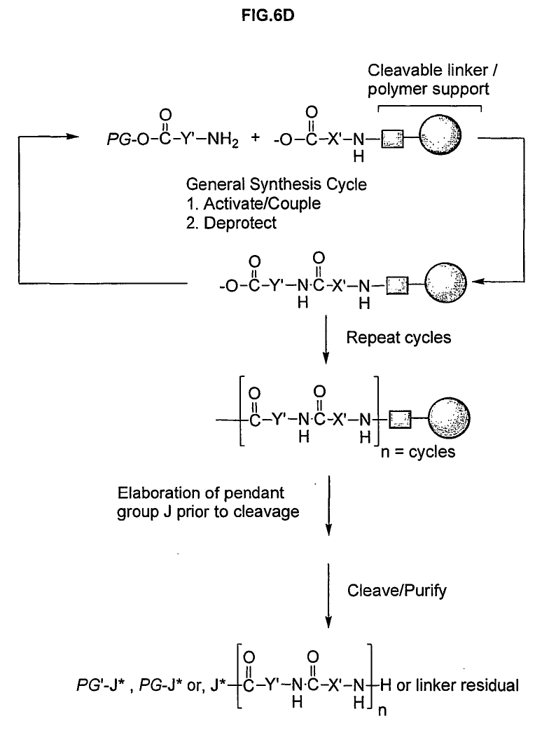

FIGS. 6A-6D depict a solid phase process for generating preferred substantially non-antigenic water-soluble polyamide PolymerJ* components of the invention for subsequent attachment to the U-B core. Although the solid phase process is illustrated, which is the preferred process, a solution phase process can be adapted to achieve the same end result. In FIGS. 6A and 6B, diacid and diamino units are coupled using the principles of solid phase organic chemistry. Optionally, protected amino-X′-acid and/or amino-Y′-acid units can be incorporated for additional diversity of the groups X and Y in the final cleavage product having a polyamide structure of the formula —[NH—Y—NHCO—X—CO]—. FIG. 6A depicts synthesis in the N— to C-terminal direction, whereas FIG. 6B depicts synthesis in the C— to N-terminal direction. In FIGS. 6C and 6D, protected amino-X′-acid and/or amino-Y′-acid units are coupled using the principles of solid phase organic chemistry. FIG. 6C depicts synthesis in the N— to C-terminal direction, whereas FIG. 6D depicts synthesis in the C— to N-terminal direction. As apparent from FIGS. 6A-6D, the nature of the final polyamide products can be precisely controlled, which depends on the number of cycles one carries out for synthesis. Moreover, the pendant group J* can be built to virtually any user-defined specification. Where mono-disperse repeat units X, Y, X′ and Y′ are employed, the exact molecular structure of the final polyamide product can be precisely controlled.

A preferred process for coupling the U-B component to Polymer-J* component to generate the preferred synthetic polymer constructs of the invention of the formula U-B-Polymer-J* is depicted in FIG. 7. As illustrated, various protecting groups can be provided, and are optional depending on the intended end use of a given construct. Also illustrated different routes to produce the desired U-B-Polymer-J* constructs, including a solid phase approach and a solution phase approach.

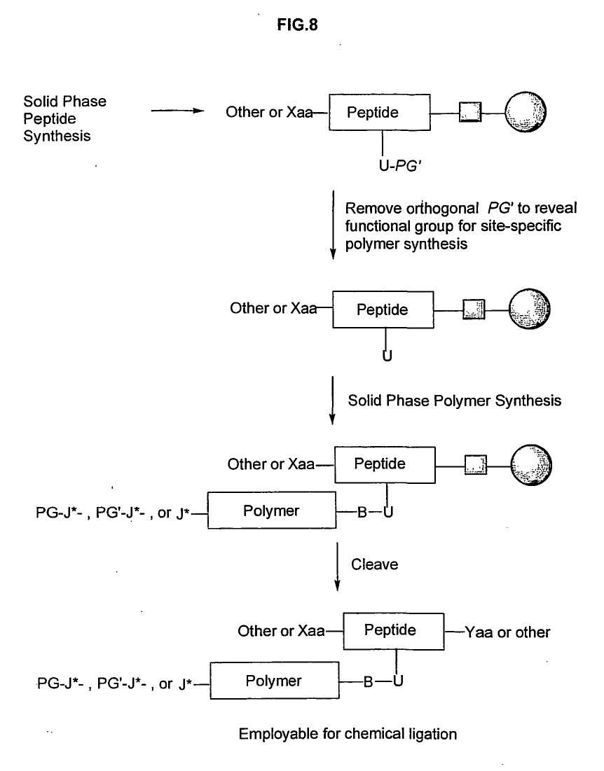

FIG. 8 depicts an alternative route for precision attachment of a water-soluble polymer to a peptide segment employable for ligation and production of bioactive synthetic proteins of the invention. The first step employs solid phase peptide synthesis (“SPPS”) (e.g., Fmoc or Boc SPPS), in which an amino acid side chain targeted for polymer attachment is protected with an orthogonal protecting group (e.g., if using Fmoc SPPS, a Boc group can be used to protect the site of polymer attachment, or if using Boc SPPS, an Fmoc group can be employed as the orthogonal protecting group). Following peptide synthesis, the orthogonal protecting group is selectively removed while the rest of the peptide remains protected. This affords a single attachment site for the next step—solid phase polymer synthesis. Once the orthogonal protecting group is removed, the polymer chain is attached as a precursor. More preferably, the polymer chain is built through successive rounds of polymer synthesis using a process depicted in FIGS. 6A-6D. Although a single polymer attachment site is shown, more than one can be provided.

4. Precursor Chemokine

The third component relates to the generation of polymer-modified bioactive synthetic chemokines having optimal in vitro bioactivity characterized by downregulation of a chemokine receptor to which it binds. This involves selection of a precursor chemokine (i.e., a target chemokine chosen for attachment of a water-soluble polymer thereto) having a potency that is about 1- to 5-fold, and preferably about 5- to 10-fold greater or more (where potency is measured by EC50 in a relevant in vitro cell-based assay) than the desired potency range of the polymer-modified bioactive synthetic chemokine.

By way of example, in identifying a synthetic chemokine comprising a polymer-modified Rantes analog having a desired ED50 for inhibition of viral infection, a panel of non-polymer modified precursor analogs were synthesized and screened in cell-fusion assays for HIV infection. The target anti-fusion potency of the polymer-modified form was set in the range of 10 nM or lower compared to wild type Rantes, which is about 20 nM or higher. In generating a desired precursor Rantes chemokine, a series of modifications were made to (1) the N-terminal residue; (2) internal to the N-terminal region; and (3) to the C-terminal region. A particularly potent precursor analog was found in which the serine corresponding to position 1 of wild type Rantes was by replaced with an n-nonanoyl moiety, the tyrosine corresponding to position 3 of wild type Rantes was replaced with L-cyclohexyl glycine, and a fatty acid moiety was attached to the C-terminal residue through a dipeptide (Lys69-Leu70) linker moiety; an even more potent analog was found which includes these changes in combination with replacing the proline corresponding to position 2 of wild type Rantes with L-thioproline. It was found that when attaching a linear or branched water-soluble polymer at a C-terminal site of such Rantes analogs containing one or more of these changes that the desired target anti-fusion potency of the polymer-modified form was achievable. Namely, a series of synthetic chemokines comprising analogs of Rantes were obtained that had ED50's for inhibition of viral infection in the range of 10 nM or lower. In particular, both linear and branched water-soluble polymer constructs were attached to a C-terminal site (position 67, corresponding to the methionine at position 67 of wild type Rantes) that is adjacent to a known aggregation site in wild type Rantes (position 66, corresponding to the glutamic acid at position 66 of wild type Rantes). The addition of the water-soluble polymer at that site not only disrupted unwanted aggregation, the polymer-modified forms had potency ranges in the target range of 10 nM and lower. This since observation is consistent with the finding that a precursor chemokine targeted for polymer-modification will preferably have a starting potency that is about 1- to 5-fold, and preferably about 5- to 10-fold greater or more (where potency is measured by EC50 in a relevant in vitro cell-based assay) than the desired potency range of the polymer-modified bioactive synthetic chemokine. Moreover, it was found that each of the polymer-modified analogs of Rantes had significantly improved circulating half-life as measured in a relevant animal model, e.g., rat or mouse. This systematic approach of incorporating changes that increase potency of a precursor chemokine is generally applicable to other chemokines. Accordingly, selection of such precursor chemokines can be exploited to yield polymer-modified bioactive synthetic chemokines that exhibit a preferred balance of potency and in vivo serum circulating half-life.

C. Detectable Labeling of the Chemokines of the Present Invention

The bioactive synthetic chemokines of the present invention also may include a detectable label, such as a fluorophore, and other substituents introduced at specific, chosen sites, that convert the molecules into probes of the membrane and cell-biological events associated with chemokine action, virus inhibition and the like, as well as for monitoring pharmacokinetics and the like. The detectable labels are preferably attached to the C-terminal region of the bioactive synthetic chemokines of the present invention. A detectable label may be incorporated during synthesis or post-synthesis of the chemokine polypeptide chain. As an example, a detectable label can be incorporated in a pre-ligation peptide segment during chain assembly, e.g., it may be convenient to conjugate a fluorophore to an unprotected reactive group on a resin-bound peptide before removal of other protecting groups and release of the labeled peptide from the resin. Amino acid derivatives comprising a detectable label and chemical synthesis techniques used to incorporate them into a peptide or polypeptide sequence are well known, and can be used for this purpose. In this way the resulting chemokine polypeptide chain ligation product can be designed to contain one or more detectable labels at pre-specified positions of choice. Alternatively, a detectable label can be added to reactive groups, preferably chemoselective reactive groups such as keto or aldehyde groups that permit site-specific attachment, present on a given amino acid of a peptide segment pre-ligation or even the polypeptide chain following ligation.

Detectable labels suitable for this purpose include photoactive groups, as well as chromophores including fluorophores and other dyes, or a hapten such as biotin. Such labels are available from many different commercial sources (See, e.g., Molecular Probes, Oregon USA; Sigma and affiliates, St. Louis Mo., USA; and the like). For on resin labeling, Fluorescein, eosin, Oregon Green, Rhodamine Green, Rhodol Green, tetramethylrhodamine, Rhodamine Red, Texas Red, coumarin and NBD fluorophores, the dabcyl chromophore and biotin are all reasonably stable to hydrogen fluoride (HF), as well as to most other acids, and thus suitable for incorporation via solid phase synthesis. (Peled, et al., Biochemistry (1994) 33:7211; Ben-Efraim, et al., Biochemistry (1994) 33:6966). Other than the coumarins, these fluorophores also are stable to reagents used for de-protection of peptides synthesized using Fmoc chemistry (Strahilevitz, et al., Biochemistry (1994) 33:10951). The t-Boc and α-Fmoc derivatives of ε-dabcyl-L-lysine also can be used to incorporate the dabcyl chromophore at selected sites in a polypeptide sequence. The dabcyl chromophore has broad visible absorption and can used as a quenching group. The dabcyl group also can be incorporated at the N-terminus by using dabcyl succinimidyl ester (Maggiora, et al., J Med Chem (1992) 35:3727). EDANS is a common fluorophore for pairing with the dabcyl quencher in FRET experiments. This fluorophore is conveniently introduced during automated synthesis of peptides by using 5-((2-(t-Boc)-γ-glutamylaminoethyl) amino) naphthalene-1-sulfonic acid (Maggiora, et al., J. Med. Chem. (1992) 35:3727). An α-(t-Boc)-ε-dansyl-L-lysine can be used for incorporation of the dansyl fluorophore into polypeptides during chemical synthesis (Gauthier, et al., Arch Biochem. Biophys. (1993) 306:304). As with EDANS fluorescence of this fluorophore overlaps the absorption of dabcyl. Site-specific biotinylation of peptides can be achieved using the t-Boc-protected derivative of biocytin (Geahlen, et al., Anal. Biochem. (1992) 202:68), or other well known biotinylation derivatives such as NHS-biotin and the like. Racemic benzophenone phenylalanine analog also can be incorporated into peptides following its t-Boc or Fmoc protection (Jiang, et al., Intl. J Peptide Prot. Res. (1995) 45:106). Resolution of the diastereomers can be accomplished during HPLC purification of the products; the unprotected benzophenone also can be resolved by standard techniques in the art. Keto-bearing amino acids for oxime coupling, azalhydroxy tryptophan, biotyl-lysine and D-amino acids are among other examples of amino acids that can be utilized for on resin labeling. It will be recognized that other protected amino acids for automated peptide synthesis can be prepared by custom synthesis following standard techniques in the art. In another embodiment, the bioactive synthetic chemokines of the present invention may include a drug conjugated thereto (See, e.g., WO 00/04926).

D. Synthesis of the Chemokines of the Present Invention And Attachment of Water-Soluble Polymer:

Also provided are methods of producing the bioactive synthetic chemokines of the present invention. The method involves (i) synthesizing an analog of a naturally occurring chemokine that comprises a polypeptide chain having an amino acid sequence that is substantially homologous to the naturally occurring chemokine, where the polypeptide chain is modified at one or more of its N-terminus, N-loop and C-terminus with a moiety selected from an aliphatic chain and an amino acid derivative; and (ii) screening the chemokine analog for antagonist activity compared to the corresponding naturally occurring chemokine.

In particular, the method for production of the N-terminally modified bioactive synthetic chemokines of the present invention comprises: (i) synthesizing an analog of a naturally occurring chemokine that comprises a polypeptide chain having an amino acid sequence that is substantially homologous to the naturally occurring chemokine, where the polypeptide chain is modified at its N-terminus with an aliphatic chain and one or more amino acid derivatives; and (ii) screening the chemokine analog for antagonist activity compared to the corresponding naturally occurring chemokine. The method for production of the C-terminally modified bioactive synthetic chemokines of the present invention comprises: (i) synthesizing an analog of a naturally occurring chemokine that comprises a polypeptide chain having an amino acid sequence that is substantially homologous to the naturally occurring chemokine, where the polypeptide chain is modified at its C-terminus with an aliphatic chain or polycyclic; and (ii) screening the chemokine analog for antagonist activity compared to the naturally occurring chemokine. The method for production of the N-/C-terminally modified bioactive synthetic chemokines of the present invention comprises: (i) synthesizing an analog of a naturally occurring chemokine that comprises a polypeptide chain having an amino acid sequence that is substantially homologous to the naturally occurring chemokine, where the polypeptide chain is modified at its N-terminus with an aliphatic chain and one or more amino acid derivatives, and is modified at its C-terminus with an aliphatic chain or polycyclic; and (ii) screening the chemokine analog for antagonist activity compared to the naturally occurring chemokine.

Synthesis of the bioactive synthetic chemokines of the invention is accomplished by chemical synthesis (i.e., ribosomal-free synthesis), or a combination of biological (i.e., ribosomal synthesis) and chemical synthesis. For chemical synthesis, the bioactive synthetic chemokines of the present invention can be made in toto by stepwise chain assembly or fragment condensation techniques, such as solid or solution phase peptide synthesis using Fmoc and tBoc approaches, or by chemical ligation of peptide segments made in toto by chain assembly, or a combination of chain assembly and biological production. Such stepwise chain assembly or fragment condensation and ligation techniques are well known in the art (See, e.g., Kent, S. B. H., Ann. Rev. Biochem. (1988) 57:957-989; Dawson et al., Methods Enzymol. (1997) 287:3445; Muir et al., Methods Enzymol. (1997) 289:266-298; Wilken et al., Current Opinion In Biotechnology (1998) 9:412-426; Ingenito et al., J. Amer. Chem. Soc. (1999) 121(49):11369-11374; and Muir et al., Chemistry & Biology (1999) 6:R247-R256).

For chemical ligation, a first peptide segment having an N-terminal functional group is ligated to a second peptide segment having a C-terminal functional group that reacts with the N-terminal functional group to form a covalent bond therein between. Depending on the functional groups selected, the ligation reaction generates a product having a native amide bond or a non-native covalent bond at the ligation site. The first or second peptide segment employed for chemical ligation is typically made using stepwise chain assembly or fragment condensation. In particular, when the bioactive synthetic chemokines of the present invention are made by ligation of peptide segments, the segments are made to contain the appropriate pendant chemoselective reactive groups with respect to the intended chemoselective reaction chemistry to be used for ligation. These chemistries include, but are not limited to, native chemical ligation (Dawson, et al., Science (1994) 266:776-779; Kent, et al., WO 96/34878), extended general chemical ligation (Kent, et al., WO 98/28434), oxime-forming chemical ligation (Rose, et al., J. Amer. Chem. Soc. (1994) 116:30-33), thioester forming ligation (Schnölzer, et al., Science (1992) 256:221-225), thioether forming ligation (Englebretsen, et al., Tet. Letts. (1995) 36(48):8871-8874), hydrazone forming ligation (Gaertner, et al., Bioconj. Chem. (1994) 5(4):333-338), and thiazolidine forming ligation and oxazolidine forming ligation (Zhang, et al., Proc. Natl. Acad. Sci. (1998) 95(16):9184-9189; Tam, et al., WO 95/00846).

Reaction conditions for a given ligation chemistry are selected to maintain the desired interaction of the ligation components. For example, pH and temperature, water-solubility of the peptides and components, ratio of peptides, water content and composition of the individual peptides can be varied to optimize ligation. Addition or exclusion of reagents that solubilize the peptides to different extents may further be used to control the specificity and rate of the desired ligation reaction. Reaction conditions are readily determined by assaying for the desired chemoselective reaction product compared to one or more internal and/or external controls.

A preferred method of chemical synthesis employs native chemical ligation, which is disclosed in Kent et al., WO 96/34878, and a method of preparing proteins chemically modified at the N- and/or C-terminal is disclosed in Offord et al., WO 99/11666, the disclosures of which are incorporated herein by reference. In general, a first peptide containing a C-terminal thioester is reacted with a second peptide with an N-terminal cysteine having an unoxidized sulfhydryl side chain. The unoxidized sulfhydryl side chain of the N-terminal cysteine is condensed with the C-terminal thioester in the presence of a catalytic amount of a thiol, preferably benzyl mercaptan, thiophenol, 2-nitrothiophenol, 2-thiobenzoic acid, 2-thiopyridine, and the like. An intermediate peptide is produced by linking the first and second peptides via a β-aminothioester bond, which rearranges to produce a peptide product comprising the first and second peptides linked by an amide bond.

For a combination of chemical and biological production, one peptide segment is made by chemical synthesis while the other is made using recombinant approaches, which segments are then joined using chemical ligation to generate the full-length product. For instance, intein expression systems can be utilized to exploit the inducible self-cleavage activity of an ‘intein’ protein-splicing element to generate a C-terminal thioester peptide segment. In particular, the intein undergoes specific self-cleavage in the presence of thiols such as DTT, b-mercaptoethanol or cysteine, which generates a peptide segment bearing a C-terminal thioester. (See, e.g., Muir et al., Chemistry & Biology (1999) 6:R247-R256; Chong et al., Gene (1997) 192:277-281; Chong et al., Nucl. Acids Res. (1998) 26:5109-5115; Evans et al., Protein Science (1998) 7:2256-2264; and Cotton et al., Chemistry & Biology (1999) 6(9):247-256). This C-terminal thioester bearing peptide segment may then be utilized to ligation a second peptide bearing an N-terminal thioester-reactive functionality, such as a peptide segment having an N-terminal cysteine as employed for native chemical ligation.

The hydrophobic aliphatic chains and amino acid derivatives can be incorporated during chain assembly, post chain assembly or a combination thereof. For incorporation during chain assembly, the amino acid derivatives and/or amino acids having an aliphatic chain attached thereto are incorporated in the stepwise or fragment condensation, and/or the ligation chain assembly process. These amino acids can be added in a stepwise fashion to the growing peptide chain during peptide synthesis, to assembled peptide segments targeted for ligation, or in some instances the pendant N- or C-terminal modifications can be provided by cleavage from a polymer support, whereby the cleavage product yields the desired hydrophobic aliphatic chain. For post chain assembly, amino acids or derivatives thereof having a reactive functional group are incorporated during chain assembly (in protected or unprotected form) which are then utilized in their unprotected reactive form for attachment of the desired hydrophobic moiety, i.e., in a post-peptide synthesis conjugation reaction. The post chain assembly attachment can be performed on a denatured linear peptide chain, or following folding of the polypeptide chain. In a preferred embodiment, the amino acid derivative is added during peptide synthesis at an amino acid position of interest, whereas the N—, C— and/or N-/C-terminal hydrophobic aliphatic chain is added following peptide synthesis through a conjugation reaction. Any of numerous conjugation chemistries can be utilized (See, e.g., Plaue, S et al., Biologicals. (1990) 18(3): 147-57; Wade, J. D. et al., Australas Biotechnol. (1993) 3(6):332-6; Doscher, M. S., Methods Enzymol. (1977) 47:578-617; Hancock, D. C. et al., Mol Biotechnol. (1995) 4(1):73-86; Albericio, F. et al., Methods Enzymol. (1997) 289:313-36), as well as ligation chemistries, depending on the desired covalent linkage. Folding of the bioactive synthetic chemokines of the present invention can be achieved following standard techniques in the art. See, e.g., WO 99/11655; WO 99/11666; Dawson et al., Methods Enymol. (1997) 287:34-45).

For screening the synthesized chemokine compounds for antagonist activity, the compounds are examined by in vitro or in vivo based assays characterized by direct or indirect binding of the chemokine ligand to its corresponding receptor. Examples of chemokine receptors and their corresponding wild type chemokine include CXXXCR1 (Fractalkine); XCR1 (SCM-1); CXCR2 (GRO, LIX, MIP-2); CXCR3 (MIG, IP-10); CXCR4 (SDF-1); CXCR5 (BLC); CCR1 (MIP-1α, RANTES, MCP-3); CCR2 (MCP-1, MCP-3, MCP-5); CCR3 (Eotaxin, RNATES, MIP-1α); CCR4 (MDC, TARC); CCR5 (RANTES, MIP-1α1β; MIP-1β; CCR6 (MIP-3α); CCR7 (SLC, MIP-3β); CCR8 (TCA-3); and CCR9 (TECK). In vitro and in vivo assays for these systems are well know, and readily available or can be created de novo. See, e.g., U.S. Pat. No. 5,652,133; U.S. Pat. No. 5,834,419; WO 97/44054; WO 00/04926; and WO 00/0492. For instance, natural, transformed, and/or transgenic cell lines expressing one or more chemokine receptors are typically used to monitor the effect of chemokine-induced chemotaxis or the inhibition of this event when exposed to a bioactive synthetic chemokine of the present invention. Animal models also may be employed, for example, to monitor a response profile in conjunction with treatment with a bioactive synthetic chemokine of the present invention, or to characterize the pharmacokinetic and pharmacodynamic properties of the compounds. To characterize the compounds of the invention as inhibitors of viral infection, envelope-mediated cell fusion assays employing a target cell line and an envelop cell line may be employed for screening bioactive synthetic chemokines of the present invention for their ability to prevent HIV infection. Of course, cell-free viral infection assays may be employed as well for this purpose.

As an example, for assessing antagonism of chemotaxis in general, peripheral blood leukocytes can be employed, such as those isolated from normal donors according to established protocols for purification of monocytes, T lymphocytes and neutrophils. A panel of C, CC, CXXXC and CXC chemokine receptor-expressing test cells can be constructed and evaluated following exposure to serial dilutions of individual compounds of the invention. Native chemokines can be used as controls. For instance, a panel of cells transfected with expression cassettes encoding various chemokine receptors are suitable for this purposes. For instance, antagonist of chemokines such as RANTES, SDF-1α or SDF-1β and MIP can be screened using tranformants expression CXCR4/Fusion/LESTR, CCR3, CCR5, CXC4 (such cells are available from various commercial and/or academic sources or can be prepared following standard protocols; see, e.g., Risau, et al., Nature 387:671-674 (1997); Angiololo, et al., Annals NY Acad. Sci. (1996) 795:158-167; Friedlander, et al., Science (1995) 870:1500-1502). The results can be expressed as the chemotaxis index (“CI”) representing the fold increase in the cell migration induced by stimuli versus control medium, and statistical significance determined.

Receptor binding assays also can be performed, for example, to evaluate competitive inhibition versus receptor recycling effects (see, Signoret, N. et al., “Endocytosis and recycling of the HIV coreceptor CCR5,” J. Cell Biol. 2000 151(6):1281-94; Signoret, N. et al., “Analysis of chemokine receptor endocytosis and recycling,” Methods Mol. Biol. 2000;138:197-207; Pelchen-Matthews, A. et al., “Chemokine receptor trafficking and viral replication,” Immunol Rev. 1999 April;168:33-49; Daugherty, B. L. et al., “Radiolabeled chemokine binding assays,” Methods Mol. Biol. 2000; 138:129-34; Mack, M. et al. “Downmodulation and recycling of chemokine receptors,”Methods Mol. Biol. 2000;138:191-5; all herein incorporated by reference). This approach is well known and typically will employ labeled bioactive synthetic chemokines of the present invention in the presence of increasing concentrations of unlabeled native chemokines following standard protocols. Of course labeling can be on either or both ligands. In this type of assay, the binding data can be analyzed, for example, with a computer program such as LIGAND (P. Munson, Division of Computer Research and Technology, NIH, Bethesda, Md.), and subjected to Scatchard plots analysis with both “one site” and “two site” models compared to native leukocytes or the panel of receptor-transfected cells expressing a target chemokine receptor. The rate of competition for binding by unlabeled ligands can then be calculated with the following formula: % inhibition=1−(Binding in the presence of unlabeled chemokine/binding in the presence of medium alone)×100.

For screening the compounds for their ability to prevent or alleviate viral infection and disease, the compounds can be screened against a panel of cells stably expressing either the appropriate receptor exposed to various viral strains and controls. For instance, U87/CD4 cells expressing CCR3, CCR5, CXC4 or CXCR4 receptors can be employed for screening infection of M-tropic, T-tropic and dual tropic HIV strains. Inhibition of viral infection can be accessed as a percentage of infection relative to the concentration of chemokine and control concentrations. See., e.g., McKnight, et al., Virology (1994) 201:8-18); and Mosier, et al., Science (1993) 260:689-692; Simmons, et al, Science (1997) 276:276-279; Wu, et al., J. Exp. Med. (1997) 185:168-169; and Trkola, et al., Nature (1996) 384:184-186). Calcium mobilization assays are another example useful for screening for antagonists of receptor binding, for instance to identify antagonists of native chemokines that are chemotactic for neutrophils and eosinophils (Jose, et al., J. Exp. Med. 179:881-887 (1994)). As another example, angiogenic activities of compounds of the invention can be evaluated by the chick chorioallantoic membrane (CAM) assay (Oikawa, et al., Cancer Lett. (1991) 59:57-66.

The bioactive synthetic chemokines of the present invention have many uses, including use as research tools, diagnostics and as therapeutics. In particular, the bioactive synthetic chemokines of the present invention have been found to possess valuable pharmacological properties, and have been shown to effectively block the inflammatory effects associated with the corresponding wild type molecules—which are involved in various disorders including asthma, allergic rhinitis, atopic dermatitis, atheroma/atheroschleosis, organ transplant rejection, and rheumatoid arthritis. Accordingly, they are useful for the treatment of asthma, allergic rhinitis, atopic dermatitis, atheroma/atheroschleosis, organ transplant rejection, and rheumatoid arthritis. For instance, several of the bioactive synthetic chemokines of the present invention such as the RANTES and SDF-1α or SDF-1β antagonists also have been shown to inhibit HIV-1 infection, and antagonists (e.g., vMIP-II analogues) can be used for the same purpose. Thus, the RANTES, or SDF-1α or SDF-1β antagonists and the vMIP-II analogues of the invention can be used for inhibiting HIV-1 in mammals. The potential of the compounds for utility against HIV-1 is determined by the method, described in the following Examples. The potential of the compounds for utility against inflammatory effects is determined by methods well known to those skilled in the art. Moreover, it will be understood that the bioactive synthetic chemokines of the present invention can be utilized alone, or in combination with each other, as well as in combination with other non-chemokine drugs that are synergistic in treating a given disorder.

By way of example, and not by way of limitation, the following are some specific examples of wild type chemokines molecules and their associated biological properties to illustrate the general utility of making the bioactive synthetic chemokines of the present invention. For instance, SCM-1 is a C-Chemokine expressed in spleen. It is substantially related to the CC and CXC-Chemokines, with a primary difference being that it only has the second and fourth of the four cysteines conserved in these proteins (Yoshida et al. FEBS Letters (1995) 360(2):155-159); Yoshida et al. J. Biol. Chem. (1998) 273(26):16551-16554). In humans, there are two highly homologous SCM-1 proteins, SCM-1α and SCM-1β, which differ by two amino acid substitutions. SCM-I is found to be about 60% identical with lymphotactin, a murine lymphocyte-specific chemokine. SCM-1 and lymphotactin may thus represent the human and murine prototypes of C-Chemokines or Gamma-Chemokines. Both SCM-I molecules specifically induce migration in murine L1.2 cells engineered to express the orphan receptor, GPR5, which is expressed primarily in placenta, and weakly in spleen and thymus among various human tissues. Accordingly, antagonists of SCM-1 find use in blocking the normal function of GPR4.

As another example, the soluble from of Fractalkine, a 76 amino acid CXXXC-chemokine, is a potent chemoattractant for T-cells and monocytes but not for neutrophils. Fractalkine is increased markedly after stimulation with INF or L1. The human receptor for Fractalkine is designated CX3CR1. The receptor mediates both the adhesive and migratory functions of Fractalkine. The human receptor is expressed in neutrophils, monocytes, T-lymphocytes, and several solid organs, including brain. The receptor has been shown to function with CD4 as a coreceptor for the envelope protein from a primary isolate of HIV-1. A cell-cell fusion assay demonstrates that Fractalkine potently and specifically inhibits fusion. (See, e.g., Bazan et al Nature (1997) 385(6617):640-644; Combadiere et al. J. Biol. Chem. (1998) 273(37):23799-23804; Rossi et al. Genomics (1998) 47(2):163-170; and Faure et al. Science (2000) 287:2274-2277). It is therefore apparent that antagonists of Fractalkine can find use in the treatment of various arthritic disorders involving the TNF or IL1 pathway, such as arthritis, as well as finding use as a blocker of HIV infection.

Eotaxin is an additional example. This protein is 74 amino acids in length, and is classified as a CC-Chemokine due to its characteristic cysteine pattern. It has been found in the bronchoalveolar lavage of guinea pigs used as a model of allergic inflammation, and implicated in asthma-related disorders. Eotaxin induces substantial eosinophil accumulation at a 1-2 pM dose in the skin without significantly affecting the accumulation of neutrophils. Eotaxin is a potent stimulator of both guinea pig and human eosinophils in vitro. The factor appears to share a binding site with RANTES on guinea pig eosinophils. Eotaxin induces a calcium flux response in normal human eosinophils, but not in neutrophils or monocytes. The response cannot be desensitized by pretreatment of eosinophils with other CC-Chemokines. In basophils Eotaxin induces higher levels of chemotactic response than RANTES, but it only has a marginal effect on either histamine release or leukotriene C4 generation. It also may play a role in chemotaxis of B-cell lymphoma cells. The primary receptor for Eotaxin is CCR3. (See, e.g., Bartels et al., Biochem. Biophys. Res. Comm. (1996) 225(3):1045-51); Jose et al., J. Exp. Med. (1994) 179:881-887); Ponath et al., J. Clin. Investigation (1996) 97(3):604-612); Ponath et al., J. Exp. Med. (1996) 183(6):2437-2448); Yamada et al., Biochem. Biophys. Res. Comm. (1997) 231(2):365-368). Accordingly, antagonists of Eotaxin can be used as potent modulators of asthma and other eosinophil related allergic disorders.

RANTES is another example of a target chemokine for which antagonists are of particular interest. It is a CC-Chemokine involved in many disorders ranging from inflammation, organ rejection to HIV infection. The synthesis of RANTES is induced by TNF-alpha and IL1-alpha, but not by TGF-beta, IFN-gamma and IL6. RANTES is produced by circulating T-cells and T-cell clones in culture but not by any T-cell lines tested so far. The expression of RANTES is inhibited following stimulation of T-lymphocytes. RANTES is chemotactic for T-cells, human eosinophils and basophils and plays an active role in recruiting leukocytes into inflammatory sites. RANTES also activates eosinophils to release, for example, eosinophilic cationic protein. It changes the density of eosinophils and makes them hypodense, which is thought to represent a state of generalized cell activation and is associated most often with diseases such as asthma and allergic rhinitis. RANTES also is a potent eosinophil-specific activator of oxidative metabolism. RANTES increases the adherence of monocytes to endothelial cells. It selectively supports the migration of monocytes and T-lymphocytes expressing the cell surface markers CD4 and UCHL1. These cells are thought to be pre-stimulated helper T-cells with memory T-cell functions. RANTES activates human basophils from some select basophil donors and causes the release of histamines. On the other hand RANTES can also inhibit the release of histamines from basophils induced by several cytokines including one of the most potent histamine inducers, MCAF.

RANTES has been shown recently to exhibit biological activities other than Chemotaxis. It can induce the proliferation and activation of killer cells known as CHAK (C—C-Chemokine-activated killer), which are similar to cells activated by IL2. RANTES is expressed by human synovial fibroblasts and may participate in the ongoing inflammatory process in rheumatoid arthritis. High affinity receptors for RANTES (approximately 700 binding sites/cell; Kd=700 picoM) have been identified on the human monocytic leukemia cell line THP-1, which responds to RANTES in chemotaxis and calcium mobilization assays. The chemotactic response of THP-1 cells to RANTES is markedly inhibited by pre-incubation with MCAF (monocyte chemotactic and activating factor) or MIP-1-alpha (macrophage inflammatory protein). Binding of RANTES to monocytic cells is competed for by MCAF and MIP-1-alpha. Receptors for RANTES are CCR1, CCR3 and CCR5. The clinical use and significance of antagonists of RANTES is multifold. For instance, antibodies to natural RANTES can dramatically inhibit the cellular infiltration associated with experimental mesangioproliferative nephritis. In addition, natural RANTES appears to be expressed highly in human renal allografts undergoing cellular rejection related to transplant rejection of the kidney (Pattison et al., Lancet (1994) 343(8891): 209-11 (1994). Chemically modified forms of RANTES (Aminooxypentane-RANTES or AOP-RANTES; and n-nonanoyl-RANTES or NNY-RANTES) have been shown to act as an antagonist for the CCR-5 receptor of chemokines and to have the ability to inhibit HIV-1 infection. Accordingly, the antagonist N—, C— and N-/C-terminal modified analogs of RANTES according to present invention are useful as an anti-inflammatory agent in the treatment of diseases such as asthma, allergic rhinitis, atopic dermatitis, organ transplant, atheroma/atherosclerosis and rheumatoid arthritis.

Antagonists of the chemokines SDF-1α: and β are additional examples, which belong to the CXC class of chemokines. SDF-1β differs by having four additional amino acids at the C-terminus. These chemokines are more than 92% identical to their non-human counterparts. SDF-1 is expressed ubiquitously with the exception of blood cells. SDF-1 acts on lymphocytes and monocytes, but not neutrophils in vitro and is a highly potent chemoattractant for mononuclear cells in vivo. It also induces intracellular actin polymerization in lymphocytes. SDF-1 acts both in vitro and in vivo as a chemoattractant for human hematopoietic progenitor cells, giving rise to mixed types of progenitors, and more primitive types. SDF-1 also appears to be involved in ventricular septum formation. Chemotaxis of CD34+cells is increased in response to a combination of SDF-1 and IL-3. SDF has been shown also to induce a transient elevation of cytoplasmic calcium in these cells. A primary receptor for SDF-1 is CXCR4, which also functions as a major T-lymphocyte coreceptor for HIV 1. See, e.g., Aiuti et al, J. Exp. Med (1997) 185(1):111-120 (1997); Bleul et al., J. Exp. Med. (1996) 184(3):1101-1109 (1996); Bleul et al., Nature (1996) 382(6594):829-833; D'Apuzzo et al. European J. Immunol. (1997) 27(7):1788-1793; Nagasawa et al., Nature (1996) 382:635-638); Oberlin et al., Nature (1996) 382(6594):833-835. So for instance, the SDF-1α or SDF-1β antagonists of the present invention are useful as an anti-inflammatory agent in the treatment of diseases such as asthma, allergic rhinitis, atopic dermatitis, atheroma/atherosclerosis and rheumatoid arthritis. Moreover, the SDF-1α or SDF-1β antagonists of the invention can be used alone or in combination with other compounds, such as the RANTES antagonist analogs of the invention, for blocking the effects of SDF-1, RANTES, MIP-1α, and/or MIP-1β, in mammals with respect to the recruitment and/or activation of pro-inflammatory cells, or treating or blocking HIV-1 infection.

Accordingly, another aspect of the invention relates to pharmaceutical compositions and methods of treating a mammal in need thereof by administering therapeutically effective amounts of compounds comprising one or more chemokine(s) of the present invention, or pharmaceutically acceptable salts thereof. By “pharmaceutically acceptable salt” is intended to mean a salt that retains the biological effectiveness and properties of the polypeptides of the invention and which are not biologically or otherwise undesirable. Salts may be derived from acids or bases. Acid addition salts are derived from inorganic acids, such as hydrochloric acid, hydrobromic acid, sulfuric acid (giving the sulfate and bisulfate salts), nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, salicylic acid, p-toluenesulfonic acid, and the like. Base addition salts may be derived from inorganic bases, and include sodium, potassium, lithium, ammonium, calcium, magnesium salts, and the like. Salts derived from organic bases include those formed from primary, secondary and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. Preferred organic bases are isopropylamine, diethylamine, ethanolamine, piperidine, tromethamine, and choline.

The term “treatment” as used herein covers any treatment of a disease in a mammal, particularly a human, and includes: (i) preventing the disease from occurring in a subject which may be predisposed to the disease but has not yet been diagnosed as having it; (ii) inhibiting the disease, i.e. arresting its development; or (iii) relieving the disease, i.e. causing regression of the disease.

By the term “a disease state in mammals that is prevented or alleviated by treatment with a bioactive synthetic chemokine of the present invention” as used herein is intended to cover all disease states which are generally acknowledged in the art to be usefully treated with bioactive synthetic chemokines of the present invention in general, and those disease states which have been found to be usefully prevented or alleviated by treatment with the specific compounds of the invention. These include, by way of illustration and not limitation, asthma, allergic rhinitis, atopic dermatitis, viral diseases, atheroma/atheroschleosis, rheumatoid arthritis and organ transplant rejection.

As used herein, the term “therapeutically effective amount” refers to that amount of a bioactive synthetic chemokine of the present invention which, when administered to a mammal in need thereof, is sufficient to effect treatment (as defined above), for example, as an anti-inflammatory agent, anti-asthmatic agent, an immunosuppressive agent, or anti-autoimmune disease agent to inhibit viral infection in mammals. The amount that constitutes a “therapeutically effective amount” will vary depending on the chemokine derivative, the condition or disease and its severity, and the mammal to be treated, its weight, age, etc., but may be determined routinely by one of ordinary skill in the art with regard to contemporary knowledge and to this disclosure. As used herein, the term “q.s.” means adding a quantity sufficient to achieve a stated function, e.g., to bring a solution to a desired volume (e.g., 100 mL).

The chemokines of this invention and their pharmaceutically acceptable salts, i.e., the active ingredient, are administered at a therapeutically effective dosage, i.e., that amount which, when administered to a mammal in need thereof, is sufficient to effect treatment, as described above. Administration of the bioactive synthetic chemokines of the present invention described herein can be via any of the accepted modes of administration for agents that serve similar utilities. As used herein, the terms “bioactive synthetic chemokines of the present invention”, “[pharmaceutically acceptable salts of the] polypeptides of the invention” and “active ingredient” are used interchangeably.

The level of the bioactive synthetic chemokines of the present invention present in a formulation can vary within the full range employed by those skilled in the art, e.g., from about 0.01 percent weight (% w) to about 99.99% w of the bioactive synthetic chemokine of the present invention based on the total formulation and about 0.01% w to 99.99% w excipient. More typically, the bioactive synthetic chemokines of the present invention will be present at a level of about 0.5% w to about 80% w.

While human dosage levels have yet to be optimized for the bioactive synthetic chemokines of the present invention, generally, a daily dose is from about 0.05 to 25 mg per kilogram body weight per day, and most preferably about 0.01 to 10 mg per kilogram body weight per day. Thus, for administration to a 70 kg person, the dosage range would be about 0.07 mg to 3.5 g per day, preferably about 3.5 mg to 1.75 g per day, and most preferably about 0.7 mg to 0.7 g per day. The amount of antagonist administered will, of course, be dependent on the subject and the disease state targeted for prevention or alleviation, the nature or severity of the affliction, the manner and schedule of administration and the judgment of the prescribing physician. Such use optimization is well within the ambit of those of ordinary skill in the art.

Administration can be via any accepted systemic or local route, for example, via parenteral, oral (particularly for infant formulations), intravenous, nasal, bronchial inhalation (i.e., aerosol formulation), transdermal or topical routes, in the form of solid, semi-solid or liquid or aerosol dosage forms, such as, for example, tablets, pills, capsules, powders, liquids, solutions, emulsion, injectables, suspensions, suppositories, aerosols or the like. The bioactive synthetic chemokines of the present invention can also be administered in sustained or controlled release dosage forms, including depot injections, osmotic pumps, pills, transdermal (including electrotransport) patches, and the like, for the prolonged administration of the polypeptide at a predetermined rate, preferably in unit dosage forms suitable for single administration of precise dosages. The compositions will include a conventional pharmaceutical carrier or excipient and a bioactive synthetic chemokine of the present invention and, in addition, may include other medicinal agents, pharmaceutical agents, carriers, adjuvants, etc. Carriers can be selected from the various oils, including those of petroleum, animal, vegetable or synthetic origin, for example, peanut oil, soybean oil, mineral oil, sesame oil, and the like. Water, saline, aqueous dextrose, and glycols are preferred liquid carriers, particularly for injectable solutions. Suitable pharmaceutical carriers include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. Other suitable pharmaceutical carriers and their formulations are described in “Remington's Pharmaceutical Sciences” by E. W. Martin.

If desired, the pharmaceutical composition to be administered may also contain minor amounts of non-toxic auxiliary substances such as wetting or emulsifying agents, pH buffering agents and the like, such as for example, sodium acetate, sorbitan monolaurate, triethanolamine oleate, etc.

Although more of the active ingredient may be required, oral administration can be used to deliver the bioactive synthetic chemokines of the present invention using a convenient daily dosage regimen, which can be adjusted according to the degree of prevention desired or in the alleviation of the affliction. For such oral administration, a pharmaceutically acceptable, non-toxic composition is formed by the incorporation of any of the normally employed excipients, such as, for example, pharmaceutical grades of mannitol, lactose, starch, povidone, magnesium stearate, sodium saccharine, talcum, cellulose, croscarmellose sodium, glucose, gelatin, sucrose, magnesium carbonate, and the like. Such compositions take the form of solutions, suspensions, dispersible tablets, pills, capsules, powders, sustained release formulations and the like. Oral formulations are particularly suited for treatment of gastrointestinal disorders. Oral bioavailablity for general systemic purposes can be adjusted by utilizing excipients that improve uptake to systemic circulation, such as formulation comprising acetylated amino acids. See, e.g., U.S. Pat. No. 5,935,601 and U.S. Pat. No. 5,629,020.

The compositions may take the form of a capsule, pill or tablet and thus the composition will contain, along with the active ingredient, a diluent such as lactose, sucrose, dicalcium phosphate, and the like; a disintegrant such as croscarmellose sodium, starch or derivatives thereof; a lubricant such as magnesium stearate and the like; and a binder such as a starch, polyvinylpyrrolidone, gum acacia, gelatin, cellulose and derivatives thereof, and the like.

Liquid pharmaceutically administrable compositions can, for example, be prepared by dissolving, dispersing, etc. a bioactive synthetic chemokine of the present invention (about 0.5% to about 20%) and optional pharmaceutical adjuvants in a carrier, such as, for example, water, saline, aqueous dextrose, glycerol, glycols, ethanol, preservatives and the like, to thereby form a solution or suspension. If desired, the pharmaceutical composition to be administered may also contain minor amounts of nontoxic auxiliary substances such as wetting agents, suspending agents, emulsifying agents, or solubilizing agents, pH buffering agents and the like, for example, sodium acetate, sodium citrate, cyclodextrine derivatives, polyoxyethylene, sorbitan monolaurate or stearate, etc. Actual methods of preparing such dosage forms are known, or will be apparent, to those skilled in this art; for example, see Remington's Pharmaceutical Sciences, Mack Publishing Company, Easton, Pa. The composition or formulation to be administered will, in any event, contain a quantity of the active ingredient in an amount effective to prevent or alleviate the symptoms of the subject being treated. For oral administration to infants, a liquid formulation (such as a syrup or suspension) is preferred.

For a solid dosage form containing liquid, the solution or suspension, in for example propylene carbonate, vegetable oils or triglycerides, is preferably encapsulated in a gelatin capsule. For a liquid dosage form, the solution, e.g. in a polyethylene glycol, may be diluted with a sufficient quantity of a pharmaceutically acceptable liquid carrier, e.g. water, to be easily measured for administration.

Alternatively, liquid or semi-solid oral formulations may be prepared by dissolving or dispersing the active ingredient in vegetable oils, glycols, triglycerides, propylene glycol esters (e.g. propylene carbonate) and the like, and encapsulating these solutions or suspensions in hard or soft gelatin capsule shells.

In applying the bioactive synthetic chemokines of the present invention to treatment of the above conditions, administration of the active ingredients described herein are preferably administered parenterally. Parenteral administration is generally characterized by injection, either subcutaneously, intramuscularly or intravenously, and can include intradermal or intraperitoneal injections as well as intrasternal injection or infusion techniques. Injectables can be prepared in conventional forms, either as liquid solutions or suspensions, solid forms suitable for solution or suspension in liquid prior to injection, as emulsions or in biocompatible polymer-based microspheres (e.g., liposomes, polyethylene glycol derivatives, poly(D,C)lactide and the like). Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, protein carriers and the like, such as for example, sodium acetate, polyoxyethylene, sorbitan monolaurate, triethanolamine oleate, cyclodextrins, serum albumin etc.

The bioactive synthetic chemokines of the present invention can be administered parenterally, for example, by dissolving such molecules in a suitable solvent (such as water or saline) or incorporation in a liposomal formulation followed, by dispersal into an acceptable infusion fluid. A typical daily dose of a polypeptide of the invention can be administered by one infusion, or by a series of infusions spaced over periodic intervals. For parenteral administration there are especially suitable aqueous solutions of an active ingredient in water-soluble form, for example in the form of a water-soluble salt, or aqueous injection suspensions that contain viscosity-increasing substances, for example sodium carboxymethylcellulose, sorbitol and/or dextran, and, if desired, stabilizers. The active ingredient, optionally together with excipients, can also be in the form of a lyophilisate and can be made into a solution prior to parenteral administration by the addition of suitable solvents.

A more recently devised approach for parenteral administration employs the implantation of a slow-release or sustained-release system, such that a constant level of dosage is maintained. See, e.g., U.S. Pat. No. 3,710,795, U.S. Pat. No. 5,714,166 and U.S. Pat. No. 5,041,292, which are hereby incorporated by reference.

The percentage of the active ingredient contained in such parental compositions is highly dependent on the specific nature thereof, as well as the activity of the polypeptide and the needs of the subject. However, percentages of active ingredient of 0.01% to 10% in solution are employable, and will be higher if the composition is a solid which will be subsequently diluted to the above percentages. Preferably the composition will comprise 0.02-8% of the active ingredient in solution.

Another method of administering the bioactive synthetic chemokines of the present invention utilizes both a bolus injection and a continuous infusion. This is a particularly preferred method when the therapeutic treatment is for the prevention of HIV-1 infection.

Aerosol administration is an effective means for delivering the bioactive synthetic chemokines of the present invention directly to the respiratory tract. Some of the advantages of this method are: 1) it circumvents the effects of enzymatic degradation, poor absorption from the gastrointestinal tract, or loss of the therapeutic agent due to the hepatic first-pass effect; 2) it administers active ingredients which would otherwise fail to reach their target sites in the respiratory tract due to their molecular size, charge or affinity to extra-pulmonary sites; 3) it provides for fast absorption into the body via the alveoli of the lungs; and 4) it avoids exposing other organ systems to the active ingredient, which is important where exposure might cause undesirable side effects. For these reasons, aerosol administration is particularly advantageous for treatment of asthma, local infections of the lung, and other diseases or disease conditions of the lung and respiratory tract.

There are three types of pharmaceutical inhalation devices, nebulizers inhalers, metered-dose inhalers and dry powder inhalers. Nebulizer devices produce a stream of high velocity air that causes the chemokine derivative (which has been formulated in a liquid form) to spray as a mist which is carried into the patient's respiratory tract. Metered-dose inhalers typically have the formulation packaged with a compressed gas and, upon actuation, discharge a measured amount of the polypeptide by compressed gas, thus affording a reliable method of administering a set amount of agent. Dry powder inhalers administer the polypeptide in the form of a free flowing powder that can be dispersed in the patient's air-stream during breathing by the device. In order to achieve a free flowing powder, the chemokine derivative is formulated with an excipient, such as lactose. A measured amount of the chemokine derivative is stored in a capsule form and is dispensed to the patient with each actuation. All of the above methods can be used for administering the present invention.

Pharmaceutical formulations based on liposomes are also suitable for use with the chemokines of of this invention. See, e.g., U.S. Pat. No. 5,631,018, U.S. Pat. Nos. 5,723,147, and 5,766,627. The benefits of liposomes are believed to be related to favorable changes in tissue distribution and pharmacokinetic parameters that result from liposome entrapment of drugs, and may be applied to the polypeptides of the present invention by those skilled in the art. Controlled release liposomal liquid pharmaceutical formulations for injection or oral administration can also be used.

For systemic administration via suppository, traditional binders and carriers include, for example, polyethylene glycols or triglycerides, for example PEG 1000 (96%) and PEG 4000 (4%). Such suppositories may be formed from mixtures containing the active ingredient in the range of from about 0.5 w/w % to about 10 w/w %; preferably from about 1 w/w % to about 2 w/w %.

As described above, and further illustrated in the specific Examples that follow, the bioactive synthetic chemokines of the present invention find use as antagonist of the naturally occurring chemokines. In particular, the bioactive synthetic chemokines of the present invention having enhanced potency as an antagonist find use in the analysis and treatment of various disease states, such as asthma, allergic rhinitis, atopic dermatitis, organ transplant rejection, viral diseases, atheroma/atheroschleosis, rheumatoid arthritis and organ transplant rejection. The bioactive synthetic chemokines of the present invention also can be utilized in designing and screening small molecule antagonist of their cognate receptors. For instance, the structural diversity engineered into the compounds of the invention facilitates a more rational approach in the design, screening and fine tuning of better small molecule compounds for use as medicaments in the treatment of diseases involving the natural activity of chemokine receptors.

### EXAMPLES

The following preparations and examples are given to enable those skilled in the art to more clearly understand and to practice the present invention. They should not be considered as limiting the scope of the invention, but merely as being illustrative and representative thereof.

### Abbreviations

DIEA diisopropylethyleamine
DMF N,N-dimethylformamide
DNP 2,4-dinitrophenyl
GuHCl guanidinium hydrochloride
HBTU O-(1H-benzotriazol-1-yl)-1,1,3,3-tetramethyl-uronium hexafluorophosphate
HF hydrogen fluoride
TFA trifluoroacetic acid
Aib aminoisobutyric acid
Hyp hydroxyproline
Tic 1,2,3,4-tetrahydroisoquinoline-3-COOH
Indol indoline-2-carboxylic acid
P(4,4DiF) 4-difluoro-proline
Thz L-thiazolidine-4-carboxylic acid
Hop L-homoproline
ΔPro 3,4-dehydro-proline
F(3,4-DiOH) 3, 4dihydroxyphenylalanine
F(3,4-DiOH, pBzl)) pBzl,-3, 4dihydroxyphenylalanine
p-Bz benzophenone
Cha cyclohexyl-alanine
βNal 3-(2-naphtyl)-alanine
Chg cyclohexyl-glycine
Phg phenylglycine
HoF homophenylalanine
F(F)₅ pentafluorophenylalanine
tBuA tert-butylalanine
F(4-Me) 4-methylphenylalanine
tL tert-leucine
CycP 1-amino-1-cyclopentanecarboxylic acid
CycH 1-amino-1-cyclohexanecarboxylic acid
Nle norleucine
Aminooxypentane-RANTE(2-68) AOP-RANTES
n-Nonanoyl-RANTES(2-68) NNY-RANTES

### Example 1

### General Synthesis Approach for the Chemokines of the Present Invention

Peptides for bioactive synthetic chemokines of the present invention were made by solid-phase peptide synthesis. Solid-phase synthesis was performed on a custom-modified 430A peptide synthesizer from Applied Biosystems, using in situ neutralization/2-(1H-benzotriazol-1-yl)-1,1,1,3,3-tetramethyluronium hexa fluorophosphate activation protocols for stepwise Boc chemistry chain elongation (Schnolzer, et al., Int. J. Peptide Protein Res. (1992) 40:180-193). The N-terminal peptide fragments were synthesized on a thioester-generating resin. The resin was split after attachment of the residue preceding the position investigated (elongation from C to N terminus) and the peptide elongated manually on a 0.03 mmol scale. Each synthetic cycle consisted of Nα-Boc-removal by a 1 to 2 minute treatment with neat TFA, a 1-min DMF flow wash, a 10- to 20-minute coupling time with 1.0 mmol of preactivated Boc-amino acid in the presence of excess DIEA and a second DMF flow wash. Nα-Boc-amino acids (1.1 mmol) were preactivated for 3 minutes with 1 mmol HBTU (0.5M in DMF) in the presence of excess DIEA (3 mmol). After each manual coupling step, residual free amine was evaluated with the ninhydrin assay (Sarin, et al., Anal Biochem. (1981) 117:147-157). The C-terminal fragment comprising amino acids were synthesized on a standard —O—CH₂-phenylacetamidomethyl resin. After chain assembly was completed, the peptides were deprotected and cleaved from the resin by treatment with anhydrous HF for 1 hour at 0° C. with 5% p-cresol as a scavenger. In all cases, the imidazole side chain DNP protecting groups remained on His residues because the DNP-removal procedure is incompatible with C-terminal thioester groups. However DNP was gradually removed by thiols during the ligation reaction, yielding unprotected His. After cleavage, both peptides were precipitated with ice cold diethylether, dissolved in aqueous acetonitrile and lyophilized. The peptides were purified by RP-HPLC with a C18-column from Waters by using linear gradients of buffer B (acetonitile/10% H₂O/0.1% trifluoroacetic acid) in buffer A (H₂O/0.1% trifluoroacetic acid) and UV detection at 214 nm. Samples were analyzed by electrospray mass spectrometry with a Platform II instrument (Micromass, Manchester, England). Peptides were utilized for ligation to generate full-length chemokine polypeptide chains using native chemical ligation (Dawson, et al., Science (1994) 266:776-779); Wilken, et al., Chem. Biol. (1999) 6:43-51; and Camarero, et al., Current Protocols in Protein Science (1999) 18.4.1-18.4.21). Folding of the polypeptide chains was accomplished in the presence of Cys-SH/(Cys-S)₂ following standard techniques (Wilken et al., Chem. Biol. (1999) 6:43-51).

### Example 2

### Synthesis of N—, C— and N-/C-terminal analogs of NNY-RANTES, AOP-RANTES, and SDF-1

Analogs of RANTES (1-68) and SDF-10 (1-72) were prepared as in Example 1 and described herein to illustrate a general approach of making CC and CXC chemokines of the present invention. In particular, N-terminal, C-terminal and N-/C-terminal modified RANTES analogs were based on modifications to the chemokine compound CH₃—(CH₂)₇—C(O)—RANTES (2-68), also referred to as n-nonanoyl-RANTES (2-68) or “NNY-RANTES”, and the chemokine compound CH₃—(CH₂)₄—O—N═CH—CO-RANTES (2-68), also referred to as aminooxypentane-RANTES or “AOP-RANTES”. The NNY-RANTES, AOP-RANTES and additional RANTES derivative molecules utilized for this purpose are described in WO 99/11666, which reference is incorporated herein by reference. The N—, C— and N-/C-terminal analogs of SDF-1 were constructed using the same basic design approach as for the RANTES analogs.

For the N-terminal modifications to a given target chemokine, such as the NNY and AOP modifications to RANTES, chemical variants were prepared as described above and in WO 99/11666 and Wilken et al., Chem. Biol. (1999) 6:43-51, utilizing on-resin elaboration of the N-terminal peptide segment employed for ligation to generated the pendant N-terminal modification (e.g., NNY or AOP), followed by cleavage/deprotection, purification and use of the unprotected N-terminal modified peptide α-thioester in native chemical ligation to the C-terminal peptide segment to form the full length product. Peptides were synthesized and amino acid substitutions, including amino acid derivatives, were incorporated during peptide synthesis as described in Example 1. Native chemical ligation as in Example 1 was utilized to generate the linear product, where ligation was at the Lys³¹-Cys³² site for the RANTES analogs, and for the SDF-1 analogs at the Asn³³-Cys³⁴ site. Equimolar amount of peptide fragments (2-2.5 mM) were dissolved in 6M GuHCl, 100 mM phosphate, pH 7.5, 1% benzylmercaptan, and 3% thiophenol. The reactions usually were carried out overnight. The resulting polypeptide products were purified and analyzed as described above for peptide segments. For generating the folded protein, the purified polypeptide chains of NNY-RANTES analogs (about 0.5 to 1 mg/mL) were dissolved in 2M GuHCl, 100 mM Tris, pH 8.0 containing 8 mM cysteine, 1 mM cystine and 10 mM methionine. After gentle stirring overnight, the protein solution was purified by RP-HPLC as described above. Other folding conditions were used in the case of SDF-1 analogs: SDF-1 and Met⁰-SDF-1 were oxidized at 0.5 mg/mL in 1M GuHCl, 0.1M Tris, pH8.5 at room temperature in the presence of air. After stirring overnight, folding was complete. AOP—, caproyl- and NNY-SDF-1 were oxidized in the same buffer but in the presence of 2M GuHCl.

For chemical conjugation of the fatty acid to a given folded protein, two basic steps were involved. First, the fatty acid was functionalized with an amino oxy group. Second, a reactive carbonyl group was introduced specifically in the carboxyl-terminal domain of the protein, a region believed not to be critical for the activity of chemokines. For this purpose, chemokine analogs targeted for C-terminal fatty acid modification were synthesized with a C-terminal Lys(Ser)Gly sequence extension. Thus, for example, NNY-RANTES (2-68) was synthesized to contain a Lys(Ser)Gly sequence extension at the C-terminus. The reactive carbonyl group was generated by NaIO₄ treatment of the refolded protein, thus allowing the site-specific attachment of the fatty acid moiety through a stable oxime bond.

For fatty acid functionalization, 0.2 mmol fatty acid (palmitate, oleate, arachidonate, cholate) was activated with equimolar amounts of DCC and HOAt in 0.5 ml of DMF/DCM mixture (1:1, v:v) and added to a 0.5 ml DMF solution of 0.25 mmol Boc-AoA-NH—(CH₂)₂—NH₂ and the apparent pH adjusted to pH. 8.0 with N-ethylmorpholine. For the cholesteryl derivative, 0.2 mmol cholesteryl-chloroformate was dissolved in 0.5 ml DCM and added to an ethanolic solution of 0.25 mmol Boc-AoA-NH—(CH₂)₂—NH₂ and the apparent pH adjusted to pH 9.0 with triethylamine. After overnight incubation the volatiles were removed under vacuum and the product isolated either by flash chromatography or by preparative HPLC on a C4 column The Boc group was removed by TFA treatment and the product verified by ESI-MS.

For protein oxidation, the target protein (2 mg/mL) was dissolved in a 0.1 M sodium phosphate buffer, pH7.5 containing 6M guanidine chloride and methionine added to get a 100-fold molar excess of scavenger over protein. A 10-fold excess of sodium periodate was then added and the solution incubated for 10 min in the dark. The reaction was stopped by the addition of a 1000-fold molar excess ethylene glycol over periodate and the solution further incubated for 15 min at room temperature. The solution was then dialyzed against 0.1% acetic acid and finally lyophilized. For example, oxidation of the C-terminal lateral serine was shown to be almost quantitative by ESI-MS, where a mass of 8141.1±0.7% was obtained in the case of AOP-RANTES-K(S)G, corresponding to the loss of 31 Da to form the glyoxylyl derivative and no peak corresponding to the mass of the starting material was observed.

Conjugation of the fatty acid with the chemokine was accomplished in 0.1 M sodium acetate buffer, pH 5.3, in the presence of 0.1% sarcosyl, 20 mM methionine and a 20-fold-excess of functionalized fatty acid over the protein. After agitation for 16-20 h at 37° C., the conjugate, as an oxime bond formed between the amino-oxy group of the fatty acid and the chemokine aldehyde, was purified using reverse phase-HPLC and the product characterized by ESI-MS. For all analogs, the coupling of aminooxy-functionalized fatty acids to oxidized protein was almost quantitative as controlled by analytical HPLC.

### Example 3

### N-terminal Analogs of NNY- and AOP-RANTES

For the N-terminal RANTES derivatives, the modifications were made to one or more of the N-terminal region of amino acids corresponding to the first eight amino acid residues of NNY-RANTES (2-68) or AOP-RANTES (2-68), which first eight amino acid residues have the following sequence —PYSSDTTP—. These correspond to amino acid residues 2-9 of the 68 amino acid residue wild type RANTES polypeptide chain (i.e., RANTES (1-68)) shown in FIGS. 10A-10D, since the first residue (Ser) of naturally occurring RANTES (1-68) is replaced by the n-nonanoyl substituent in NNY-RANTES (2-68) and aminooxypentane in AOP-RANTES (2-68). So for example, a substitution in NNY-RANTES (2-68) at amino acid position 2 is indicated below by the general compound formula “NNY-P2X-RANTES (3-68)”, where NNY is n-nonanoyl, X is an amino acid substituted for the proline (P) at position 2 of NNY-RANTES (2-68), and RANTES (3-68) represents the remaining 66 amino acids of NNY-RANTES (2-68), as read in the N— to C-terminal direction. By way of another example, a substitution in NNY-RANTES (2-68) at amino acid position 3 is indicated by the general compound formula “NNY-P-Y3X-RANTES (4-68)”, where NNY is n-nonanoyl, X is an amino acid substituted for the tyrosine (Y) at position 3 of NNY-RANTES (2-68), and RANTES (4-68) represents the remaining 65 amino acids of NNY-RANTES (2-68), as read in the N- to C-terminal direction. For multiply substituted AWY-RANTES analogs, the following example of a compound formula for three substitutions in NNY-RANTES (2-68) at amino acid positions 2, 3 and 9 is indicated by the general compound formula “NNY-P2X-Y3X—SSDTT-P9X-RANTES (10-68)”, where ANY is n-nonanoyl, X is the same or different amino acid substituted for the proline (P) at position 2, tyrosine (Y) at position 3, and proline (P) 9 of NNY-RANTES (2-68), SSDTT corresponds to amino acids 4-8 of NNY-RANTES (2-68), and RANTES (10-68) represents the remaining 59 amino acids of NNY-RANTES (2-68), as read in the N— to C-terminal direction.

The following are examples of the NNY-P2X-RANTES (3-68) analogs prepared.

| Compound | Number |
|---|---|
| NNY-P2Aib-RANTES (3-68) | 1 |
| NNY-P2Hyp-RANTES (3-68) | 2 |
| NNY-P2Tic-RANTES (3-68) | 3 |
| NNY-P2Indol-RANTES (3-68) | 4 |
| NNY-P2P(4,4DiF)-RANTES (3-68) | 5 |
| NNY-P2Thz-RANTES (3-68) | 6 |
| NNY-P2HoP-RANTES (3-68) | 7 |
| NNY-P2ΔPro-RANTES (3-68) | 8 |
| NNY-P2A-RANTES (3-68) | 9 |

The following are examples of the NNY-P-Y3X-RANTES (4-68) analogs prepared.

| Compound | Number |
|---|---|
| NNY-P-Y3P-RANTES (4-68) | 10 |
| NNY-P-Y3A-RANTES (4-68) | 11 |
| NNY-P-Y3L-RANTES (4-68) | 12 |
| NNY-P-Y3V-RANTES (4-68) | 13 |
| NNY-P-Y3F(3,4-DiOH)-RANTES (4-68) | 14 |
| NNY-P-Y3F(3,4-DiOH,pBzl)-RANTES (4-68) | 15 |
| NNY-P-Y3pBz-RANTES (4-68) | 16 |
| NNY-P-Y3Cha-RANTES (4-68) | 17 |
| NNY-P-Y3βNal-RANTES (4-68) | 18 |
| NNY-P-Y3Chg-RANTES (4-68) | 19 |
| NNY-P-Y3Phg-RANTES (4-68) | 20 |
| NNY-P-Y3Hof-RANTES (4-68) | 21 |
| NNY-P-Y3F(F)₅-RANTES (4-68) | 22 |
| NNY-P-Y3tbuA-RANTES (4-68) | 23 |
| NNY-P-Y3F(4-Me)-RANTES (4-68) | 24 |
| NNY-P-Y3tL-RANTES (4-68) | 25 |
| NNY-P-Y3CycP-RANTES (4-68) | 26 |
| NNY-P-Y3CycH-RANTES (4-68) | 27 |
| NNY-P-Y3Nle-RANTES (4-68) | 28 |

The following compounds are examples of the NNY-PY-S4X-RANTES (5-68) analogs prepared.

| Compound | |
|---|---|
| NNY-PY-S4A-RANTES (5-68) | 29 |
| NNY-PY-S4tbuA-RANTES (5-68) | 30 |

The following compounds are examples of the NNY-PYS-S5X-RANTES (6-68) analogs prepared.

| Compound | Number |
|---|---|
| NNY-PYS-S5tbuA-RANTES (6-68) | 31 |

The following compounds are examples of the NNY-PYSS-D6X-RANTES (7-68) analogs prepared.

| Compound | Number |
|---|---|
| NNY-PYSS-D6tbuA-RANTES (7-68) | 32 |

The following compounds are examples of the NNY-PYSSD-T7X-RANTES (8-68) analogs prepared.

| Compound | Number |
|---|---|
| NNY-PYSSD-T7tbuA-RANTES (8-68) | 33 |

The following compounds are examples of the NNY-PYSSDT-T8X-RANTES (9-68) analogs prepared.

| Compound | Number |
|---|---|
| NNY-PYSSDT-T8tBuA-RANTES (9-68) | 34 |

The following compounds are examples of the NNY-PYSSDTT-P9X-RANTES analogs prepared.

| Compound | Number |
|---|---|
| NNY-PYSSDTT-P9Hyp-RANTES (10-68) | 35 |
| NNY-PYSSDTT-P9Aib-RANTES (10-68) | 36 |
| NNY-PYSSDTT-P9ΔPro-RANTES (10-68) | 37 |
| NNY-PYSSDTT-P9Thz-RANTES (10-68) | 38 |

The following compounds are examples of the double substituted analogs NNY-P2X-Y3X-RANTES (4-68), and triple substituted analogs NNY-P2X-Y3X—SSDTT-P9X-RANTES (10-68) prepared.

| Compound | Number |
|---|---|
| NNY-P2Hyp-Y3tButA-RANTES (4-68) | 39 |
| NNY-P2Thz-Y3tButA-RANTES (4-68) | 40 |
| NNY-P2Hyp-Y3Chg-RANTES (4-68) | 41 |
| NNY-P2Thz-Y3Chg-RANTES (4-68) | 42 |
| NNY-P2Thz-Y3Chg-SSDTT-P9Aib-RANTES (10-68) | 43 |

### Example 4

### N-terminal, N-loop Analogs of NNY-RANTES

The following compounds are intended to be illustrative of additional NNY-substituted-RANTES analogs in which the N-loop (residues 12-20 of RANTES) is modified to increase potency towards CCR5 without affecting signal transduction via CCR1 and CCR3.

For the N-terminal, N-loop RANTES analogs, the N-loop modifications were made to NNY-RANTES (2-68), where the N-loop corresponds to amino acids 12-20. The N-loop of RANTES has the amino acid sequence —FAYIARPLP— (SEQ ID NO.: 29). So for example, a substitution in NNY-RANTES (2-68) at amino acid position 12 has the general compound formula “NNY-PYSSDTTPCC-F12pBz-RANTES (13-68)”, where NNY is n-nonanoyl, PYSSDTTPCC corresponds to amino acids 2-11 of RANTES (1-68), F12pBz indicates substitution of the amino acid derivative pBZ for the phenylalanine (F) at position 12 of RANTES (1-68), and RANTES (13-68) represents the remaining amino acid residues 13-68 of RANTES (1-68), as read in the N— to C-terminal direction.

| Compound | Number |
|---|---|
| NNY-PYSSDTTPCC-F12pBz-RANTES (13-68) | 44 |
| NNY-PYSSDTTPCC-F12Y-RANTES (13-68) | 45 |
| NNY-PYSSDTTPCC-F12F(4-Me)-RANTES (13-68) | 46 |
| NNY-PYSSDTTPCC-F12(4-F)-RANTES (13-68) | 47 |
| NNY-PYSSDTTPCCF-A13R-RANTES (14-68) | 48 |
| NNY-PYSSDTTPCCF-A13S-RANTES (14-68) | 49 |
| NNY-PYSSDTTPCCFA-Y14F-RANTES (15-68) | 50 |
| NNY-PYSSDTTPCCFA-Y14Cha-RANTES (15-68) | 51 |
| NNY-PYSSDTTPCCFAY-I15tBuA-RANTES (16-68) | 52 |
| NNY-PYSSDTTPCCFAY-I15S-RANTES (16-68) | 53 |
| NNY-PYSSDTTPCCFAYI-A16S-RANTES (17-68) | 54 |
| NNY-PYSSDTTPCCFAYA-R17A-RANTES (18-68) | 55 |
| NNY-PYSSDTTPCCFAYA-R17H-RANTES (18-68) | 56 |
| NNY-PYSSDTTPCCFAYAR-P18Thz-RANTES (19-68) | 57 |
| NNY-PYSSDTTPCCFAYARP-L19I-RANTES (20-68) | 58 |
| NNY-PYSSDTTPCCFAYARP-L19Cha-RANTES (20-68) | 59 |
| NNY-PYSSDTTPCCFAYARPL-P20Thz-RANTES (21-68) | 60 |

### Example 5

### N-terminal RANTES Analogs of NNY-RANTES

The following compounds are intended to be illustrative of additional NNY-substituted-RANTES analogs in which a different hydrophobic aliphatic chain was employed in lieu of the NNY substituent.

| Compound | Number |
|---|---|
| CH2═CH—CH2—CH2—CH2—CH2—CH2—CH2—CO | 61 |
| RANTES (2-68) | |
| Nle-Met-RANTES (1-68) | 62 |

| Compound | Number |
|---|---|
| Dodecanoyl-RANTES (3-68) | 63 |
| Lauryl-Hyp-RANTES (3-68) | 64 |

| Compound | Number |
|---|---|
| Myristoyl-RANTES (4-68) | 65 |
| Dodecanoyl-Hyp-RANTES (4-68) | 66 |

### Example 6

### C-terminal and N/C-terminal Analogs of NNY- and AOP-RANTES

AOP- and NNY-RANTES having a Lys-Gly C-terminal extension, with the epsilon amino group of the Lys acylated by a serine residue were prepared. These derivatives were conjugated, after periodate oxidation of the serine extension, with aminooxyacetyl-functionalized compounds including fluorophores (FITC, NBD, Cy-5 and BODIPY-FI) or lipids. These C-terminally labeled chemokines retain their biological properties and introduction of a aliphatic moiety as like as CH₃—(CH₂)₁₄—CONH—(CH₂)₂—NHCO—CH₂—O—NH₂ was shown to improve the potency of the chemokine. In order to find out the most effective compound, different fatty acids and lipids were functionalized with an aminooxy group by coupling with Boc-AoA-NH—(CH₂)₂—NH₂, followed by Boc removal laurate, palmitate, oeate, eicosanoate, cholic acid, and cholesteryl-chloroformate. One or more of these derivatives were conjugated to oxidized NNY-RANTES-K(S)G or AOP-RANTES-K(S)G, where the AOP analogs are exemplified below:

| Compound | Number |
|---|---|
| AOP-RANTES-K(lauryl)-G | 67 |
| where “(lauryl)” is an abbreviation for gloxylyl = AoA-ethylene | |
| diamine-laurate and so on | |
| AOP-RANTES-K(palmitoyl)-G | 68 |
| AOP-RANTES-K(eicosanoyl)-G | 69 |
| AOP-RANTES-K(oleoyl)-G | 70 |
| AOP-RANTES-K(cholyl)-G | 71 |
| AOP-RANTES-K(cholesteryl)-G | 72 |

Chemical variants of the lipidic moiety were also prepared by another strategy. Such compounds were synthesized by on-resin elaboration of the C-terminal segment by attachment of the fatty acid to the Fmoc-deprotected Boc-peptide-Lys-Gly-resin, prior to cleavage, purification and use in chemical ligation to form the full length polypeptide.

In designing these compounds, there were two main reasons that the lipid coupling was utilized. First, there is now more and more evidence that the anti HIV-1 inhibitory activity of the RANTES compounds is related to the ability to down-regulate the receptor. This means that once internalized the ligand-receptor complex which should be normally dissociated in early endosomes with recycling of the receptor could also interact with the plasma membrane or some cytoplasmic fatty acid binding proteins. Accordingly, lipid modification of the ligand may retarget the complex to a specific intracellular subdomain simply through hydrophobic interactions and thus delaying the recycling of the receptor. Several recent papers dealing with intracellular protein trafficking support the idea that acylation is a common mechanism of increasing the affinity of proteins for detergent resistant membranes and may be the primary targeting mechanism for proteins without membrane spans (See, e.g., Melkonian et al., J. Biol. Chem. (1999) 274:3910-3917; Zlatkine et al., J Cell Sci. (1997) 110:673-679; Zhan et al. Cancer Immunol. Immunother. (1998) 46:55-60). Second, the modification also was carried out to change the pharmacokinetic properties of the compounds. Several recent papers support this concept (see, e.g., Honeycutt et al. Pharm. Res. (1996) 13:1373-1377; Kurtzhals et al. J Pharm. Sci. (1997) 86:1365-1368; Markussen et al. Diabetologia (1996) 39:281-288).

As demonstrated in the Examples that follow, the enhancement of activity was surprising and unexpected, since the modification was intended to change pharmacokinetics. An expected result would have been that the activity decreased, but the hoped-for improvement in pharmacokinetics would have nevertheless given an acceptable compromise.

### Example 7

### N-terminal Analogs SDF-1α of SDF-1β

The following N-terminal SDF-1β (1-72) derivatives were prepared to illustrate a general approach of making a CXC chemokine of the present invention. By way of example, the N-terminus of SDF-1β was modified to generate compounds having hydrophobic aliphatic chain at the N-terminus. Compounds that further include an amino acid derivative at the N-terminal region, and/or an aliphatic chain at the C-terminal region are prepared as described above for the RANTES compounds. In particular, suitable N-terminal substituents were prepared and tested that included, by way of illustration and not limitation Lys, Met-Lys, caproyl-Lys, CH₃—(CH₂)₇—C(O) and CH₃—(CH₂)₄—O—NH-glyoxylyl. The following compounds are examples of some of the SDF-1α and SDF-1β analogs prepared.

| Compound | Number |
|---|---|
| Lys-SDF-1 (2-72) | 73 |
| Met-Lys-SDF-1 (2-72) | 74 |
| Caproyl-Lys-SDF-1 (2-72) | 75 |
| NNY-SDF-1 (2-72) | 76 |
| AOP-glyoxylyl-SDF-1 (2-72) | 77 |

### Example 8

### Screening Assays

Several of the RANTES and SDF analogs prepared in Examples 3-7 and others were screened for antagonist activity, using an HIV-based assay to characterize the blocking function for this particular indication for which RANTES and SDF-1 find use. In general, the compounds were passed through a preliminary screen for their ability to inhibit HIV envelope-mediated cell fusion. The most promising of these compounds were subsequently tested for their ability to inhibit cell-free viral infection of a target cell line. These assays were chosen since the cell fusion assay and the in vitro cell-free viral infection assay have been found to be useful indicators of potency in vivo, as determined in the SCID mouse model (Mosier et al., J. Virol. (1999) 73:3544-3550). Moreover, since the increase in anti-viral potency of NNY-RANTES over AOP-RANTES has been found to be due to factors other than an increase in affinity for CCR5, the compounds were evaluated in terms of activity in the cell fusion assay, rather than affinity for CCR5.

### Example 9

### Envelope-Mediated Cell Fusion Assays

The ability of a given panel of compounds of Examples 3-7 to inhibit CCR5-dependent cell fusion was determined using cells engineered to viral envelop proteins fusing with cells bearing CD4 and CCR5 and containing a reporter system. CCR5-tropic viral envelope-mediated cell fusion assays were carried out essentially as described in Simmons et al. (Science (1997) 276:276-279) using the cell lines HeLa-P5L and HeLa-Env-ADA, both of which were kindly provided by the laboratory of M. Alizon (Paris). Briefly, HeLa-P5L cells were seeded in 96-well plates (10⁴ cells per well in 100 μl). Twenty-four hours later medium was removed and medium containing 10⁴ HeLa-Env-ADA cells per well plus chemokines was added (200 μl final volume). After a further twenty-four hours, cells were washed once in PBS and lysed in 50 μl PBS/0.5% NP-40 for 15 min at room temperature. Lysates were assayed for for β-galactosidase activity by the addition of 50 μl 2× CPRG substrate (16 mM chlorophenol red-β-D-galactopyranoside; 120 mM Na₂HPO₄, 80 mM NaH₂PO₄, 20 mM KCl, 20 mM MgSO₄, and 10 mM β-mercaptoethanol) followed by incubation for 1-2 hours in the dark at room temperature. The absorbance at 575 nm was then read on a Labsystems microplate reader. From these values, percentage inhibition [100×(OD₍ₜₑₛₜ₎−OD_{(negative control)})/OD_{(positive control)}−OD_{(negative control))}] was calculated at each inhibitor concentration. A plot of percentage fusion inhibition against inhibitor concentration allowed the calculation of IC₅₀ values for each compound.

Significantly, a majority of the compounds tested exhibited greater potency relative to wild type RANTES. Results for selected RANTES antagonist analogs are shown in Table 1 below.

TABLE 1
Cell-Fusion Screen

| | |
|---|---|
| Compound Number | Mean Relative Potency |

N-terminal modified NNY-RANTES

| | |
|---|---|
| 19 | 7 |
| 23 | 7 |
| 40 | 4 |
| 42 | 2 |
| NNY-RANTES (control) | 18-25 |

N-loop modified NNY-RANTES

| | |
|---|---|
| 54 | 15 |
| 57 | 15 |
| 58 | 13 |
| 59 | 14 |
| NNY-RANTES (control) | 18-25 |

C-Terminal modified AOP-RANTES

| | |
|---|---|
| 68 | 45 |
| AOP-RANTES (Control) | 100 |

In Table 1, for the mean relative potencies, absolute values for IC₅₀ s in the fusion assay vary across experiments performed on different days, although rank orders of activity remain constant. In order to normalize results, AOP-RANTES was used as a control in each experiment. So the IC₅₀s in each experiment were expressed relative to that of AOP-RANTES, which was given an arbitrary value of 100. Although most all of the compounds tested exhibited greater potency relative to wild type RANTES, potencies of certain compounds, such as compound numbers 19, 23, 40 and 42, were such that the more than 50% inhibition was obtained even at the lowest dilution in the series.

### Example 10

### Cell-Free Viral Infection Assays

The cell-free viral infection assays were carried out in the same way as the envelope-mediated cell fusion assay, except that in this case the envelope cell line was replaced by live R5-tropic virus. HEK293-CCR5 cells (7, kindly provided by T. Schwartz, Copenhagen) were seeded into 24 well plates (1.2×10⁵ cells/well). After overnight incubation, competition binding was performed on whole cells for 3 h at 4° C. using 12 pM [¹²⁵I]MIP-1-α (Amersham) plus variable amounts of unlabelled ligand in 0.5 ml of ‘Binding Buffer’ (50 mM HEPES, pH 7.4, supplemented with 1 mM CaCl₂, 5 mM MgCl₂, and 0.5% (w/v) bovine serum albumin). After incubation, cells were washed rapidly four times in ice cold Binding Buffer supplemented with 0.5 M NaCl. Cells were lysed in 1 ml 3 M Acetic Acid, 8 M Urea and 2% NP-40. Lysed material was counted for 1 minute using a Beckman Gamma 4000 scintillation counter. Determinations were made in duplicate and IC50 values were derived from monophasic concentration inhibition curves fitted using Prism software. Table 2 illustrates the increase in potency over NNY-RANTES shown in the preliminary screen by compound numbers 19 and 23.

TABLE 2
In Vitro Infectivity Data For Selected Compounds
From Cell-Free Viral Infection Assay

| | AOP- | NNY- | Compound | Compound |
|---|---|---|---|---|
| | RANTES | RANTES | 23 | 19 |
| Experimental | 140 | 32 | 17 | 15 |
| IC₅₀ | 47 | 8 | 3.8 | 34 |
| Infectivity | 260 | 26 | 28 | 9.9 |
| Results | 135 | 30 | 14 | 12 |
| Average | 145 pM | 24 pM | 14 pM | 12 pM |
| infectivity | | | | |
| IC₅₀ | | | | |
| Cell-fusion | 480 pM | 97 pM | 38 pM | 26 pM |
| result for | | | | |
| comparison | | | | |

### Example 11

### Combination Treatment with anti-CCR5 and anti-CXCR4 Compounds

The following example illustrates the protective effects of employing an anti-CCR5 (e.g., NNY-RANTES) and an anti-CXCR4 (e.g., SDF-1 antagonist or AMD 3100) in combination for blocking HIV infection, and blocking the potential conversion of R5 strains of HIV to X4 strains. A SCID mouse model was utilized for the purpose. In particular, the protective effects of NNY-RANTES and AMD 3100 (a small organic molecule anti-X4 agent) were tested in SCID mice, repopulated with human peripheral blood leukocytes and challenged with HIV-1 following the methods described in Mosier, Adv. Immunol. (1996) 63:79-125; Picchio, et al., J. Virol. (1997) 71:7124-7127; Picchio, et al., J. Virol. (1998) 72:2002-2009; and Offord et al., WO 99/11666. NNY-RANTES was administered as in Table X, and AMD 3100 used as a 200 mg/ml solution. Challenge was with an R5 HIV virus except for the AMD 3100 group alone. No escape mutants were observed in the combination therapy, and all of the appropriately treated mice remained virus free throughout the experiment. This indicates that the N—, C— and N-/C-terminal RANTES derivatives of the invention can be used in combination with anti-X4 strain compounds such as AMD 3100 or SDF—I antagonist, such as those described herein, for blocking HIV infection in mammals.

### Example 12

### Preparation of RANTES Analog G1755-01

The RANTES analog, G1755-01, depicted in FIG. 11 was synthesized as follows.

1. Preparation of AOP-[RANTES(2-33)]-thioester Sequence:

(SEQ ID NO.: 30)

aminooxypetane-glyoxalyl-PYSSDTTPCC
FAYIARPLPR AHIKEYFYTS GK-thioester

The N-terminal peptide segment AOP-[RANTES(2-33)]-αthioester (thioester also depicted as —C(O)SR) containing an N-terminal aminooxypentane-glyoxalyl oxime moiety (AOP) was synthesized by assembly of the RANTES(2-33) sequence on a thioester generating resin (Hackeng, et al., PNAS (1999) 96: 10068-10073), and then modified at the N-terminus by direct coupling of an aminooxypentane-glyoxalyl oxime moiety [i.e. CH₃(CH₂)₄—O—N═CHCOOH] to the resin following standard protocols (Wilken et al., Chemistry & Biology (1999) 6(1):43-51). The peptide-resin was cleaved with hydrogen fluoride and purified by reverse-phase HPLC to yield the α-carboxy thioester peptide AOP-RANTES(2-33)-thioester. Observed mass=4179.64 Da; Calculated mass=4178.57 Da (average isotope composition).

2. Preparation of [RANTES(34-68)]-Lys69(GP6)-Leu70 Sequence:

| | |
|---|---|
| CSNPAVVFVT RKNRQVCANP EKKWVREYIN | (SEQ ID NO.: 31) |
| SLEMSK(GP6)L | |

where GP6 is bound to the εN of Lys 69:

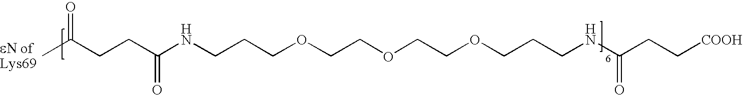

a. Solid Phase Peptide and On-{Peptide Resin} Water-soluble Polymer Synthesis

The C-terminal peptide segment [RANTES(34-68)]-Lys69(Fmoc)-Leu70 was synthesized by conventional solid phase peptide synthesis on a Boc-Leu-OCH₂-Pam-resin using the in situ neutralization/HBTU activation protocols for Boc chemistry solid phase peptide synthesis as described in Schnolzer, et al, Int. J. Pep. Protein Res. 40:180-193.

After completion of chain assembly, the Fmoc group on the epsilon nitrogen of Lys69 was removed from the protected peptide-resin with 20% piperidine in DMF treatment. Succinic anhydride (Succ) was then coupled in a HOBT (hydroxybenzotriazole) solution in DMF containing DIEA to the epsilon nitrogen on Lys69. After washing, the free carboxyl group of the resin-bound succinic acid was activated by addition of carbonyldiimidazole in DMF. After another wash cycle, 50% (4,7,10)-trioxatridecane-1,13diamine (TTD) in 0.5M HOBT in DMF was added. After washing, the resin-bound polymer was ready for the next cycle of succinic anhydride coupling/CDI activation/TTD coupling. After 6 cycles, succinic anhydride was coupled to the last TTD amine in HOBT/DMF=DIEA solution to yield the Lys69 modified compound having an amide linked -(Succ-TTD)₆-Succ-OH polymer.

The peptide/linear water-soluble polymer resin was cleaved from the resin with hydrogen fluoride and the product purified by reverse-phase preparative HPLC to yield [RANTES(34-68)]-Lys69(GP6)-Leu70. Observed mass=6253 Da; Calculated mass=6252 Da (average isotope composition).

3. Preparation of G1755-01 Sequence:

(SEQ ID NO.: 32)

aminooxypentane-glyoxalyl-PYSSDTTPCC
FAYIARPLPR AHIKEYFYTS GKCSNPAVVF VTRKNRQVCA
NPEKKWVREY INSLEMSK(GP6)L

The N-terminal and C-terminal fully unprotected peptide segments described above were joined together by native chemical ligation as described Dawson et al, Science 266:776-779 (1994). The full-length polypeptide product in reduced form was purified using reverse-phase HPLC with a linear gradient of acetonitrile versus water containing 0.1% trifluoroacetic acid. Observed mass=10,060 Da.

The full-length polypeptide with GP6 attached was folded with concomitant formation of 2 disulfide bonds in aqueous buffer containing a cysteine-cystine redox couple, and purified by reverse-phase HPLC following standard protocols (Wilken et al., Chemistry & Biology (1999) 6(1):43-51). The folded product was homogeneous on HPLC. Observed mass=10,056 Da; Calculated mass=10,058 Da (average isotope composition).

### Example 13

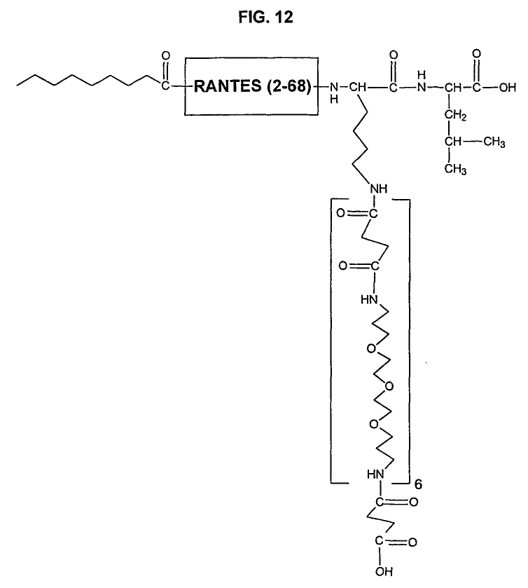

### Preparation of RANTES Analog G1755 The G1755 compound depicted in FIG. 12 was synthesized as follows.

1. Preparation of n-nonanoyl-RANTES(2-33) Sequence:

(SEQ ID NO.: 33)

n-nonanoyl-PYSSDTTPCC FAYIARPLPR AHIKEYFYTS
GK-thioester

n-nonanoyl-RANTES(2-33) was synthesized on a thioester-producing resin as described above, and then modified at the N-terminus by direct coupling of n-nonanoic acid to the resin. The peptide resin was cleaved with hydrogen fluoride and purified by reverse-phase BPLC to yield n-nonanoyl-RANTES(2-33). Observed mass=4177 Da; Calculated mass=4177.62 Da (average isotope composition).

2. Preparation of RANTES(34-68)-Lys69(GP6)-Leu70 Sequence:

| | |
|---|---|
| CSNPAVVFVT RKNRQVCANP EKKWVREYIN | (SEQ ID NO.: 34) |
| SLEMSK(GP6)L | |

a. Solid Phase Peptide and On-{Peptide Resin} Water-soluble Polymer Synthesis

RANTES(34-68)-Lys69(Fmoc)-Leu70 was synthesized by conventional solid phase peptide synthesis on a Boc-Leu-OCH₂-Parn-resin using the in situ neutralization/HBTU activation protocols for Boc chemistry solid phase peptide synthesis as described above.

After completion of chain assembly the Fmoc group on the epsilon nitrogen of Lys69 was removed from the protected peptide-resin and the GP6 construct was synthesized on the peptide resin as described above. The peptide/linear water soluble polymer resin was cleaved with hydrogen fluoride and the product purified by reverse-phase preparative HPLC to yield RANTES(34-68)-Lys69(GP6)-Leu70. Observed mass=6252.87 Da; Calculated mass=6252.24 Da (average isotope composition).

3. Preparation of G1755 Seqeunce:

(SEQ ID NO.: 35)

nonanoyl-PYSSDTTPCC FAYIARPLPR AHIKEYFYTS
GKCSNPAVVF VTRKNRQVCA NPEKKWVREY
INSLEMSK(GPG)L

a. Assembly of Full-Length Polypeptide By Native Chemical Ligation

The N-terminal and C-terminal fully unprotected peptide segments were joined together by native chemical ligation as described above in Example 18. The full-length polypeptide in reduced form was purified using reverse-phase HPLC with a linear gradient of acetonitrile versus water containing 0.1% trifluoroacetic acid. Observed mass=10060.74 Da; Calculated mass=10058.67 Da (average isotope composition).

b. Folding and Purification of Polypeptide Modified with Water-soluble Polymer

The full-length polypeptide with GP6 attached was folded with concomitant formation of 2 disulfide bonds in aqueous buffer containing a cysteine-cystine redox couple, and purified by reverse-phase HPLC as described above. The folded product was homogeneous on HPLC. Observed mass=10057.43 Da; Calculated mass 10054.67 Da (average isotope composition).

### Example 14

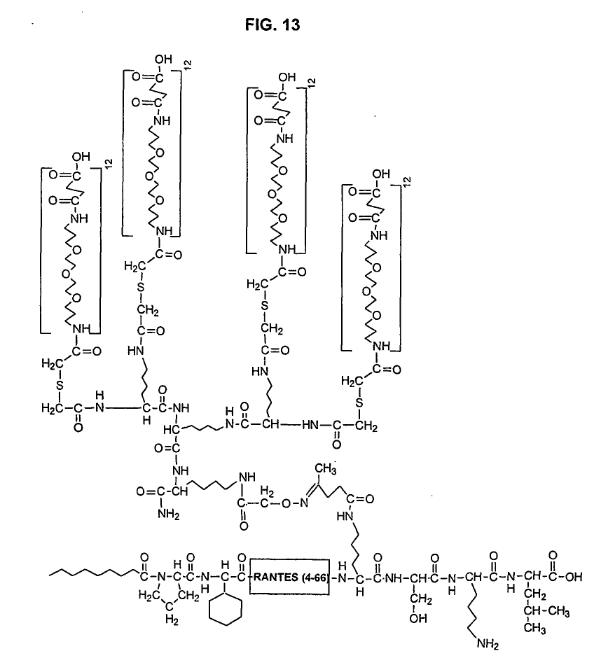

### Preparation of RANTES Analog G1805 The G1805 compound depicted in FIG. 13 was synthesized as follows.

1. Preparation of n-nonanoyl-RANTES(2-33)(Tyr3Chg)-thioester Sequence:

(SEQ ID NO.: 36)

nonanoyl-PXSSDTTPCC FAYIARPLPR AHIKEYFYTS
GK-thioester

where X=L-Cyclohexylglycine (Chg)
n-nonanoyl-RANTES(2-33)(Tyr3Chg) was synthesized on a thioester-producing resin as described above, and then modified at the N-terminus by direct coupling of n-nonanoic acid to the resin. The peptide resin was cleaved with hydrogen fluoride and purified by reverse-phase HPLC to yield n-nonanoyl-RANTES(2-33)(Tyr3Chg). Observed mass 4151.98 Da; Calculated mass=4152.6 Da (average isotope composition).

2. Preparation of RANTES(34-68)(Met67Lys(Lev))-Lys69-Leu70 Sequence:

| | |
|---|---|
| CSNPAVVFVT RKNRQVCANP EKKWVREYIN | (SEQ ID NO.: 37) |
| SLEK(Lev)SKL | |

RANTES(34-68)(Met67Lys)-Lys69(Fmoc)-Lue70 was synthesized by conventional solid phase peptide synthesis on a Boc-Leu-OCH₂-Pam-resin using the in situ neutralization/HBTU activation protocols for Boc chemistry solid phase peptide synthesis as described above.

After completion of chain assembly the Fmoc group on the epsilon nitrogen of Lys67 was removed from the protected peptide-resin with 20% piperidine in DMF treatment. Levulinic acid was activiated as the symmetrical anhydride with 1,3-di-isopropylcarbodi-imide and coupled to the epsilon nitrogen of Lys67. The peptide-resin was cleaved with hydrogen fluoride and the product purified by reverse-phase preparative HPLC to yield RANTES(34-68)(Met67Lys(Lev))-Lys69-Leu70. Observed mass=4433.22 Da; Calculated mass=4434.19 Da (average isotope composition).

3. Preparation of G1805 Sequence:

(SEQ ID NO.: 38)

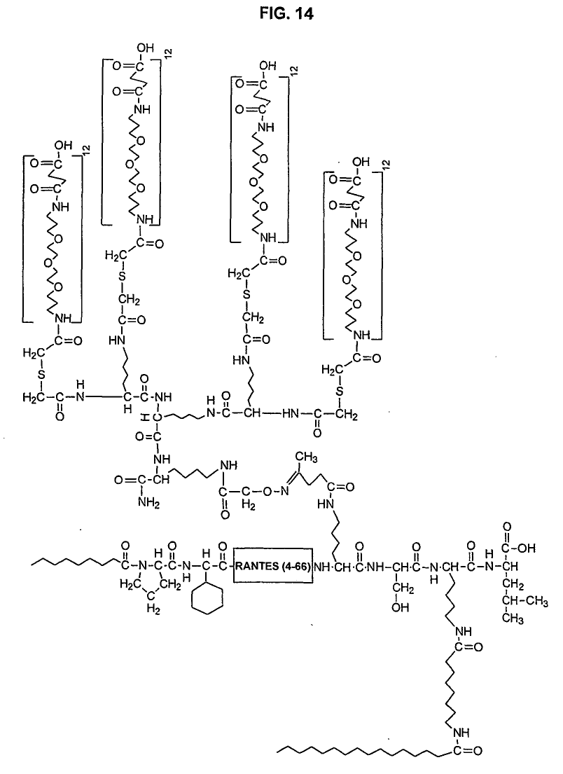

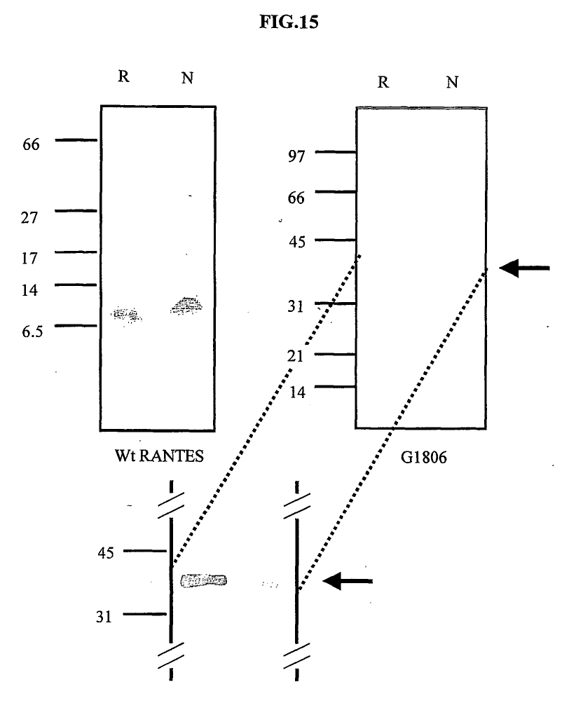

nonanoyl-PXSSDTTPCC FAYIARPLPR AHIKEYFYTS
GKCSNPAVVF VTRKNRQVCA NPEKKWVREY
INSLEK(Lev-GP29)SKL

where X=L-Cyclohexylglycine (Chg), and where GP29=branched oxime-linked water-soluble polymer construct depicted in FIGS. 13 and 14.

a. Assembly of Full-Length Polypeptide By Native Chemical Ligation

The N-terminal and C-terminal fully unprotected peptide segments were joined together by native chemical ligation as described above. The full-length polypeptide in reduced form was purified using reverse-phase HPLC with a linear gradient of acetonitrile versus water containing 0.1% trifluoroacetic acid. Observed mass 8213.62 Da; Calculated mass=8216.64 Da (average isotope composition).

b. Synthesis of Water-Soluble Polymer GP29

i. Synthesis of Template GRFNP17 Carrying Multiple Thiol Groups

A branching core template GRFNP 17 was synthesized manually on an amide generating (4-methyl)benzhydrylamine(MBHA)-resin on a 0.4 mmol scale. Boc-Lys(Fmoc)-OH was coupled using standard coupling protocols (Schnölzer, M., Int J Pept Protein Res. (1992) 40:180-93). 2.1 mmol amino acid, 10% DIEA in 3.8 ml 0.5M HBTU; i.e. 5-fold excess of amino acid. After removal of the Fmoc protecting group, Fmoc-Lys(Fmoc)-OH was coupled using standard amino acid coupling protocols (2.1 mmol amino acid, 10% DIEA in 3.8 ml 0.5M HBTU; i.e. 5-fold excess amino acid). After a second Fmoc removal step, Fmoc-Lys (Fmoc)-OH was coupled using standard amino acid coupling protocols (4.2 mmol amino acid, 10% DIEA in 7.6 ml 0.5M HBTU; i.e. 5-fold excess amino acid relative to free amine). After a final Fmoc deprotection step, a five-fold excess (relative to free amines) of S-acetyl thioglycolic acid pentafluorophenyl ester (SAMA-oPfp) in DMF was coupled for 30 minutes. The t-Boc protecting group of the C-terminal lysyl residue was removed by two times one minute batch washes with neat TFA, followed by neutralization of the resin by washing with 10% DIEA in DMF. 2 mmol Boc-aminooxyacetic acid and 2 mmol N-hydroxysuccinimide (NHS) were dissolved in 3 ml DMF. After addition of 2 mmol DIC (diisopropylcarbodiimide), the acid was activated for 30-60 minutes. The solution was added to the neutralized resin and coupled 1 hr. Finally, the S-linked acetyl groups were removed with 20% piperidine in DMF for 30 minutes. The template was deprotected and simultaneously cleaved from the resin support using HF/p-cresol according to standard Boc-chemistry procedures in the presence of cysteine as a scavenger for free aldehyde (Schnolzer, M., Int J Pept Protein Res. (1992) 40:180-93). The recovered polyamide in 50% B [i.e. 50% aqueous acetonitrile containing 0.1% TFA] (aldehyde free) was frozen and lyophilized. For purification, the template crude product was dissolved in 2 ml 50% B, and 100 ml 100% A [i.e. 0.1% TFA in water] was added to dilute the sample (Avoid guanidinium chloride or acetate addition, since the addition of aldehyde is guaranteed). The template was loaded onto a C4 preparative reverse-phase HPLC column equilibrated at T=40° C. at 3% B. Salts were eluted isocratically and the desired template, GRFNP 17 purified in a linear gradient. Fractions containing the desired product were identified by ES-MS, pooled and lyophilized.

ii. Synthesis of Branched Water-Soluble Polymer GRNP29

GRFNP29, a branched (TTD-Succ)₁₂ polymer of 15 kD molecular weight was synthesized by thioether-generating ligation of purified thiol-containing template GRFNP17 and a linear polymer GRFNP31, Br-acetyl-(TTD-Succ)₁₂₋carboxylate, where GRFNP31 was synthesized on a Sasrin carboxy-generating resin following standard protocols (Rose, K. et al., U.S. patent application Ser. No. 09/379,297; Rose, et al., J Am Chem Soc.(1999) 121: 7034), bromoacetylated and purified.

A 1.3× molar excess (over total thiols) of the purified GRFNP31, Br-acetylated (EDA-Succ)₁₂, and purified thiol-containing template GRFNP17 were jointly dissolved 0.1 M Tris —HCl/6 M guanidinium chloride, pH 8.7 at ˜10 mM concentration. After dissolution, the solution was diluted threefold (v/v) with 0.1 M Tris —HCl, pH 8.7 buffer. The ligation mixture was stirred at room temperature and the reaction monitored by reversed-phase HPLC and ES/MS. Additional GRFNP31 reactant was added on an as-needed basis until the desired reaction product was the major product. For workup, 3× (v/v to ligation mix) 0.1M acetate/6 M guanidinium chloride, pH 4 was added, and the solution was loaded onto a preparative C4 reverse-phase HPLC column, and purified with a linear gradient. Fractions containing pure GRFNP29 construct were identified using ES-MS, pooled and lyophilized.

C. Attachment of GP29 to the of Ligated, Full-Length Polypeptide

The lyophilized full-length polypeptide was dissolved and co-lyophilized with an equimolar amount of the aminooxyacetyl (AOA)-containing branched water-soluble polymer construct GP29 in 50% aqueous acetonitrile containing 0.1% TFA. The dried powder was dissolved and purified by reverse-phase HPLC to yield the full-length polypeptide with GP29 covalently attached by an oxime bond to the keto functionality on the modified side chain of the lysine at position 67. The polymer-modified peptide was separated from unmodified peptide and unreacted polymer by preparative gradient C4 reverse-phase HPLC. Fractions containing the desired polymer-modified product were identified by ES-MS and pooled. Observed mass=23,835.92 Da; Calculated mass=23,844.64 Da (average isotope composition). Alternatively, GP29 was covalently attached after folding the polypeptide, however this was observed to give a lower yield.

d. Folding and Purification of Polypeptide containing Oxime-Linked GP29

The full-length polypeptide with GP29 attached was folded with concomitant formation of 2 disulfide bonds in aqueous buffer containing a cysteine-cystine redox couple, and purified by reverse-phase HPLC as described above. The folded product was homogeneous on HPLC. Observed mass=23,832 Da; Calculated mass=23,840 Da (average isotope composition).

### Example 15

### Preparation of RANTES Analog G1806

The RANTES analog, G1806, depicted in FIG. 14, was synthesized as follows.

1. Preparation of n-nonanoyl-RANTES(2-33)(Tyr3Chg)-thioester Sequence:

(SEQ ID NO.: 39)

nonanoyl-PXSSDTTPCC FAYIARPLPR AHIKEYFYTS
GK-thioester

where X=L-Cyclohexylglycine (Chg)

Peptide segment n-nonanoyl-RANTES(2-33)(Tyr3Chg)-thioester (thioester also depicted as —COSR, where R is alkyl group) was synthesized on a thioester producing resin as described above, and then modified at the N-terminus by direct coupling of n-nonanoic acid to the resin. The peptide resin was cleaved with hydrogen fluoride and purified by reverse-phase HPLC to yield n-nonanoyl-RANTES(2-33) Tyr3Chg. Observed mass=4151.98 Da; Calculated mass=4152.6 Da (average isotope composition).

2. Preparation of RANTES(34-68)(Met67Lys(Lev))-Lys69(Palm)-Leu70 Sequence:

| | |
|---|---|
| CSNPAVVFVT RKNRQVCANP EKKWVREYIN | (SEQ ID NO.: 40) |
| SLEK(Lev)SK(Palm)L | |

where K(lev) is a levulinic acid-modified epsilon nitrogen of Lys67 of the Met67Lys change from wild type RANTES; and where K(Palm) is a palmitate-modified epsilon nitrogen of Lys69 attached through an amino-octanoic moiety, having the structure:
   [εNitrogen Lys69]-C(O)—CH₂—CH₂—CH₂—CH₂—CH₂—CH₂—CH₂—NH—C(O)—(CH₂)₁₄—CH₃

RANTES(34-68)(Met67Lys)-Lys69(Palm)-Leu70 was synthesized by conventional solid phase peptide synthesis on a Boc-Leu-OCH₂-Pam-resin using the in situ neutralization/HBTU activation protocols for Boc chemistry solid phase peptide synthesis as described above. After coupling Boc-Lys(Fmoc)-OH at position 69, the Fmoc group was removed with 20% piperidine in DMF. Fmoc-8-amino-octanoic acid was activated with HBTU/DIEA and coupled to the epsilon nitrogen of Lys69. After Fmoc group removal, palmitic acid was activated with 1-hydroxy-7-azabenzotriazole (HATU) and 1,3-di-isopropylcarbodi-imide and coupled to the resin. The chain assembly was then completed using standard Boc chemistry SPPS procedures as described above. After completion of chain assembly the Fmoc group on the epsilon nitrogen of Lys67 was removed with 20% piperidine in DMF treatment. Levulinic acid was activated as the symmetrical anhydride with 1,3-di-isopropylcarbodi-imide and coupled to the epsilon nitrogen of Lys67. The peptide resin was cleaved with hydrogen fluoride and the product purified by reverse-phase preparative HPLC to yield RANTES(34-68)(Met67Lys(Lev))-Lys69(Palmitate)-Leu70. Observed mass=4813.72 Da; Calculated mass=4813.81 Da (average isotope composition).

3. Preparation of G1806 Sequence:

(SEQ ID NO.: 41)

nonanoyl-PXSSDTTPCC FAYIARPLPR AHIKEYFYTS
GKCSNPAVVF VTRKNRQVCA NPEKKWVREY INSLEK(Lev-
GP29)SK(Palm)L

where X=L-Cyclobexylglycine (Chg), and where palmitate (“Palm”) fatty acid attachment is through an amino-octanoic moiety:
[εNitrogen Lys69]-C(O)—CH₂—CH₂—CH₂—CH₂—CH₂—CH₂—CH₂—NH—C(O)—(CH₂)₁₄—CH₃

a. Assembly of Fatty Acid Modified Full Length Polypeptide By Native Chemical Ligation

The N-terminal and C-terminal fully unprotected peptide segments were joined together by native chemical ligation as described above. The full-length polypeptide in reduced form was purified using reverse-phase HPLC with a linear gradient of acetonitrile versus water containing 0.1% trfluoroacetic acid. Observed mass=8598.21 Da; Calculated mass=8596.27 Da (average isotope composition).

b. Attachment of the Water-soluble Polymer GP29 to Ligated, Fatty Acid Modified Full Length Polypeptide

The lyophilized full length polypeptide was dissolved and co-lyophilized with an equimolar amount of the AOA-containing polymer GP29. The dried powder was dissolved and purified by reverse-phase HPLC to yield the full length polypeptide with GP29 covalently attached by an oxime bond to the keto functionality on the modified side chain of the lysine at position 67. Observed mass=24217 Da; Calculated mass=24,224. Da (average isotope composition). Alternatively, GP29 can be covalently attached after folding the polypeptide, however this was observed to give a lower yield.

C. Folding and Purification of Polypeptide containing Oxime-linked GP29 and Fatty Acid

The full length polypeptide with GP29 attached was folded with concomitant formation of 2 disulfide bonds in aqueous buffer containing a cysteine-cystine redox couple, and purified by reverse-phase HPLC as described above. The folded product was homogeneous on HPLC. Observed mass 24210.10 Da; Calculated mass=24,220.17 Da (average isotope composition).

FIG. 15 shows analytical data representative of the final folded, purified synthetic Rantes anaologs. In particular, a representative SD S-PAGE gel comparing the relative molecular weights of wild type (Wt) RANTES to synthetic chemokine analog RANTES G1806 under reducing (R) and non-reducing conditions (N) in shown in FIG. 15. The relative molecular weights are depicted on the left hand side of each gel, which corresponds to a molecular weight standard run on the same gel (not shown). Also shown is a representative RP-HPLC chromatogram of the folded, purified G1806 product. This illustrates the purity and increased relative molecular weight of the precision polymer-modified constructs compared to wild type, native RANTES.

### Example 16

### Cell Fusion Assays for G1755-01, G1755, G1805 and G1806

Cell fusion assays were performed to examine the anti-HIV activity of the various polymer-modified RANTES analogs. The procedures employed areas described in Example 9. Representative results are depicted in Table 3 below.

TABLE 3
HeLa P4-CCR5/HeLa-Env-ADA Cell Fusion Data

| Compound | EC50 |
|---|---|
| AOP-RANTES (2-68) | 1.81 × 10⁻⁹ M |
| NNY-RANTES (2-68) | 3.23 × 10⁻¹⁰ M |
| RANTES G1755-01 | 11.00 × 10⁻⁹ M |
| RANTES G1755 | 1.87 × 10⁻⁹ M |
| RANTES G1805 | 1.40 × 10⁻⁹ M |
| RANTES G1806 | 2.98 × 10⁻¹⁰ M |

These results demonstrate the substantial retention of anti-fusion activity following site-specific modification with either the linear or branched water-soluble polymer constructs. This result was surprising given the observed increase in activity of various RANTES analogs when a hydrophobic moiety, such as a fatty acid, was attached to the C-terminus, which supported the hypothesis that increasing the hydrophobic nature of the RANTES analogs in general would increase anti-fusion activity. In contrast, the water-soluble polymers used for modification, particularly the branched water-soluble polymers, were quite hydrophilic and negatively charged, yet only a marginal reduction in activity was observed that was well within the target potency range sought for in vivo activity. Even more unexpected was the relative retention in activity of the RANTES analogs modified with the large, negatively charged branched water-soluble polymer constructs compared to the smaller, linear water-soluble polymer constructs, and that the small linear and large branched polymer modified analogs had substantially the same effect on activity. This later observation is believed to be due in part to anti-aggregation properties when water-soluble polymer modifications are made internal to the pendant C-terminal residue of wild type RANTES at an aggregation region, as designed, and/or the localized anti-fusion properties of the water-soluble polymers.

The additional potency-increasing changes compensated for the marginal activity loss from attachment of water-soluble polymers, relative to AOP-RANTES and NNY-RANTES. These changes included the introduction of one or more hydrophobic amino acid derivatives to the N-terminus, and addition of a hydrophobic fatty acid moiety to the C-terminus. These results indicate that additional changes in combination with the monodisperse polyamide ethylene polymers (or other water-soluble polymers) can improve overall potency of NNY- and AOP-type modified chemokine molecules, such as AOP— or NNY-RANTES having the combined Pro2Thz and Tyr3Chg change to the N-terminus and/or the addition of a lipid moiety to the C-terminus.

### Example 17

### Pharmacokinetic Studies for G1755, G1805, and G1806

Pharmacokinetic studies of polymer-modified RANTES analogs were performed in male Sprague-Dawley rats as follows. RANTES analogs G1755, G1805, and G1806, prepared as described above (along with AOP-RANTES as control), were formulated for intravenous (IV) injection just prior to injection from lyophilized protein stock in phosphate buffered saline at pH 7.0. Formulations were prepared so as to provide equimolar doses of the RANTES analogs to individual test animals [400 μg/kg (AOP-RANTES); 510 μg/kg (G1755); 1210 μg/kg (G1805); and 1225 μg/kg (G1806) (μg analog/kg animal)]. Final concentrations were adjusted where necessary to provide a total dose volume of 1 ml/kg for each animal (i.e., dose volumes were held constant). Each formulated analog was administered intravenously via the tail of the rats on day 1 of the experiment, and each animal received a single dose.

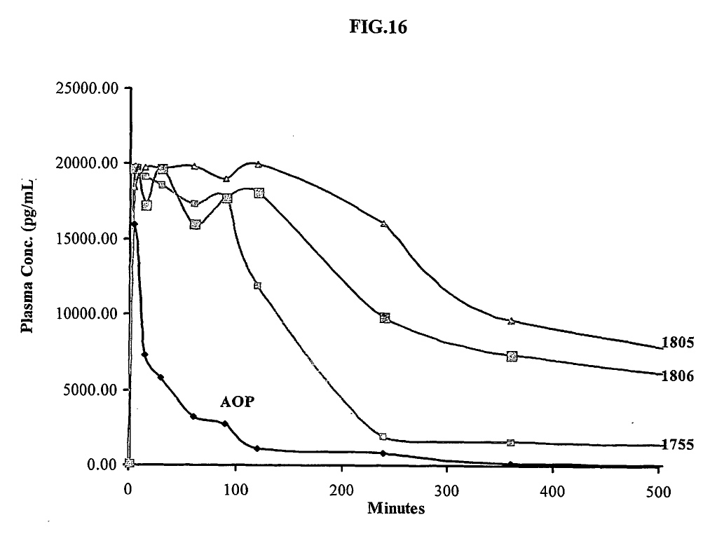

Following injection, approximately 0.25 ml of blood were collected from the tail vein/artery over the course of the experiment at each sample collection time point, and plasma or serum samples were prepared using EDTA as anticoagulant following standard protocols and the National Institutes of Health animal care guidelines and recommended testing procedures. Sample collection times were adjusted based on initial data collection to avoid collection of more than 14 samples of 0.25 ml from any animal within 3 weeks (as established by the National Institutes of Health animal care guidelines). Additional samples were collected at weekly intervals where detectable blood levels persisted beyond the initial samples. The plasma concentration of each RANTES analog at each time point was determined using Quantikine®Elisa, Human RANTES Immunoassay, (R&D Systems Catalog#DRNOO), per the manufacturers instructions. Overall health of the animals was monitored over the course of the experiment, including weight, eating habits, disposition, appearance etc., and no apparent side effects were observed. Representative results are depicted in FIG. 16.

These results demonstrate the substantive increase in circulating half-life provided by attachment of water-soluble polymers to the various precursor RANTES analogs, with the apparent increase in half-life of G1805>G1806>G1755>AOP-RANTES. Relative to AOP-RANTES, the increase was about 40-fold for G1805, 20-fold for G1806, and 10-fold for G1755. Collectively, the balance of retained high potency and significantly improved circulating half-life for the water-soluble polymer modified RANTES analogs is expected to enhance the therapeutic efficacy of these compounds in an in vivo setting, and reduce the number and amount of doses needed for treatment.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

1. A synthetic chemokine having a water-soluble polymer attached thereto and having in vitro bioactivity characterized by downmodulation of a chemokine receptor to which it binds.

2. The synthetic chemokine of claim 1, wherein said synthetic chemokine has an in vivo serum circulating half-life of greater than 10-fold compared to said synthetic chemokine that is devoid of said water-soluble polymer.

3. The synthetic chemokine of claim 1, wherein said synthetic chemokine is modified (A) at its N-terminus with a moiety selected from the group consisting of an aliphatic chain, an amino acid, and an amino acid derivative; or (B) at its C-terminus with a moiety selected from the group consisting of an aliphatic chain, a polycyclic, an amino acid, and an amino acid derivative; or (C) at both its N-terminus with a moiety selected from the group consisting of an aliphatic chain, an amino acid, and an amino acid derivative, and at its C-terminus with a moiety selected from the group consisting of an aliphatic chain, a polycyclic, an amino acid, and an amino acid derivative.

4. The synthetic chemokine of claim 3, wherein said synthetic chemokine is modified at its N-terminus with a moiety selected from the group consisting of an aliphatic chain, an amino acid, and an amino acid derivative.

5. The synthetic chemokine of claim 3, wherein said synthetic chemokine is modified at its C-terminus with a moiety selected from the group consisting of an aliphatic chain, a polycyclic, an amino acid, and an amino acid derivative.

6. The synthetic chemokine of claim 3, wherein said synthetic chemokine is modified at both its N-terminus with a moiety selected from the group consisting of an aliphatic chain, an amino acid, and an amino acid derivative, and at its C-terminus with a moiety selected from the group consisting of an aliphatic chain, a polycyclic, an amino acid, and an amino acid derivative.

7. The synthetic chemokine of any of claims 1 or 3, wherein said water-soluble polymer is attached to said synthetic chemokine at a site selected from the group consisting of a C-terminal site, aggregation site, glycosylation site, and glycosaminoglycan (“GAG”) binding site.

8. The synthetic chemokine of any of claims 1 or 3, wherein said synthetic chemokine is an analog of Rantes, MIP1α, MIP1β, SDF-1, IL-8 or MCP-1.

9. The synthetic chemokine of claim 8, wherein said synthetic chemokine is an analog of Rantes, and said water-soluble polymer is attached to a C-terminal site.

10. The synthetic chemokine of claim 8, wherein said synthetic chemokine is an analog of Rantes, and said water-soluble polymer is attached at an aggregation site.

11. The synthetic chemokine of claim 8, wherein said synthetic chemokine is an analog of Rantes, and said water-soluble polymer is attached at a glycosylation site.

12. The synthetic chemokine of claim 8, wherein said synthetic chemokine is an analog of Rantes, and said water-soluble polymer is attached at a GAG site.

13. The synthetic chemokine of claim 8, wherein said synthetic chemokine is an analog of MIP1α, and said water-soluble polymer is attached to a C-terminal site.

14. The synthetic chemokine of claim 8, wherein said synthetic chemokine is an analog of MIP1α, and said water-soluble polymer is attached at an aggregation site.

15. The synthetic chemokine of claim 8, wherein said synthetic chemokine is an analog of MIP1α, and said water-soluble polymer is attached at a glycosylation site.

16. The synthetic chemokine of claim 8, wherein said synthetic chemokine is an analog of MIP1α, and said water-soluble polymer is attached at a GAG site.

17. The synthetic chemokine of claim 8, wherein said synthetic chemokine is an analog of MIP1β, and said water-soluble polymer is attached to a C-terminal site.

18. The synthetic chemokine of claim 8, wherein said synthetic chemokine is an analog of MIP1β, and said water-soluble polymer is attached at an aggregation site.

19. The synthetic chemokine of claim 8, wherein said synthetic chemokine is an analog of MIP1β, and said water-soluble polymer is attached at a glycosylation site.

20. The synthetic chemokine of claim 8, wherein said synthetic chemokine is an analog of MIP1β, and said water-soluble polymer is attached at a GAG site.

21. The synthetic chemokine of claim 8, wherein said synthetic chemokine is an analog of SDF-1, and said water-soluble polymer is attached to a C-terminal site.

22. The synthetic chemokine of claim 8, wherein said synthetic chemokine is an analog of SDF-1, and said water-soluble polymer is attached at a GAG site.

23. The synthetic chemokine of claim 8, wherein said synthetic chemokine is an analog of IL-8, and said water-soluble polymer is attached to a C-terminal site.

24. The synthetic chemokine of claim 8, wherein said synthetic chemokine is an analog of IL-8, and said water-soluble polymer is attached at a GAG site.

25. The synthetic chemokine of claim 8, wherein said synthetic chemokine is an analog of MCP-1, and said water-soluble polymer is attached to a C-terminal site.

26. The synthetic chemokine of claim 8, wherein said synthetic chemokine is an analog of MCP-1, and said water-soluble polymer is attached at an aggregation site.

27. The synthetic chemokine of claim 8, wherein said synthetic chemokine is an analog of MCP-1, and said water-soluble polymer is attached at a glycosylation site.

28. The synthetic chemokine of claim 8, wherein said synthetic chemokine is an analog of MCP-1, and said water-soluble polymer is attached at a GAG site.

29. The synthetic chemokine of claim 1, wherein said water-soluble polymer has the formula: U-B-Polymer-J
where:
U comprises a residue of a functional group covalently joined to said synthetic chemokine;
B is a branching core having three or more arms and may be present or absent;
Polymer is a substantially non-antigenic water-soluble polymer; and
J is a residue of pendant group having a net charge under physiological conditions selected from the group consisting of negative, positive and neutral.

30. The synthetic chemokine of claim 29, wherein one or more of U, B, Polymer and J are separated by a spacer or linker; said spacer or linker having the formula: U-s1-B-s2-Polymer-s3-J
where s1, s2, and s3 are spacer or linker moieties that may be the same or different, and may be individually present or absent.

31. The synthetic chemokine of claim 29, wherein U is a residue of a bond selected from oxime, amide, amine, urethane, ether, thioether, ester, hydrazide, oxazolidine, and thaizolidine.

32. The synthetic chemokine of claim 29, wherein one arm of B is joined to U, and a second arm of B is joined to Polymer.

33. The synthetic chemokine of claim 29, wherein B comprises four or more arms.

34. The synthetic chemokine of claim 33, wherein two or more of said arms comprise a residue of bond selected from oxime, amide, amine, urethane, thioether, ester, hydrazide, oxazolidine, and thaizolidine.

35. The synthetic chemokine of claim 33, wherein B comprises a branching core moiety selected from amino, carboxyl, and mixed amino-carboxyl.

36. The synthetic chemokine of claim 29, wherein Polymer is selected from the group consisting of polyalkylene oxide and polyamide alkylene oxide.

37. The synthetic chemokine of claim 29, wherein Polymer is a polyamide of the formula —[C(O)—X—C(O)NH—Y—NH]n— or —[NH—Y—NH—C(O)—X—C(O)]n—, where X and Y are divalent radicals that may be the same or different and may be branched or linear, and n is an integer from 1 to 100.

38. The synthetic chemokine of claim 37, wherein either or both of X and Y comprise a repeat unit selected from:
—(O—CH₂—CH₂)ₙ— and —(CH₂—CH₂—O)ₙ—
where n is an integer from 1 to 100.

39. The synthetic chemokine of claim 29, wherein J is an ionizable group.

40. The synthetic chemokine of claim 39, wherein said ionizable group is selected from carboxyl, amine, and hydroxyl.

41. The synthetic chemokine of claim 39, wherein said water-soluble polymer has a net negative charge.

42. The synthetic chemokine of claim 39, wherein said water-soluble polymer has a net positive charge.

43. The synthetic chemokine of claim 39, wherein said water-soluble polymer has a net neutral charge.

44. A bioactive synthetic chemokine comprising a water-soluble polymer attached thereto, and having an in vitro EC50 that is equivalent to or less than that of a corresponding wild type chemokine for a biological response comprising inhibition of viral infection, said EC50 being measured in an in vitro cell-based assay characterized by binding of said bioactive synthetic chemokine to one or more of its corresponding chemokine receptors, where one or more of said receptors is a co-receptor for viral infection.

45. The bioactive synthetic chemokine of claim 44, wherein said EC50 is less than a concentration selected from the group consisting of 1000 nM, 700 nM, 500 nM, 400 nM, 300 nM, 200 nM, 100 nM, 10 nM and 1 nM.

46. A bioactive synthetic chemokine comprising a water-soluble polymer attached thereto and having:
(1) an EC50 that is equivalent to or less than that of a corresponding wild type chemokine, said EC50 being measured in an in vitro viral infection assay characterized by binding of said bioactive synthetic chemokine to one or more of its corresponding chemokine receptors, where one or more of said receptors is a viral coreceptor, and
(2) a serum circulating half-life of greater than 10-fold compared to said synthetic chemokine that is devoid of said water-soluble polymer.

47. The bioactive synthetic chemokine of claim 46, wherein said EC50 is less than a concentration selected from the group consisting of 1000 nM, 700 nM, 500 nM, 400 nM, 300 nM, 200 nM, 100 nM, 10 nM and 1 nM.


SEQUENCE LISTING (41 sequences)

SEQ ID NO: 1 - protein, 92 aa, Homo Sapiens
GSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 2 - protein, 68 aa, Homo Sapiens
SPYSSDTTPCCFAYIARPLPRAHIKEYFYTSGKCSNPAVVFVTRKNRQVCANPEKKWVREYINSLEMS

SEQ ID NO: 3 - protein, 74 aa, Homo Sapiens
GPASVPTTCCFNLANRKIPLQRLESYRRITSGKCPQKAVIFKTKLAKDICADPKKKWVQDSMKYLDQKSPTPKP

SEQ ID NO: 4 - protein, 73 aa, Homo Sapiens
KSMQVPFSRCCFSFAEQEIPLRAILCYRNTSSICSNEGLIFKLKRGKEACALDTVGWVQRHRKMLRHCPSKRK

SEQ ID NO: 5 - protein, 76 aa, Homo Sapiens
QPDAINAPVTCCYNFTNRKISVQRLASYRRITSSKCPKEAVIFKTIVAKEICADPKQKWVQDSMDHLDKQTQTPKT

SEQ ID NO: 6 - protein, 76 aa, Homo Sapiens
QPVGINTSTTCCYRFINKKIPKQRLESYRRTTSSHCPREAVIFKTKLDKEICADPTQKWVQDFMKHLDKKTQTPKL

SEQ ID NO: 7 - protein, 82 aa, Homo Sapiens
GPDAVSTPVTCCYNVVKQKIHVRKLKSYRRITSSQCPREAVIFRTILDKEICADPKEKWVKNSINHLDKTSQTFILEPSCLG

SEQ ID NO: 8 - protein, 70 aa, Homo Sapiens
ASLAADTPTACCFSYTSRQIPQNFIADYFETSSQCSKPGVIFLTKRSRQVCADPSEEWVQKYVSDLELSA

SEQ ID NO: 9 - protein, 69 aa, Homo Sapiens
APMGSDPPTACCFSYTARKLPRNFVVDYYETSSLCSQPAVVFQTKRSKQVCADPSESWVQEYVYDLELN

SEQ ID NO: 10 - protein, 70 aa, Homo Sapiens
ASNFDCCLGYTDRILHPKFIVGFTRQLANEGCDINAIIFHTKKKLSVCANPKQTWVKYIVRLLSKKVKNM

SEQ ID NO: 11 - protein, 77 aa, Homo Sapiens
GTNDAEDCCLSVTQKPIPGYIVRNFHYLLIKDGCRVPAVVFTTLRGRQLCAPPDQPWVERIIQRLQRTSAKMKRRSS

SEQ ID NO: 12 - protein, 74 aa, Homo Sapiens
GDTLGASWHRPDKCCLGYQKRPLPQVLLSSWYPTSQLCSKPGVIFLTKRGRQVCADKSKDWVKKLMQQLPVTAR

SEQ ID NO: 13 - protein, 99 aa, Homo Sapiens
RVTKDAETEFMMSKLPLENPVLLDRFHATSADCCISYTPRSIPCSLLESYFETNSECSKPGVIFLTKKGRRFCANPSDKQVQVCMRMLKLDTRIKTRKN

SEQ ID NO: 14 - protein, 97 aa, Homo Sapiens
QPKVPEWVNTPSTCCLKYYEKVLPRRLVVGYRKALNCHLPAIIFVTKRNREVCTNPNDDWVQEYIKDPNLPLLPTRNLSTVKIITAKNGQPQLLNSQ

SEQ ID NO: 15 - protein, 74 aa, Homo Sapiens
TKTESSSRGPYHPSECCFTYTTYKIPRQRIMDYYETNSQCSKPGIVFITKRGHSVCTNPSDKWVQDYIKDMKEN

SEQ ID NO: 16 - protein, 111 aa, Homo Sapiens
SDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 17 - protein, 69 aa, Homo Sapiens
GPYGANMEDSVCCRDYVRYRLPLRVVKHFYWTSDSCPRPGVVLLTFRDKEICADPRVPWVKMILNKLSQ

SEQ ID NO: 18 - protein, 71 aa, Homo Sapiens
ARGTNVGRECCLEYFKGAIPLRKLKTWYQTSEDCSRDAIVFVTVQGRAICSDPNNKRVKNAVKYLQSLERS

SEQ ID NO: 19 - protein, 127 aa, Homo Sapiens
QGVFEDCCLAYHYPIGWAVLRRAWTYRIQEVSGSCNLPAAIFYLPKRHRKVCGNPKSREVQRAMKLLDARNKVFAKLHHNMQTFQAGPHAVKKLSSGNSKLSSSKFSNPISSSKRNVSLLISANSGL

SEQ ID NO: 20 - protein, 71 aa, Homo Sapiens
KPVSLSYRCPCRFFESHVARANVKHLKILNTPACALQIVARLKNNNRQVCIDPKLKWIQEYLEKALNRFKM

SEQ ID NO: 21 - protein, 77 aa, Homo Sapiens
VPLSRTVRCTCISISNQPVNPRSLEKLEIIPASQFCPRVEIIATMKKKGEKRCLNPESKAIKNLLKAVSKEMSKRSP

SEQ ID NO: 22 - protein, 77 aa, Homo Sapiens
AVLPRSAKELRCQCIKTYSKPFHPKFIKELRVIESGPHCANTEIIVKLSDGRELCLDPKENWVQRVVEKFLKRAENS

SEQ ID NO: 23 - protein, 103 aa, Homo Sapiens
TPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 24 - protein, 77 aa, Homo Sapiens
GPVSAVLTELRCTCLRVTLRVNPKTIGKLQVFPAGPQCSKVEVVASLKNGKQVCLDPEAPFLKKVIQKILDSGNKKN

SEQ ID NO: 25 - protein, 73 aa, Homo Sapiens
ASVATELRCQCLQTLQGIHPKNIQSVNVKSPGPHCAQTEVIATLKNGRKACLNPASPIVKKIIEKMLNSDKSN

SEQ ID NO: 26 - protein, 73 aa, Homo Sapiens
APLATELRCQCLQTLQGIHLKNIQSVKVKSPGPHCAQTEVIATLKNGQKACLNPASPMVKKIIEKMLKNGKSN

SEQ ID NO: 27 - protein, 73 aa, Homo Sapiens
ASVVTELRCQCLQTLQGIHLKNIQSVNVRSPGPHCAQTEVIATLKNGKKACLNPASPMVQKIIEKILNKGSTN

SEQ ID NO: 28 - protein, 76 aa, Homo Sapiens
QHHGVTKCNITCSKMTSKIPVALLIHYQQNQASCGKRAIILETRQHRLFCADPKEQWVKDAMQHLDRQAAALTRNG

SEQ ID NO: 29 - protein, 9 aa, Homo Sapiens
FAYIARPLP

SEQ ID NO: 30 - protein, 32 aa, Homo Sapiens
PYSSDTTPCCFAYIARPLPRAHIKEYFYTSGK

SEQ ID NO: 31 - protein, 37 aa, Homo Sapiens
CSNPAVVFVTRKNRQVCANPEKKWVREYINSLEMSKL

SEQ ID NO: 32 - protein, 69 aa, Homo Sapiens
PYSSDTTPCCFAYIARPLPRAHIKEYFYTSGKCSNPAVVFVTRKNRQVCANPEKKWVREYINSLEMSKL

SEQ ID NO: 33 - protein, 32 aa, Homo Sapiens; identical to SEQ ID NO: 30

SEQ ID NO: 34 - protein, 37 aa, Homo Sapiens; identical to SEQ ID NO: 31

SEQ ID NO: 35 - protein, 69 aa, Homo Sapiens; identical to SEQ ID NO: 32

SEQ ID NO: 36 - protein, 32 aa, Homo Sapiens
  MISC_FEATURE (2)..(2): X is L-cyclohexylglycine
PXSSDTTPCCFAYIARPLPRAHIKEYFYTSGK

SEQ ID NO: 37 - protein, 37 aa, Homo Sapiens
CSNPAVVFVTRKNRQVCANPEKKWVREYINSLEKSKL

SEQ ID NO: 38 - protein, 69 aa, Homo Sapiens
  MISC_FEATURE (2)..(2): X is L-cyclohexylglycine
PXSSDTTPCCFAYIARPLPRAHIKEYFYTSGKCSNPAVVFVTRKNRQVCANPEKKWVREYINSLEKSKL

SEQ ID NO: 39 - protein, 32 aa, Homo Sapiens; identical to SEQ ID NO: 36
  MISC_FEATURE (2)..(2): X is L-cyclohexylglycine

SEQ ID NO: 40 - protein, 37 aa, Homo Sapiens; identical to SEQ ID NO: 37

SEQ ID NO: 41 - protein, 69 aa, Homo Sapiens; identical to SEQ ID NO: 38
  MISC_FEATURE (2)..(2): X is L-cyclohexylglycine